(19) United States
(12) Patent Application Publication
(10) Pub. No.: US 2025/0311956 A1
(43) Pub. Date: Oct. 9, 2025

(54) ARTIFICIAL INTELLIGENCE ENABLED DISEASE PROFILING

(71) Applicants: The General Hospital Corporation, Cambridge, MA (US); The Broad Institute, Inc., Cambridge, MA (US); Beth Israel Deaconess Medical Center, Inc., Boston, MA (US)

(72) Inventors: Patrick T. Ellinor, Brookline, MA (US); Steven Lubitz, Boston, MA (US); Jennifer Ho, Boston, MA (US); Samuel F. Friedman, Cambridge, MA (US); Shaan Khurshid, Boston, MA (US); Puneet Batra, Cambridge, MA (US)

(73) Assignees: The General Hospital Corporation, Boston, MA (US); The Broad Institute, Inc., Cambridge, MA (US); Beth Israel Deaconess Medical Center, Inc., Boston, MA (US)

(21) Appl. No.: 19/169,958

(22) Filed: Apr. 3, 2025

Related U.S. Application Data
(60) Provisional application No. 63/574,105, filed on Apr. 3, 2024.

(51) Int. Cl. A61B 5/346; G16H 50/30; G16H 50/50; G16H 50/70

(52) U.S. Cl. CPC A61B 5/346; G16H 50/30; G16H 50/50; G16H 50/70

(57) ABSTRACT

Artificial intelligence enabled disease profiling is described. An electrocardiogram analysis module is configured to derive disease vectors for a plurality of diseases using electrocardiogram training data from both disease-negative and disease-positive individuals. A standardized input is generated, via a data preprocessor of the electrocardiogram analysis module, from an electrocardiogram recorded from an individual. The standardized input is encoded, by a deep learning autoencoder of the electrocardiogram analysis module, into an embedding, the embedding being a lower-dimensional latent space representation of features extracted from the standardized input. At least one disease risk score for the individual is generated, by a statistical modeling algorithm of the electrocardiogram analysis module, for the plurality of diseases based on the embedding and the disease vectors.

### RELATED APPLICATIONS

This application claims priority to U.S. Provisional Patent Application Ser. No. 63/574,105, filed Apr. 3, 2024, entitled “Artificial Intelligence Enabled Disease Profiling,” the entire disclosure of which is hereby incorporated by reference herein in its entirety.

### STATEMENT REGARDING GOVERNMENT SUPPORT

This invention was made with government support under Grant Nos. HL139731 and HL157635 awarded by the National Institutes of Health. The government has certain rights in the invention.

### TECHNICAL FIELD

The subject matter disclosed herein is related to utilizing electrocardiogram data to generate disease risk scores. Particular examples relate to providing a system, a computer-implemented method, and a device to utilize data obtained from electrocardiograms of a subject to assess whether a subject is suffering from or is likely to suffer from a disease using machine learning models.

### BACKGROUND

Cardiovascular disease is the leading cause of mortality in the United States, accounting for nearly one million deaths in 2020. It is widely recognized that much of the morbidity and mortality attributable to cardiovascular disease may be prevented through optimization of risk factors, including the identification and management of chronic cardiometabolic conditions such as hypertension, diabetes, and chronic kidney disease. However, recognition of prevalent disease remains a significant challenge, with an estimated 11 million Americans having undiagnosed hypertension despite adequate access to health care.

To this end, the 12-lead electrocardiogram (ECG), a relatively inexpensive diagnostic test that can be obtained in seconds in most ambulatory care settings, is well-suited as a potential tool for disease screening. Although originally developed to detect arrhythmia, the diagnostic utility of the ECG expanded rapidly to include the identification of coronary artery disease and other cardiac structural abnormalities. It has become clear that non-cardiac diseases, from electrolyte derangements to central nervous system pathology, cause characteristic changes in the ECG waveform. Moreover, efforts leveraging artificial intelligence (AI) have recently revealed that the ECG contains diagnostic and prognostic information that extends beyond traditional clinical interpretation. Specifically, AI-enabled ECG analysis can discriminate cardiac structure (e.g., low ejection fraction), presence of disease (cardiac amyloidosis), and even incidence of future disease (e.g., atrial fibrillation, all-cause mortality).

### SUMMARY

Artificial intelligence enabled disease profiling is described. An electrocardiogram analysis module is configured to derive disease vectors for a plurality of diseases using electrocardiogram training data from both disease-negative and disease-positive individuals. A standardized input is generated, via a data preprocessor of the electrocardiogram analysis module, from an electrocardiogram recorded from an individual. The standardized input is encoded, by a deep learning autoencoder of the electrocardiogram analysis module, into an embedding, the embedding being a lower-dimensional latent space representation of features extracted from the standardized input. At least one disease risk score for the individual is generated, by a statistical modeling algorithm of the electrocardiogram analysis module, for the plurality of diseases based on the embedding and the disease vectors.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

An understanding of the features and advantages of the present invention will be obtained by reference to the following detailed description that sets forth illustrative implementations, in which the principles of the invention may be utilized, and the accompanying drawings of which:

FIG. 1 is an illustration of an environment in an example implementation that is operable to employ artificial intelligence enabled disease profiling as described herein.

FIG. 2 depicts an example training process that may be used in generating the deep learning autoencoder of the ECG analysis module of FIG. 1.

FIG. 3 illustrates an example implementation of the encoder network of the deep learning autoencoder of FIG. 1 in greater detail.

FIG. 4 illustrates an example implementation of the decoder network of the deep learning autoencoder of FIG. 1 in greater detail.

FIG. 5 illustrates a block diagram of a first implementation of a decoder convolutional block structure that may be used in the decoder network.

FIG. 6 illustrates a block diagram of a second implementation of a decoder convolutional block structure that may be used in the decoder network.

FIG. 7 depicts an example implementation of using the deep learning autoencoder to assess electrocardiogram data for disease risk prediction.

FIG. 8 depicts an example of disease-positive and disease-negative data points in a two-dimensional t-distributed stochastic neighbor embedding (t-SNE) space.

FIG. 9 depicts an example of a vector component score calculation showing the geometric relationship between an ECG encoding and a disease vector in the latent space.

FIG. 10 depicts an example procedure for training and validating an autoencoder for electrocardiogram analysis according to one or more implementations.

FIG. 11 depicts an example procedure for deriving and utilizing disease vectors from electrocardiogram data according to one or more implementations.

FIG. 12 depicts an example procedure for processing electrocardiogram data using a machine learning model according to one or more implementations.

FIG. 13 illustrates an example system including various components of an example device that can be implemented as any type of computing device as described and/or utilized with reference to FIGS. 1-12 to implement examples of the techniques described herein.

FIGS. 14A and 14B show an example of an autoencoder and phenotype vector derivation for latent space phenome-wide association studies (PheWAS).

FIGS. 15A and 15B show original ECG tracings and corresponding reconstructed median waveforms.

FIG. 16A shows a first uniform manifold approximation and projection (UMAP) stratified by age.

FIG. 16B shows a second UMAP stratified by sex and a third UMAP stratified by body mass index.

FIG. 17 illustrates an example comparing test statistic distributions and significant associations across different electrocardiogram analysis models.

FIG. 18 illustrates an analysis summarizing the top Phecode associations using 12-lead ECG and single-lead ECG models in a meta-analysis.

FIGS. 19A-19C illustrate an analysis summarizing the difference in the area under the receiver operator characteristic curve (AUC) between models with and without ECG vector component scores, stratified by Phecode category and dataset.

FIG. 20 illustrates an example of median waveform reconstructions for centroids reflecting individuals without and with left bundle branch block.

FIG. 21 illustrates an example of median waveform reconstructions for centroids reflecting individuals without and with hypokalemia.

FIG. 22 illustrates an example of median waveform reconstructions for centroids reflecting individuals without and with hypertrophic cardiomyopathy.

FIG. 23 illustrates an example of median waveform reconstructions for centroids reflecting individuals without and with rheumatoid arthritis.

The figures herein are for illustrative purposes only and are not necessarily drawn to scale.

### DETAILED DESCRIPTION

Unless defined otherwise, technical and scientific terms used herein have the same meaning as commonly understood by one of ordinary skill in the art to which this disclosure pertains. As used herein, the singular forms “a,” “an,” and “the” include both singular and plural referents unless the context dictates otherwise. The term “optional” or “optionally” means that the subsequently described event, circumstance, or substituent may or may not occur and that the description includes instances where the event or circumstance occurs and instances where it does not. The recitation of numerical ranges by endpoints includes all numbers and fractions subsumed within the respective ranges and the recited endpoints. The terms “about” or “approximately” as used herein when referring to a measurable value, such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the present disclosure. It is to be understood that the value to which the modifier “about” or “approximately” refers is also specifically disclosed.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and the progeny of a biological entity obtained in vivo or cultured in vitro are also encompassed.

Various implementations are described hereinafter. It should be noted that the specific implementations are not intended as an exhaustive description or as a limitation to the broader aspects discussed herein. One aspect described in conjunction with a particular implementation is not necessarily limited to that implementation and can be practiced with any other implementation(s). Reference throughout this specification to “one implementation,” “an implementation,” and “an example implementation” means that a particular feature, structure, or characteristic described in connection with the implementation is included in at least one implementation of the present invention. Thus, appearances of the phrases “in one implementation,” “in an implementation,” or “an example implementation” in various places throughout this specification are not necessarily all referring to the same implementation but may.

Furthermore, the particular features, structures, or characteristics may be combined in any suitable manner, as would be apparent to a person skilled in the art from this disclosure in one or more implementations. Furthermore, while some implementations described herein include some but not other features included in other implementations, combinations of features of different implementations are meant to be within the scope of the techniques described herein. For example, in the appended claims, any of the claimed implementations can be used in any combination.

### Overview

Current approaches to disease detection and risk assessment using electrocardiogram (ECG) data are limited in their ability to identify a wide range of medical conditions, particularly non-cardiac diseases. Traditional ECG analysis methods often rely on manual interpretation or simple statistical models, which may miss subtle patterns indicative of various health issues. These conventional techniques are typically focused on a narrow set of cardiac conditions and struggle to detect early signs of disease or assess risk across a broader spectrum of health problems.

Furthermore, existing ECG analysis methods often lack the ability to process and interpret large volumes of data efficiently, making it challenging to perform population-wide screening or personalized risk assessments. The limited scope and scalability of current solutions hinder their effectiveness in early disease detection, particularly for conditions where screening is currently cumbersome, inaccurate, or expensive.

To overcome these issues, artificial intelligence enabled disease profiling is disclosed herein. In accordance with the described techniques, an electrocardiogram analysis module is configured to derive disease vectors for a plurality of diseases using electrocardiogram training data from both disease-negative and disease-positive individuals and using a deep learning autoencoder. The electrocardiogram analysis module may generate a standardized input from an electrocardiogram recorded from an individual and encode the standardized input into an embedding using the deep learning autoencoder. The embedding is a lower-dimensional latent space representation of features extracted from the standardized input. The electrocardiogram analysis module may then generate at least one disease risk score for the individual for the plurality of diseases based on the embedding and the disease vectors.

In at least one implementation, the deep learning autoencoder comprises an encoder network and a decoder network. The encoder network and the decoder network may each include multiple convolutional blocks with skip connections. The encoder network may extract features from the standardized input electrocardiogram to generate the embedding in the latent space. The decoder network may reconstruct the original electrocardiogram from this embedding. Disease vectors may be derived by generating disease-positive and disease-negative embeddings from training data, plotting these in a two-dimensional space, determining centroids for the disease-positive and disease-negative embeddings, and defining vectors between the centroids. The individual's electrocardiogram embedding can then be projected onto these disease vectors to generate vector component scores, which are used to calculate disease risk scores.

This approach enables automated ECG analysis for identifying and monitoring disease risk across a wide range of medical conditions, which may be used in patient stratification, risk assessment, and treatment monitoring. The techniques described herein leverage ECG patterns associated with both cardiac and non-cardiac diseases that may not be apparent through traditional ECG analysis methods and/or manual interpretation (e.g., by a clinician). The techniques described herein allow for personalized and scalable disease detection, with the potential to generate likelihoods of disease at scale due to utilizing data from large and more diverse training samples. This may be particularly valuable for detecting diseases where screening is currently cumbersome, inaccurate, or expensive, and for which early detection could positively impact patient outcomes.

In one aspect, implementations disclosed herein provide methods to generate a disease risk score from a subject's waveform data using machine learning models. While there has been some recognition that noncardiac diseases can also manifest on the ECG, a need exists for a system, method and device that allows systematic evaluation of associations between waveform data, such as ECG-based features, and a broad range of disease phenotypes that uses a deep learning model to encode and reconstruct waveform data, including both 12-lead and single lead (lead I) ECG data. These methods and devices described herein provide for more reliable diagnosis of cardiac-related disease and other diseases based on an evaluation of waveform data, where current methods fail. More particularly, the methods, computer program products, systems, and devices described herein detect features in waveform data that could not be detected by a human merely by reading a waveform data printout, for example, an ECG printout. Further, the methods described herein provide a specific improvement over current methods, which only offer raw waveform data of a patient to assess a potential for cardiac disease, but which do not provide encoding and reconstructing waveform data obtained from a patient to detect and differentiate a wide variety of diseases, including cardiac and non-cardiac disease. The solutions provided herein are, therefore, non-conventional and technology-based.

The implementations described herein include computer-implemented methods, computer program products, and devices to use waveform data to generate one or more disease risk scores.

In some aspects, the techniques described herein relate to a system for electrocardiogram-based disease profiling, including: an electrocardiogram analysis module implemented in a non-transitory computer-readable storage medium, the electrocardiogram analysis module configured to perform operations including: deriving disease vectors for a plurality of diseases using electrocardiogram training data from both disease-negative individuals and disease-positive individuals for the plurality of diseases; generating, via a data preprocessor of the electrocardiogram analysis module, a standardized input from an electrocardiogram recorded from an individual; encoding, by a deep learning autoencoder of the electrocardiogram analysis module, the standardized input into an embedding, the embedding being a lower-dimensional latent space representation of features extracted from the standardized input; and generating, by a statistical modeling algorithm of the electrocardiogram analysis module, at least one disease risk score for the individual for the plurality of diseases based on the embedding and the disease vectors.

In some aspects, the techniques described herein relate to a system, wherein the operations further include: reconstructing, via a decoder network of the deep learning autoencoder, the electrocardiogram from the embedding.

In some aspects, the techniques described herein relate to a system, wherein the deep learning autoencoder includes an encoder network and a decoder network, the encoder network and the decoder network each including multiple convolutional blocks with skip connections.

In some aspects, the techniques described herein relate to a system, wherein deriving the disease vectors for the plurality of diseases includes, for each disease of the plurality of diseases: generating, via the deep learning autoencoder, disease-positive embeddings and disease-negative embeddings from the electrocardiogram training data; plotting the disease-positive embeddings and the disease-negative embeddings in a two-dimensional space; determining a disease-positive centroid and a disease-negative centroid in the two-dimensional space based on the plotted disease-positive embeddings and the plotted disease-negative embeddings, respectively; and defining a given disease vector by connecting the disease-negative centroid to the disease-positive centroid in the two-dimensional space.

In some aspects, the techniques described herein relate to a system, wherein generating the standardized input includes at least one of normalizing, upsampling, or zero-padding the electrocardiogram recorded from the individual.

In some aspects, the techniques described herein relate to a system, wherein generating the at least one disease risk score includes projecting the embedding onto the disease vectors in the lower-dimensional latent space.

In some aspects, the techniques described herein relate to a system, wherein the electrocardiogram recorded from the individual includes a 12-lead electrocardiogram.

In some aspects, the techniques described herein relate to a system, wherein the electrocardiogram recorded from the individual includes a single-lead electrocardiogram.

In some aspects, the techniques described herein relate to a system, wherein the at least one disease risk score indicates a likelihood of the individual having or developing at least one disease of the plurality of diseases within a specified time frame.

In some aspects, the techniques described herein relate to a system, wherein the operations further include: training the deep learning autoencoder using a training sample portion of a model derivation subset of the electrocardiogram training data; refining the trained deep learning autoencoder using a development sample portion of the model derivation subset of the electrocardiogram training data; and validating the deep learning autoencoder using both an internal sample portion of the model derivation subset of the electrocardiogram training data and an external test subset of the electrocardiogram training data.

In some aspects, the techniques described herein relate to a system, wherein training the deep learning autoencoder includes: generating, by the deep learning autoencoder, a reconstructed electrocardiogram based on an input electrocardiogram from the training sample portion; and updating weights and biases of the deep learning autoencoder based on a loss between the reconstructed electrocardiogram and the input electrocardiogram from the training sample portion.

In some aspects, the techniques described herein relate to a method for electrocardiogram-based disease profiling, including: deriving, using a trained deep learning autoencoder, at least one disease vector using electrocardiogram training data from both disease-negative individuals and disease-positive individuals for at least one disease; generating a standardized input for an electrocardiogram to be processed by the trained deep learning autoencoder; encoding, via an encoder network of the trained deep learning autoencoder, the standardized input into an embedding, the embedding being a lower-dimensional latent space representation of features extracted from the standardized input; generating at least one disease risk score based on the embedding and the at least one disease vector, the at least one disease risk score indicating a likelihood of the at least one disease being present; and reconstructing, via a decoder network of the trained deep learning autoencoder, the electrocardiogram from the embedding.

In some aspects, the techniques described herein relate to a method, wherein generating the at least one disease risk score includes: generating a vector component score by projecting the embedding onto a given disease vector of the at least one disease vector in the lower-dimensional latent space; and generating the at least one disease risk score for the given disease vector based on the vector component score.

In some aspects, the techniques described herein relate to a method, wherein deriving the at least one disease vector includes: generating disease-positive embeddings and disease-negative embeddings from the electrocardiogram training data; plotting the disease-positive embeddings and the disease-negative embeddings in a two-dimensional space; determining a disease-positive centroid and a disease-negative centroid in the two-dimensional space based on the plotted disease-positive embeddings and the plotted disease-negative embeddings; and defining the at least one disease vector as a vector connecting the disease-negative centroid to the disease-positive centroid in the two-dimensional space.

In some aspects, the techniques described herein relate to a method, wherein the trained deep learning autoencoder includes multiple convolutional blocks with skip connections in both of the encoder network and the decoder network.

In some aspects, the techniques described herein relate to a method, wherein generating the standardized input includes at least one of normalizing, upsampling, or zero-padding the electrocardiogram, and wherein the standardized input includes a time-series of voltage measurements for each lead of the electrocardiogram that are sampled at a predetermined frequency over a predetermined time period.

In some aspects, the techniques described herein relate to a method for electrocardiogram-based disease profiling, including: deriving disease vectors for a plurality of diseases using a trained deep learning autoencoder by, for a given disease of the plurality of diseases: generating, by the trained deep learning autoencoder, disease-positive embeddings from electrocardiograms corresponding to disease-positive individuals for the given disease; generating, by the trained deep learning autoencoder, disease-negative embeddings from electrocardiograms corresponding to disease-negative individuals for the given disease; plotting the disease-positive embeddings and the disease-negative embeddings in a two-dimensional space; determining a disease-positive centroid and a disease-negative centroid in the two-dimensional space based on the plotted disease-positive embeddings and the plotted disease-negative embeddings; and defining a given disease vector by connecting the disease-negative centroid to the disease-positive centroid in the two-dimensional space; and generating, using the trained deep learning autoencoder, disease risk scores for the plurality of diseases for an individual based on an electrocardiogram recorded from the individual and the disease vectors.

In some aspects, the techniques described herein relate to a method, wherein generating, using the trained deep learning autoencoder, the disease risk scores for the plurality of diseases for the individual based on the electrocardiogram recorded from the individual and the disease vectors includes: generating a standardized input from the electrocardiogram recorded from the individual by at least one of normalizing, upsampling, or zero-padding the electrocardiogram; encoding, via an encoder network of the trained deep learning autoencoder, the standardized input into an embedding; and for each disease of the plurality of diseases: generating a vector component score by projecting the embedding onto the given disease vector; and generating a corresponding disease risk score for the given disease based on the vector component score.

In some aspects, the techniques described herein relate to a method, wherein the trained deep learning autoencoder includes an encoder network and a decoder network, and the method further includes: reconstructing, via the decoder network of the trained deep learning autoencoder, the electrocardiogram from an embedding generated by the encoder network.

In some aspects, the techniques described herein relate to a method, wherein the trained deep learning autoencoder is trained using unsupervised learning.

### Example Environment

Turning now to the drawings, in which like numerals represent like (but not necessarily identical) elements throughout the figures, example implementations are described in detail.

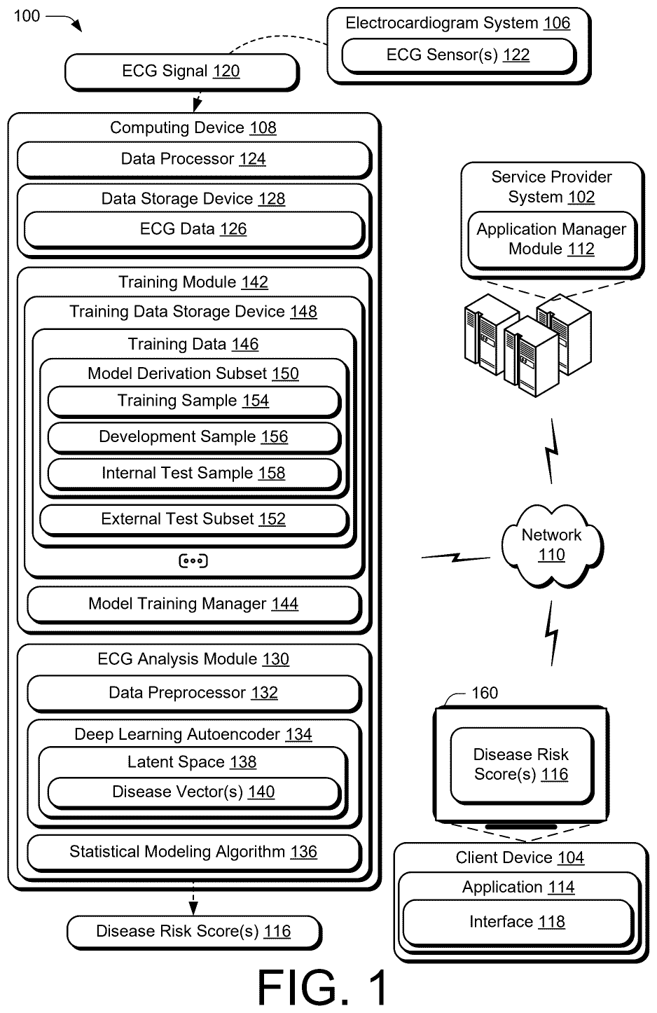

FIG. 1 is an illustration of an environment 100 in an example implementation that is operable to employ artificial intelligence enabled disease profiling as described herein. The illustrated environment 100 includes a service provider system 102, a client device 104, an electrocardiogram system 106, and a computing device 108 that are communicatively coupled, one to another, via a network 110. The network 110 may enable wired and/or wireless electronic communication, for example. Although the computing device 108 is illustrated as separate from the service provider system 102 and the client device 104, this functionality may be incorporated as part of the service provider system 102 and/or the client device 104, further divided among other entities, and so forth. By way of example, an entirety of or portions of the functionality of the computing device 108 may be incorporated as part of the service provider system 102 and/or the client device 104. Additionally, or alternatively, an entirety of or portions of the client device 104 may be incorporated as part of the service provider system 102 and/or the computing device 108. The client device 104 can interact with the web servers or other computing devices connected to the network 110, including the service provider system 102 and/or the computing device 108. In another example implementation, the client device 104 communicates with devices in the service provider system 102 and/or the computing device 108 via any other suitable technology.

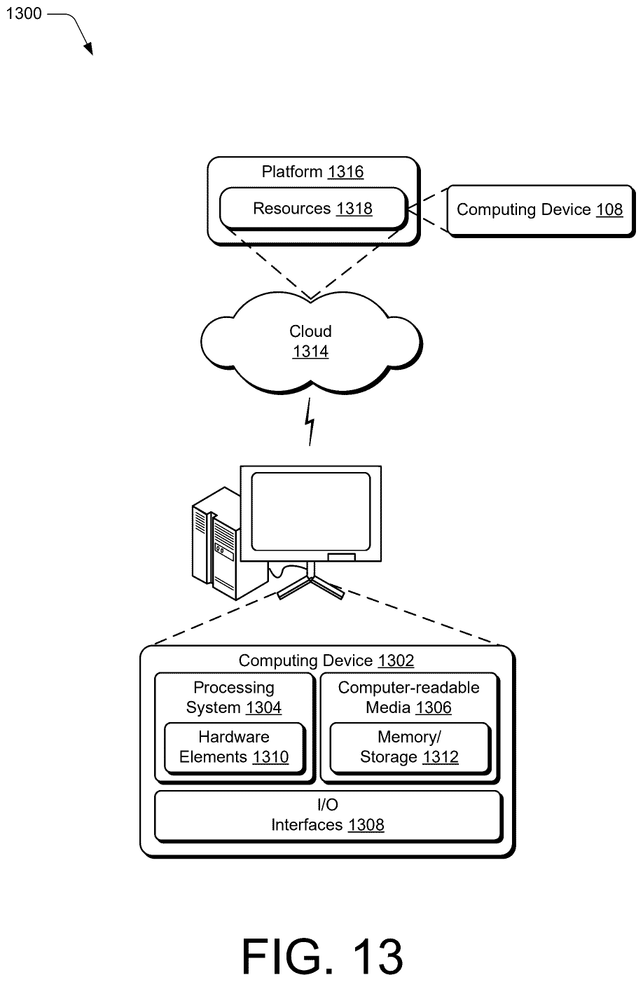

Computing devices that are usable to implement the service provider system 102, the client device 104, and the computing device 108 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers) to a low-resource device with limited memory and/or processing resources (e.g., mobile devices). Additionally, a computing device may be representative of a plurality of different devices, such as multiple servers utilized to perform operations “over the cloud,” as further described in relation to FIG. 13.

The service provider system 102 is illustrated as including an application manager module 112 that is representative of functionality to provide access to the computing device 108 to a user of the client device 104 via the network 110. The application manager module 112, for instance, may expose content or functionality of the computing device 108 that is accessible via the network 110 by an application 114 of the client device 104. The application 114 may be configured as a network-enabled application, a browser, a native application, and so on, that exchanges data with the service provider system 102 via the network 110. The data can be employed by the application 114 to enable the user of the client device 104 to communicate with the service provider system 102, such as to receive application updates and features when the service provider system 102 provides functionality to manage the application 114.

In the context of the described techniques, the application 114 includes functionality to train and/or use a machine learning model to analyze ECG data and output at least one disease risk score 116, as will be elaborated herein. By way of example, the at least one disease risk score 116 may include a probability score indicating the likelihood of an individual having or developing various diseases within a specified time frame. As used herein, the term “likelihood” may refer to a statistical measure or probability estimate of a particular disease being present. The probability score may be indicated as, for example, a numerical value (e.g., a value on a scale of 0 to 1) or a percentage (e.g., a percentage between 1 and 100%). Additionally, or alternatively, the at least one disease risk score 116 may include an indication of disease severity, categorizing the risk as low, moderate, or high. In some implementations, the at least one disease risk score 116 may also include recommendations for follow-up tests or interventions based on the predicted risk level. The user can use the application 114 on the client device 104 to view, download, upload, or otherwise access documents or web pages through the interface 118 via the network 110.

In the illustrated example, the application 114 includes an interface 118 that is implemented at least partially in hardware of the client device 104 for facilitating communication between the client device 104 and the computing device 108. By way of example, the interface 118 includes functionality to receive inputs to the computing device 108 from the client device 104 (e.g., from a user of the client device 104) and output information, data, and so forth from the computing device 108 to the client device 104, including the at least one disease risk score 116. Throughout the discussion of example implementations, it should be understood that the terms “data” and “information” are used interchangeably herein to refer to text, images, audio, video, or any other form of information that can exist in a computer-based environment. The interface 118 may display a graphical user interface and/or other information to a user to allow the user to interact with the application 114. The interface 118 may receive user input for data acquisition and/or machine learning and display results to the user. The interface 118 can display a webpage associated with the application 114 and/or the computing device 108. The interface 118 can provide input, configuration data, and other display directions by the application 114 and/or the computing device 108. In another example implementation, the interface 118 can be managed by the computing device 108, the service provider system 102, or others. In another example implementation, the interface 118 can be managed by the client device 104 and be prepared and displayed to the user based on the operations of the client device 104.

The computing device 108 illustrated in FIG. 1 is further configured to receive an ECG signal 120 from the electrocardiogram system 106. The electrocardiogram system 106 includes ECG sensors 122 configured to detect electrical activity of the heart of a subject (e.g., a patient) during an ECG recording. By way of example, the ECG sensors 122 may include one or more electrodes that detect voltage differences on the skin surface resulting from the heart's electrical activity. ECG signals are typically in the range of millivolts; the size of each electrical wave is termed the amplitude, and the number of cardiac cycles per minute is the heart rate. For medical applications, the frequency content of ECG signals typically lies within the range of 0.05-150 Hz. After the ECG sensors 122 detect the electrical signals from the body, these signals are amplified and filtered to produce the ECG signal 120. The ECG signal 120 may be in the form of a time-varying voltage signal, for instance.

The terms “record” or “recording” may be used herein to refer to acquiring data through the process of detecting and processing electrical signals from the heart. The term “data” may be used herein to refer to one or more datasets acquired with an electrocardiogram system, such as the electrocardiogram system 106. In at least one implementation, data acquired via the electrocardiogram system 106 is processed via a data processor 124 of the computing device 108 to generate ECG data 126, which may be stored in a data storage device 128. The ECG data 126 may comprise individual heartbeat waveforms as well as longer recordings, e.g., multi-lead ECG strips. The data storage device 128 may represent one or more databases and other types of storage capable of storing the ECG data 126. For example, data storage device 128 can include one or more tangible computer-readable storage devices. The data storage device 128 can be stored on the computing device 108 or logically coupled to the computing device 108. For example, the data storage device 128 can include on-board flash memory and/or one or more removable memory accounts or removable flash memory. In another example implementation, the data storage device 128 can reside in a cloud-based computing system. The data storage device 128 may also store a variety of other data, such as patient demographic information, electronic health record information, and so forth.

The electrocardiogram system 106 may communicate with the computing device 108 to transmit requested data, such as ECG waveform data. By way of example, the data processor 124 may process the ECG signal 120 in real-time during a recording session (e.g., a period of time where the ECG signal 120 is acquired via the electrocardiogram system 106), as the electrical signals are received and transmitted to the computing device 108. The term “real-time” is defined to include a procedure that is performed without intentional delay (e.g., substantially at the time of occurrence). In the context of electrocardiogram instance, real-time denotes generating the ECG data 126 substantially as the ECG signal 120 is acquired. As a non-limiting example, the electrocardiogram system 106 may acquire data at a real-time sampling rate ranging between 250 and 1000 Hz. However, it should be understood that the real-time sampling rate may be dependent on the specific application. Accordingly, when acquiring a relatively large amount of data, the real-time processing may be adjusted. Thus, some implementations may have real-time sampling rates that are considerably faster than 1000 Hz, while other implementations may have real-time sampling rates slower than 250 Hz. In at least one variation, the data may be stored temporarily in a buffer (not shown) during a recording session and processed in less than real-time by the data processor 124 in an offline operation.

The ECG data 126 generated by the computing device 108 from the ECG signal 120 may be updated at a same or similar rate at which the ECG signal 120 is acquired. The data storage device 128 may store the processed ECG data 126. In at least one implementation, the ECG data 126 are stored in a manner to facilitate retrieval thereof according to its order or time of acquisition. The data storage device 128 may comprise any known data storage medium. It is to be appreciated that while the data processor 124 and the data storage device 128 are illustrated as part of the computing device 108, in at least one variation, the data processor 124 and/or the data storage device 128 are part of the electrocardiogram system 106 and/or another computing device.

In one or more implementations, the data processor 124 may process the ECG signal 120 in different analysis modules (e.g., QRS detection, rhythm analysis, ST segment analysis, QT interval measurement, and the like) to extract various features and measurements. When multiple ECG leads are obtained, the data processor 124 may also be configured to analyze the relationships between different leads. For example, one or more modules may perform signal filtering, baseline wander removal, QRS complex detection, heart rate calculation, arrhythmia detection, ST segment analysis, T wave alternans analysis, and the like, and combinations thereof. The modules may include, for example, a feature extraction module to identify key points in the ECG waveform such as P waves, QRS complexes, and T waves. In ECG analysis, for instance, normal sinus rhythm may show a characteristic pattern of P waves, QRS complexes, and T waves, whereas various abnormalities may result in changes to this pattern. A display module may be provided that reads the ECG data 126 from the data storage device 128 and displays the ECG waveform or a derived measurement in real-time while a procedure (e.g., an ECG recording procedure) is being performed on the patient and/or after completion of the procedure.

Further, the components of the electrocardiogram system 106 and/or the computing device 108 may be coupled to one another to form a single structure, may be separate but located within a common room, or may be remotely located with respect to one another. For example, one or more of the modules described herein may operate in a data server that has a distinct and remote location with respect to other components of the electrocardiogram system 106 and/or the computing device 108, such as the ECG sensors 122. Optionally, the electrocardiogram system 106 may be a unitary system that is capable of being moved (e.g., portably) from room to room. For example, the electrocardiogram system 106 may include wheels, may be transported (e.g., on a cart), or may comprise a handheld device.

In at least one implementation, the ECG data 126, or a portion thereof, may be processed by an ECG analysis module 130. By way of example, the ECG analysis module 130 is representative of the functionality implemented at least partially in hardware of the computing device 108 to analyze the ECG data 126, such as one or more ECG recordings, and output the at least one disease risk score 116. In the example shown in FIG. 1, the ECG analysis module 130 includes a data preprocessor 132, a deep learning autoencoder 134, and a statistical modeling algorithm 136 for analyzing an output of the deep learning autoencoder 134 to generate the at least one disease risk score 116. The deep learning autoencoder 134 is a trained machine learning model. In at least one variation, the ECG analysis module 130 includes a different type of machine learning model in addition or as an alternative to the deep learning autoencoder 134. By way of example, the ECG analysis module 130 may include one or more deep learning models that correspond to different types of machine learning models, where the underlying models learn using the same or different approaches (e.g., supervised learning, unsupervised learning, and/or reinforcement learning). Alternatively, or in addition, the ECG analysis module 130 may include different deep learning models having a same model architecture but that are trained using different input data. By way of example, these models may include regression models (e.g., linear, polynomial, and/or logistic regression models), classifiers, neural networks, and reinforcement learning based models, to name just a few. In at least one implementation, the ECG analysis module 130 includes a first deep learning autoencoder trained to utilize multi-lead (e.g., 12-lead) ECG data and a second deep learning autoencoder trained to utilize single-lead ECG data.

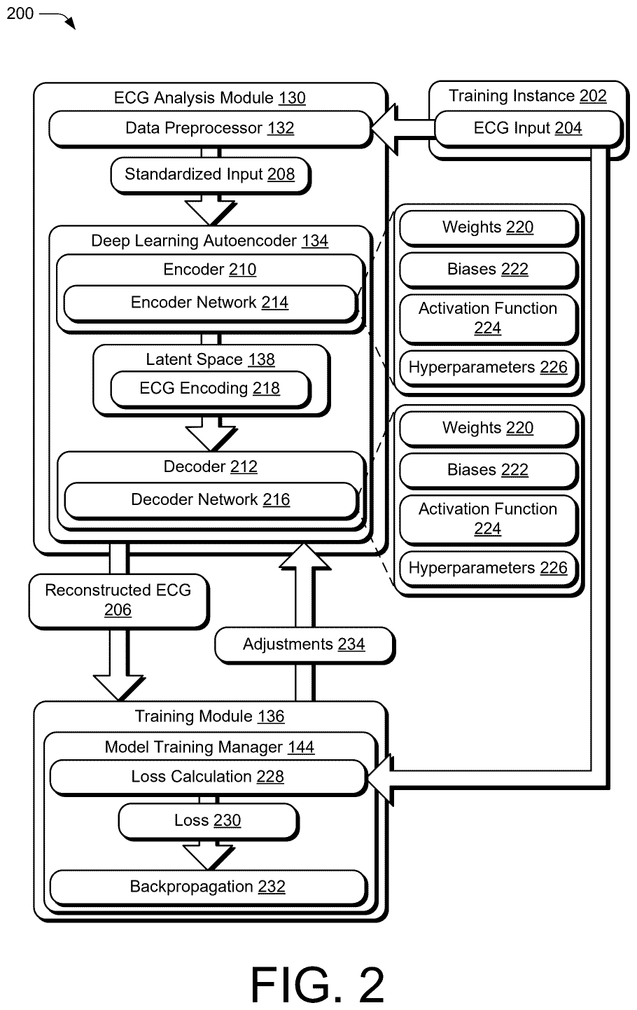

The deep learning autoencoder 134 may be configured as an unsupervised learning model that may encode the ECG data 126 into a lower-dimensional representation in latent space 138. The deep learning autoencoder 134 may also decode the lower-dimensional representation to reconstruct the original input. This approach may allow the model to “learn” important features and patterns in the ECG data 126 without labeled training data, e.g., by comparing the reconstructed output to the input. An example of this training process will be further described herein with respect to FIG. 2.

The deep learning autoencoder 134 may be configured as (or include) other types of models without departing from the spirit or scope of the described techniques. These different machine learning models may be built or trained (or the model otherwise learned), respectively, using different inputs and/or different algorithms due, at least in part, to different architectures and/or learning paradigms. Accordingly, it is to be appreciated that the following discussion of the functionality of the ECG analysis module 130 is applicable to a variety of machine learning models. For explanatory purposes, however, the functionality of the deep learning autoencoder 134 will be described generally with respect to convolutional neural networks (CNNs). The CNNs, for instance, may include one temporal dimension. By way of example, the deep learning autoencoder 134 may be based on a CNN architecture to process temporal information in ECG signals 120.

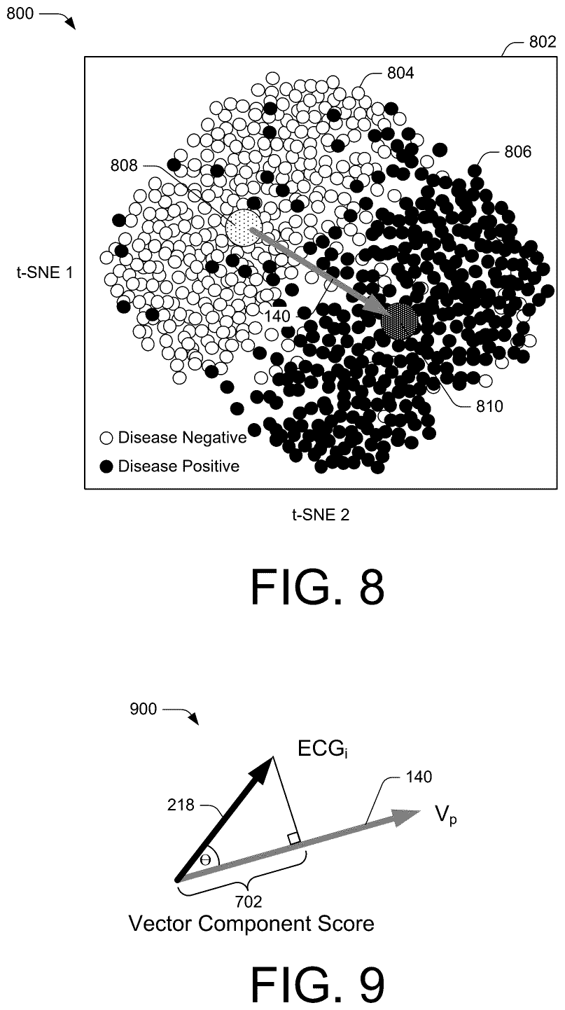

The encodings generated by the deep learning autoencoder 134 within the latent space 138 may be used by the ECG analysis module 130 to derive at least one disease vector 140, enabling associations with a range of medical conditions to be identified in the latent space 138. As will be further elaborated herein, e.g., with respect to FIGS. 8 and 9, the at least one disease vector 140 represents a specific vector in the latent space 138 that corresponds to a particular medical condition or disease state. The at least one disease vector 140 is derived from encodings generated by the deep learning autoencoder 134 corresponding to disease positive and disease negative states, for example.

In some implementations, the encodings generated by the deep learning autoencoder 134 in the latent space 138 may be used by the statistical modeling algorithm 136 to derive the at least one disease risk score 116. By way of example, by projecting individual ECG encodings onto the at least one disease vector 140, the ECG analysis module 130 may quantify the relationship between a given ECG and various disease states, enabling the calculation of the at least one disease risk score 116. That is, an ECG encoding generated from an individual's ECG may be projected onto a first disease vector that corresponds to a first disease, resulting in a first vector component score. The first vector component score may be further used by the statistical modeling algorithm 136 to generate a first disease risk score, the first disease risk score corresponding to a risk of the individual developing and/or having the first disease. Alternatively, or in addition, the ECG encoding may be projected onto a second disease vector that corresponds to a second disease, resulting in a second vector component score. The second vector component score may be further used by the statistical modeling algorithm 136 to generate a second disease risk, the second disease risk score corresponding to a risk of the individual developing and/or having the second disease.

The statistical modeling algorithm 136 may be implemented to process the lower-dimensional representations in the latent space 138. The statistical modeling algorithm 136 may employ various statistical techniques, such as logistic regression, Cox proportional hazards models, or other machine learning methods, to derive disease risk scores from the latent space encodings. The statistical modeling algorithm 136 may analyze the relationships between the latent space representations and known disease outcomes to build predictive models for various medical conditions based on the training data so that, subsequently, new ECG data 126 for which the disease outcome is unknown can be evaluated, as will be further described herein.

The computing device 108 further includes a training module 142 that is implemented at least partially in hardware of the computing device 108, at least in part, to deploy deep learning to generate the deep learning autoencoder 134. By way of example, the training module 142 includes a model training manager 144 that is configured to manage the deep learning autoencoder 134. This model management may include, for example, building the deep learning autoencoder 134, training the deep learning autoencoder 134, updating the model(s), and so forth. For instance, the model training manager 144 may be configured to carry out this model management using, at least in part, training data 146 maintained in a training data storage device 148. As illustrated in the environment 100 of FIG. 1, the training data 146 may include a model derivation subset 150 and an external test subset 152. For example, the model training manager 144 may use at least a portion of the model derivation subset 150 of the training data 146 as input for training the deep learning autoencoder 134 and may use at least a portion of the external test subset 152 for evaluating performance of the deep learning autoencoder 134 after the deep learning autoencoder 134 is at least initially trained. The model derivation subset 150 and the external test subset 152 may include ECG recordings from different data sources, for example. As such, the external test subset 152 may be used to verify that the deep learning autoencoder 134 achieves performance goals on data from diverse sources. Moreover, although the training data 146 may include multiple ECG recordings from a single patient, each separate recording may be treated as a separate training sample. Ellipses denote that more than one training data set may be stored in the training data storage device 148.

It is to be appreciated that although the model derivation subset 150 and the external test subset 152 are shown stored in the same training data storage device 148, in at least one variation, the model derivation subset 150 and the external test subset 152 are distributed among multiple storage locations. Alternatively, or in addition, the training data storage device 148 may be stored in a location that is external to the computing device 108 and accessed by the computing device 108 (e.g., over the network 110). As such, it is to be appreciated that the relative arrangement of the various modules and data storage devices in FIG. 1 is non-limiting, and variations are possible.

In one or more implementations, the model derivation subset 150 is further subdivided into a training sample 154, a development sample 156, and an internal test sample 158. By way of example, the training sample 154 may comprise a largest portion of the of the model derivation subset 150, while the development sample 156 and/or the internal test sample 158 may comprise a smallest portion of the model derivation subset 150. As a non-limiting example, the training sample 154 comprises 70% of the model derivation subset 150, the development sample 156 comprises 15% of the model derivation subset 150, and the internal test sample 158 comprises 15% of the model derivation subset 150, although other divisions are possible. The training sample 154, for instance, may comprise between 50% and 80% of the model derivation subset 150, the development sample 156 may comprise between 10% and 40% of the model derivation subset 150, and the internal test sample 158 may comprise between 5% and 30% of the model derivation subset 150.

Broadly speaking, the training sample 154 may be input to the deep learning autoencoder 134 during a training process, where the deep learning autoencoder 134 learns patterns and relationships in the data. During the training process, weights and parameters of the deep learning autoencoder 134 may be adjusted to reduce (e.g., minimize) errors between an output of the model and the original input ECG data. Following completion of the training process, the deep learning autoencoder 134 is able to accurately reconstruct the ECG data of the training sample 154.

The development sample 156 may be input to the deep learning autoencoder 134 during a model refinement (e.g., fine-tuning) process, where the deep learning autoencoder 134 is adjusted to prevent or reduce overfitting/underfitting of the model to the training sample 154. By way of example, the model refinement process may be performed following each round (or epoch) of training to evaluate how well the deep learning autoencoder 134 performs on data that is different from the training sample 154. During the model refinement process, for instance, a complexity, learning rate, and/or regularization of the deep learning autoencoder 134 may be adjusted (e.g., by the model training manager 144, automatically and/or based on user input) based on the performance of the deep learning autoencoder 134 with the development sample 156. As an illustrative example, if the deep learning autoencoder 134 accurately reconstructs the ECG data of the training sample 154 but not the development sample 156, overfitting of the deep learning autoencoder 134 to the training sample 154 is indicated. As such, the model refinement process enables settings of the deep learning autoencoder 134 and/or its training to be fine-tuned so that the deep learning autoencoder 134 can be generalized to unseen data (e.g., data that the deep learning autoencoder 134 has not been trained on).

The internal test sample 158 may be input to the deep learning autoencoder 134 during an internal validation process that is performed after the deep learning autoencoder 134 is trained and fine-tuned. The internal test sample 158 comprises data that was unseen by the deep learning autoencoder 134 during the training and model refinement processes described above but that is derived from the same dataset (e.g., the model derivation subset 150). The internal validation process evaluates the performance of the deep learning autoencoder 134 on similar data as to that used during the training and model refinement processes. If the deep learning autoencoder 134 does not meet acceptable or desired performance criteria (e.g., as defined by model developers) during the internal validation process, the deep learning autoencoder 134 may be returned to the training and/or model refinement processes so that changes can be made. For example, changes may be made to feature selection, the model architecture, regularization techniques, hyperparameter tuning, and the like.

The model training manager 144 may leverage the functionality of the data preprocessor 132 to process the training data 146 during the training, refinement, and validation processes described above. The data preprocessor 132, for instance, may remove patient identifying information and standardize ECG data input into the deep learning autoencoder 134 such as by normalizing, upsampling, and/or zero-padding the input electrocardiogram.

Once the deep learning autoencoder 134 is at least initially trained and internally validated, the model training manager 144 may use the external test subset 152 of the training data 146 to evaluate generalizability of the deep learning autoencoder 134. As mentioned above, the external test subset 152 comprises ECG data from a different data source, such as a different healthcare facility and/or patient population. By way of example, the external test subset 152 may be input into the deep learning autoencoder 134, and the deep learning autoencoder 134 may output latent space representations for respective ECG recordings. These latent space representations may be used to derive disease vectors and evaluate associations with various medical conditions. The external test subset 152, for instance, may be used to verify that the performance of deep learning autoencoder 134 is not specific to the data source of the model derivation subset 150.

Following training, the statistical modeling algorithm 136 may perform a meta-analysis by comparing the performance of the at least one disease risk score 116 across samples from the training data 146. The meta-analysis may evaluate the consistency and strength of associations between the at least one disease risk score 116 and various disease outcomes. This may include, for example, assessing the statistical power of the at least one disease vector 140 using metrics such as odds ratios, confidence intervals, and/or p-values. For instance, odds ratios for various conditions and/or diseases may be calculated and compared across datasets. The consistency of these associations across different cohorts may provide evidence for the robustness and generalizability of the corresponding at least one disease vector 140.

Additionally, the meta-analysis may include perturbation tests to validate the robustness of the associations. This may include randomly reclassifying phenotype labels (e.g., changing a disease-positive label to a disease-negative label, or vice versa) at different percentages and observing how the statistical significance of associations changes. This process may help demonstrate that the identified associations are not due to chance and are resistant to random noise in the data. The results of this meta-analysis may be used to identify which disease vectors demonstrate the strongest and most consistent predictive power across different populations and datasets. These disease vectors may be included as the at least one disease vector 140 in the deployed ECG analysis module 130.

For example, in response to the deep learning autoencoder 134 meeting desired or acceptable performance metrics, the deep learning autoencoder 134 may be deployed for analyzing newly obtained ECG data, including ECG data for which there are no associated clinical outcomes. By way of example, the ECG data 126 may correspond to ECG recordings that have not been evaluated by a clinician or technician with respect to specific disease associations. The ECG data 126 may be input into the (trained and validated) deep learning autoencoder 134 at or around the time of acquisition, and the deep learning autoencoder 134 may output latent space representations accordingly, thus enabling a streamlined ECG analysis workflow. These latent space representations may be used to generate the at least one disease risk score 116 based on the at least one disease vector 140 selected based on the meta-analysis.

In at least one implementation, the at least one disease risk score 116 may include probability scores indicating the likelihood of various diseases or risk of developing diseases within specified time frames. Additionally, or alternatively, the at least one disease risk score 116 may include estimated time frames for potential disease onset, which could range from months to years. In at least one implementation, the at least one disease risk score 116 additionally or alternatively includes an indication of disease severity, which may categorize the risk as low, moderate, or high. The at least one disease risk score 116 may further incorporate specific ECG features or patterns that contributed to the risk assessment, providing clinicians with actionable insights. In some implementations, the at least one disease risk score 116 may also include recommendations for follow-up tests or interventions based on the predicted risk levels.

Although the above discussion is focused on ECG inputs to the deep learning autoencoder 134 and receiving the at least one disease risk score 116 as the output, it is to be appreciated that additional inputs and/or outputs are possible. By way of example, the deep learning autoencoder 134 may output a reconstructed ECG for the ECG input.

The client device 104 is shown displaying, via a display device 160, at least one disease risk score 116. Alternatively, or in addition, the client device 104 may display, via the display device 160, the ECG data 126. It is to be appreciated that the at least one disease risk score 116 may be also stored in a memory of the computing device 108 and/or the client device 104 for subsequent access.

In this way, the ECG analysis module 130 enables automated ECG analysis for identifying and/or monitoring disease risk across a wide range of medical conditions, which may be used in patient stratification, risk assessment, and/or treatment monitoring. By way of example, the environment 100 generates at least one disease risk score 116 and reconstructs ECG waveform data of a subject using a machine learning model.

### Artificial Intelligence Enabled Disease Profiling

FIG. 2 depicts an example training process 200 that may be used in generating the deep learning autoencoder 134 of the ECG analysis module 130 of FIG. 1. It is to be appreciated that the example training process 200 denotes one implementation of a training process that may be used in generating at least a portion of the deep learning autoencoder 134.

In the example training process 200, a training instance 202 includes an ECG input 204, which is used by the deep learning autoencoder 134 to output a reconstructed ECG 206 of the ECG input 204. The ECG input 204, for instance, includes a sequence of voltage measurements obtained during a single ECG acquisition process. In at least one implementation, the ECG input 204 comprises a 12-lead ECG. In at least one variation, the ECG input 204 may comprise a single-lead ECG, a three-lead ECG, a five-lead ECG, a six-lead ECG, or another lead configuration. The specific lead configuration used may be selected based on factors such as the desired analysis, available equipment, or patient characteristics. During the training, the ECG input 204 is part of a corresponding portion of the training data 146 (e.g., the training sample 154).

Each ECG input 204 may be separately evaluated by the deep learning autoencoder 134 to generate the reconstructed ECG 206, which corresponds to the single ECG input 204. As such, the training instance 202 includes an input portion (e.g., the ECG input 204) and an associated expected output portion (e.g., the reconstructed ECG 206), and a great many training instances 202 may be used during the training process. The reconstructed ECG 206 corresponds to the attempt by the deep learning autoencoder 134 to reconstruct the ECG input 204, e.g., the original input ECG. The ECG input 204 thus serves as both the input and the true or expected output of the deep learning autoencoder 134. By way of example, the reconstructed ECG 206 may be compared to the original ECG input 204 to assess the reconstruction performance of the deep learning autoencoder 134 during training.

In the example shown in FIG. 2, the ECG input 204 is provided to the data preprocessor 132, which generates a standardized input 208. As mentioned above with respect to FIG. 1, the data preprocessor 132 may remove patient identifying information and standardize ECG data input into the deep learning autoencoder 134. For example, the data preprocessor 132 may apply filtering techniques to remove noise and baseline wander from the ECG signal. Alternatively or in addition, the data preprocessor 132 may normalize the amplitude of the ECG waveforms to a standard range. Additionally, the data preprocessor 132 may truncate or extend the ECG data to a predetermined time duration (e.g., 10 seconds), as warranted. By way of example, ECG data with shorter durations than the predetermined time duration may be zero-padded to include a standardized number of measurements. The data preprocessor 132 may upsample or downsample data to a predetermined frequency (e.g., 500 Hz). By way of example, data recorded at 250 Hz may be upsampled to 500 Hz. The data preprocessor 132 may also handle missing or corrupted data points through interpolation or other imputation methods, such as zero-padding. The preprocessing performed by the data preprocessor 132 may thus provide consistent, high-quality inputs to the deep learning autoencoder 134, which may improve the performance and generalizability of the deep learning autoencoder 134 across different ECG recording conditions and equipment.

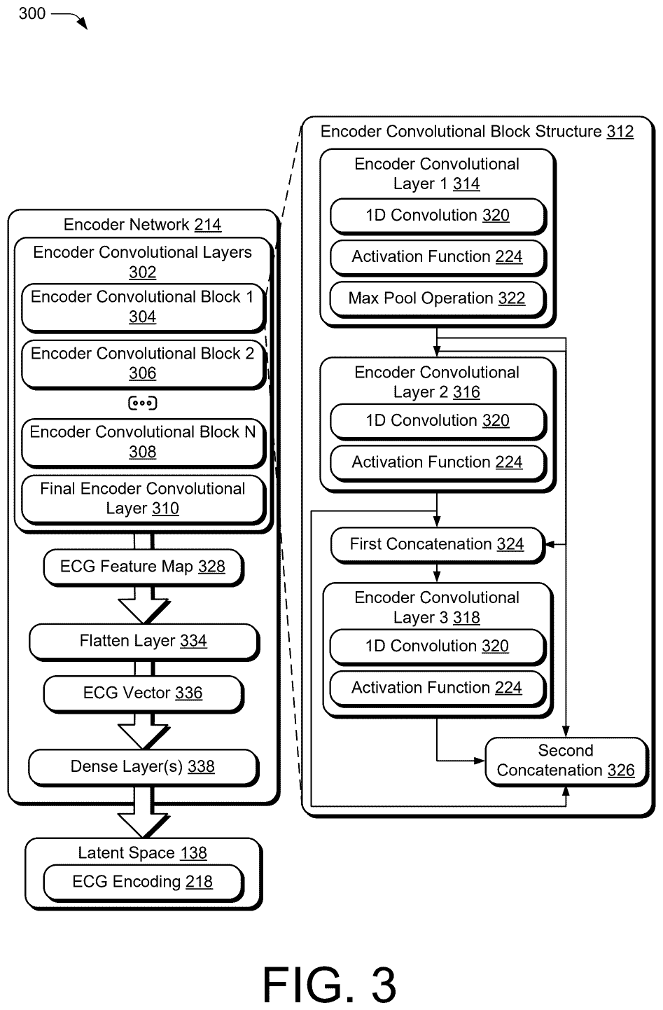

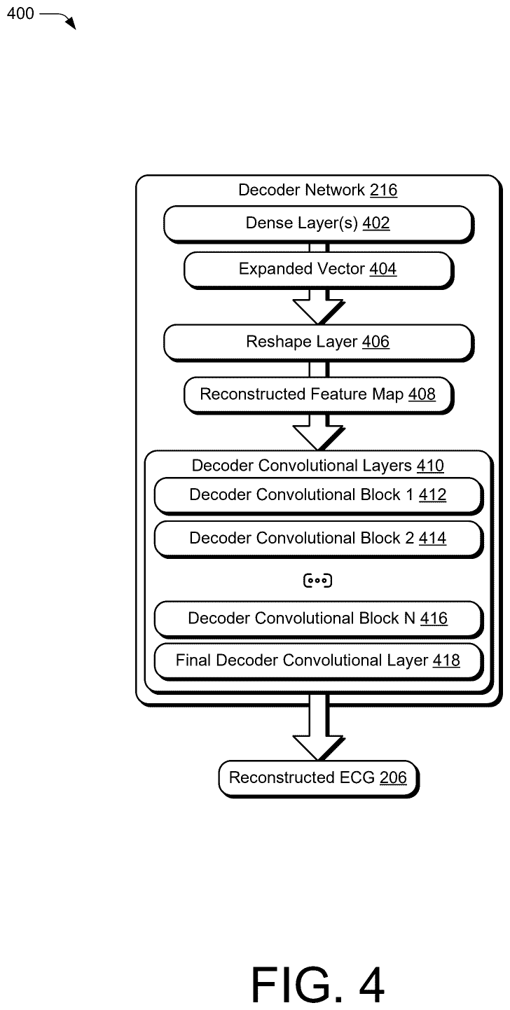

The standardized input 208 is provided to the deep learning autoencoder 134. As a non-limiting example, the standardized input 208 comprises a time-series of 5000 voltage measurements for each of the 12 leads, sampled at 500 Hz over 10 seconds. In general, the deep learning autoencoder 134 may include an encoder 210 and a decoder 212. In the implementation shown in FIG. 2, the encoder 210 includes an encoder network 214 configured to extract features of the standardized input 208, while the decoder 212 includes a decoder network 216 configured to reconstruct the original input based on the extracted features. An example architecture of the encoder network 214 is described below with respect to FIG. 3, and an example architecture of the decoder network 216 is described with respect to FIG. 4. The encoder network 214 and/or the decoder network 216 may be a convolutional neural network, for example.

As used herein in the context of machine learning, the term “feature” may refer to individual measurable properties, characteristics, and/or patterns of the input data that are used, evaluated, and/or “learned” by the deep learning autoencoder 134. Features may be numerical values extracted or computed from raw data, such as an electrocardiogram signal, that represent relevant aspects of the data for the task at hand. Initial features, for instance, may be values from the standardized input 208 itself, often represented as voltage readings over time. Each time step in the standardized input 208 is a potential feature that reflects the heart's electrical activity at that instant. Extracted features (e.g., extracted by the encoder network 214) may include, for example, basic shapes or waveforms in the ECG, like peaks or troughs; temporal patterns in the ECG that may reflect heartbeat rhythms or irregularities; and/or combinations of patterns that might correspond to higher-level physiological or health indicators. In the deep learning autoencoder 134, features serve as the basis for learning data patterns and relationships, allowing the model to generalize from the training data 146 to make predictions on new, unseen data.

The encoder network 214 and/or the decoder network 216 may each comprise weights 220, biases 222, an activation function 224, and hyperparameters 226. By way of example, the weights 220 and the biases 222 may be randomly initialized and then “learned” during the training process, as elaborated below. The encoder network 214, for instance, may perform a series of convolutions to compress the input data, while the decoder network 216 may use transposed convolutions (also known as deconvolutions) to reconstruct the original input. In each case, the convolutions may be performed in a manner that is dependent on the weights 220, the biases 222, the activation function 224, and the hyperparameters 226. By way of example, the weights 220, the biases 222, the activation function 224, and the hyperparameters 226 influence how features are extracted by the encoder network 214 and reconstructed via the decoder network 216.

For example, a convolution is a mathematical operation where a kernel slides over an input ECG signal and performs element-wise multiplication with the values of the signal at each temporal position. The results are summed up to produce a single output value for that temporal position, and this process is repeated across the ECG signal (e.g., the standardized input 208) to produce a feature map. The kernel comprises a matrix of numbers, which are the weights 220 of the kernel, that is applied to the input. The bias 222 is a single number added to the result of the convolution. After each convolution (or transposed convolution in the case of the decoder network 216), the feature map may be passed through the activation function 224, which may introduce non-linearity. In at least one implementation, the activation function 224 is a Mish activation function. After several convolution operations in the encoder network 214, a pooling layer may be used to reduce the size of the feature map. Conversely, in the decoder network 216, upsampling operations may be used to increase the spatial dimensions of the feature maps. The weights 220, biases 222, and/or activation function 224 may be the same or different for the encoder network 214 versus the decoder network 216 and/or for different convolutional layers of the respective networks.

The hyperparameters 226 are not learned during the training process but can be adjusted to increase performance. The hyperparameters 226 may comprise depth, stride, and zero-padding for both encoder network 214 and the decoder network 216. Depth controls the number of neurons within a given convolutional layer. Reducing the depth may increase the speed of the networks but may also reduce their accuracy. Stride determines how much the kernel moves or “slides” across the input. A stride of two means that the kernel moves two positions along the ECG signal (e.g., the standardized input 208) at each step, effectively skipping one sample each time. A higher stride value would thus enable the networks to process data more quickly and reduces the spatial dimensions of the feature map in the encoder (or increases them in the decoder). Zero-padding adds zeros around the edges of the input signal, allowing the kernel to be applied to edge samples where it would otherwise not fit, and ensuring that the output of the convolution remains the same size as the input when desired. The hyperparameters 226 may be the same or different for the encoder network 214 and the decoder network 216.

The encoder network 214 compresses the input data into a lower-dimensional representation in the latent space 138, indicated in FIG. 2 as an ECG encoding 218. The decoder network 216 attempts to reconstruct the ECG input 204 from this compressed representation. This process allows the deep learning autoencoder 134 to “learn” important features and patterns in the ECG data that enable accurate reconstruction without requiring labeled training data in a process referred to as unsupervised learning.

In at least one implementation, the encoder network 214 outputs the ECG encoding 218, which may be a one-dimensional vector that summarizes the features or characteristics of the standardized input 208 that have been “learned” by the encoder network 214. This ECG encoding 218 represents the input data in the latent space 138. The latent space 138 is a lower-dimensional representation of the input data, where similar ECGs are mapped to nearby points. The decoder network 216 receives the ECG encoding 218 and attempts to reconstruct the original input, generating the reconstructed ECG 206.

The reconstructed ECG 206 is compared to the ECG input 204 by the model training manager 144, which performs a loss calculation 228. The loss calculation 228 uses a loss function to compute the difference between the reconstructed ECG 206 and the ECG input 204, resulting in a loss 230. The loss 230 is a measure of the error of the deep learning autoencoder 134 in reconstructing the ECG input 204. Various types of loss functions may be used depending on the specific task and model architecture. By way of example, for reconstruction tasks, mean squared error or mean absolute error may be used.

A goal of the training is to minimize the loss 230 by adjusting the weights 220 and biases 222 of the deep learning autoencoder 134. To do this, the model training manager 144 employs backpropagation 232 to compute how the parameters are to be updated based on a gradient of the loss 230 with respect to each parameter. By way of example, the backpropagation 232 may utilize an optimization algorithm such as gradient descent (e.g., Adam). The backpropagation 232 results in adjustments 234, which are used to update the weights 220 and biases 222 of the deep learning autoencoder 134.

As such, following many rounds of training with a large number of training instances 202, the reconstructed ECG 206 becomes consistent with the ECG input 204 as the deep learning autoencoder 134 “learns” to minimize the loss 230 between the reconstructed ECG 206 and the ECG input 204.

In this way, the training process 200 provides a comprehensive framework for developing and refining the deep learning autoencoder 134 for ECG analysis and disease profiling. This approach may enable efficient learning from large datasets of ECG recordings, which may improve the accuracy and generalizability of disease risk predictions. The trained deep learning autoencoder 134 may be deployed in various clinical settings to analyze newly acquired ECG data from patients. For example, in a hospital or primary care clinic, the model could be integrated into existing ECG analysis workflows. When a patient undergoes an ECG recording, the data may be automatically processed by the ECG analysis module 130, providing immediate disease risk assessments for multiple conditions, including conditions for which the patient is not currently being treated and/or evaluated by a clinician. This real-time analysis may assist healthcare providers in several ways. As one example, patients at high risk of various diseases who may benefit from additional diagnostic tests or preventive interventions may be identified. For patients with known conditions, the disease risk predictions may aid in monitoring disease progression and assessing the effectiveness of current treatments.

FIG. 3 illustrates an example implementation 300 of the encoder network 214 of the deep learning autoencoder 134 of FIG. 1 in greater detail.

The encoder network 214 comprises encoder convolutional layers 302. In the present example shown in FIG. 3, the encoder convolutional layers 302 are grouped together in blocks. By way of example, a block may comprise multiple convolutional layers that are stacked together with operations such as pooling, batch normalization, and activation functions to form a unit that is able to extract more complex or abstract features than a single convolutional layer alone. The encoder convolutional layers 302 are shown as including a first encoder convolutional block 304 (e.g., “encoder convolutional block 1”), a second encoder convolutional block 306 (e.g., “encoder convolutional block 2”), and an N^{th} encoder convolutional block 308 (e.g., “encoder convolutional block N”), where N is an integer representing the total number of encoder convolutional blocks. Ellipses denote that additional encoder convolutional blocks may be present between the second encoder convolutional block 306 and the N^{th} encoder convolutional block 308. As a non-limiting example, there may be four encoder convolutional blocks. In at least one variation, there are fewer than three encoder convolutional blocks (e.g., two).

In at least one variation, one or more of the encoder convolutional layers 302 are arranged as single convolutional layers, such as a final encoder convolutional layer 310. For instance, the first encoder convolutional block 304, the second encoder convolutional block 306, and/or the N^{th} encoder convolutional block 308 instead may be a single convolutional layer. In at least one implementation, the encoder network 214 is a dense convolutional network, where each block connects to all subsequent blocks, allowing features from earlier blocks to be directly accessible to later layers.

The first encoder convolutional block 304, for example, may apply a kernel (e.g., a filter) to the standardized input 208, which scans across the dimension to extract low-level features like peaks, valleys, and edges across the ECG signal. The features detected by the first encoder convolutional block 304 may be fed to the subsequent encoder convolutional layers 302 (e.g., the blocks) in sequence, allowing the encoder network 214 to detect increasingly complex patterns in the standardized input 208. Accordingly, each block may progressively learn more complex features, helping the model understand the ECG data at multiple scales. By way of example, the second encoder convolutional block 306 may learn higher-level representations, like specific morphologies of waveforms, variations in wave amplitude, or frequency patterns, and the N^{th} encoder convolutional block 308 may learn global patterns that span across multiple heartbeats. Alternatively, or in addition, the encoder network 214 may perform progressive downsampling, where the number of input measurements decreases through striding or pooling operations. This may include reducing the temporal dimension to enable the deep learning autoencoder 134 to focus on capturing larger patterns and relationships in the data over longer time spans, while reducing the computational load.

FIG. 3 further illustrates an encoder convolutional block structure 312 that may be used in the encoder convolutional layers 302 of the encoder network 214. In the implementation 300, the encoder convolutional block structure 312 includes a first encoder convolutional layer 314 (e.g., “encoder convolutional layer 1”), a second encoder convolutional layer 316 (e.g., “encoder convolutional layer 2”), and a third encoder convolutional layer 318 (e.g., “encoder convolutional layer 3”). The first encoder convolutional layer 314 may include a 1D convolution 320, which applies a one-dimensional convolution operation to the input data. The output of the 1D convolution 320 may be passed through an activation function 224, which allows the encoder network 214 to model complex relationships in the data that a purely linear transformation may miss. The first encoder convolutional layer 314 may conclude with a max pool operation 322, which reduces the spatial dimensions of the feature maps and helps to achieve translation invariance. By way of example, the max pool operation 322 may divide the input into pooling regions and output the maximum value for each such region. This process may retain the most prominent features of the input data, thereby making the output of the first encoder convolutional layer 314 less sensitive to small translations or shifts in the input ECG signal. This property may enable the encoder network 214 to maintain consistent performance even when ECG features, such as QRS complexes or T waves, appear at different time points across various ECG samples.

The output from the first encoder convolutional layer 314 may be input into the second encoder convolutional layer 316. The second encoder convolutional layer 316 may also include the 1D convolution 320 followed by the activation function 224. The second encoder convolutional layer 316 may not include the max pool operation 322, in at least one implementation. This may allow the second encoder convolutional layer 316 to maintain the spatial dimensions of its input, which may potentially preserve more fine-grained features of the ECG signal. In some examples, omitting the max pool operation 322 in the second encoder convolutional layer 316 may enable the encoder network 214 to capture more complex, higher-level features. Additionally, or alternatively, this approach may help in retaining the full resolution of the features extracted by the first encoder convolutional layer 314, which may be beneficial for detecting subtle ECG abnormalities associated with various disease risks.

The outputs from the first encoder convolutional layer 314 and the second encoder convolutional layer 316 may be combined in a first concatenation 324. The first concatenation 324 may allow the network to preserve and utilize features from both the first encoder convolutional layer 314 and the second encoder convolutional layer 316, which may capture both low-level and higher-level features of the ECG signal.

The concatenated output may be processed by the third encoder convolutional layer 318, which follows a similar structure to the second encoder convolutional layer 316, with the 1D convolution 320 and the activation function 224. Similar to the second encoder convolutional layer 316, the third encoder convolutional layer 318 may not include the max pool operation 322.

The output from the third encoder convolutional layer 318 may be combined with the output from the first encoder convolutional layer 314 and the second encoder convolutional layer 316 in a second concatenation 326. The second concatenation 326 may allow the encoder convolutional block structure 312 to maintain a direct connection to the earliest features extracted from the input, which may help in preserving low-level information throughout the encoder convolutional block structure 312. In the example shown, the encoder convolutional block structure 312 incorporates skip connections in the first concatenation 324 and the second concatenation 326. These skip connections may enable information from earlier levels of the encoder convolutional block structure 312 to be preserved in the final output. This may provide a multi-scale representation of the input data and may help gradients flow more effectively during the backpropagation 232. Moreover, concatenating features from different layers in the encoder convolutional block structure 312 may enhance feature richness.

The specific configuration of the encoder convolutional block structure 312 may be adjusted based on the characteristics of the ECG data and the disease risk prediction task. For example, the number of filters in each convolutional layer, the size of the convolution kernels, the dropout rate, and the pooling size may be tuned to optimize performance. Additionally, multiple instances of this convolutional block structure 312 may be stacked to form deeper networks, which may allow for the extraction of more complex and abstract features from the ECG data.

The output of the encoder convolutional layers 302 may be an ECG feature map 328, which may be a multi-dimensional representation of the features extracted from the standardized input 208. The ECG feature map 328 may capture various characteristics of the ECG signal at different levels of abstraction, from low-level features like signal peaks and valleys to higher-level patterns such as waveform morphologies and rhythm irregularities. The ECG feature map 328 may be processed through a flatten layer 334, which may transform the ECG feature map 328 into an ECG vector 336. In some implementations, the dimensionality of the ECG vector 336 may be reduced compared to the ECG feature map 328, which may help in managing computational complexity while retaining information for disease risk prediction.

The ECG vector 336 is input into at least one dense layer 338, which are fully connected layers where every neuron (node) is fully connected to every neuron in the previous and next layer. The at least one dense layer 338 may function to aggregate information learned by previous layers and generate the ECG encoding 218 in the latent space 138. The at least one dense layer 338 may learn complex relationships between the features represented in the ECG vector 336, which may enable the model to capture subtle patterns that may be indicative of various disease risks. The at least one dense layer 338, for instance, may take the ECG vector 336 as an input vector, multiply it by a matrix of the weights 220, add the biases 222, and then apply the activation function 224 to produce the ECG encoding 218. It is to be appreciated that values of at least a portion of these parameters and/or type of activation function used are specific to a given encoder convolutional layer 302 or dense layer 338.

FIG. 4 illustrates an example implementation 400 of the decoder network 216 of the deep learning autoencoder 134 of FIG. 1 in greater detail.

The decoder network 216 includes a dense layer 402 that processes input data to produce an expanded vector 404. This dense layer 402 may take the low-dimensional ECG encoding 218 from the latent space 138 and expand it into a higher-dimensional vector. The expanded vector 404 connects to a reshape layer 406, which transforms the data into a reconstructed feature map 408. The reshape layer 406 may reorganize the expanded vector 404 into a multi-dimensional structure that can be processed by the subsequent convolutional layers, for instance. The reconstructed feature map 408 may provide a starting point for the reconstruction process of the ECG input 204.

The implementation 400 includes decoder convolutional layers 410 comprising a first decoder convolutional block 412, a second decoder convolutional block 414, and an N^{th} decoder convolutional block 416, where N is an integer representing the total number of decoder convolutional blocks. Ellipses denote that additional decoder convolutional blocks may be present between the second decoder convolutional block 414 and the N^{th} decoder convolutional block 416. As a non-limiting example, there may be four decoder convolutional blocks. In at least one variation, there are fewer than three decoder convolutional blocks (e.g., two). In at least one variation, one or more of the decoder convolutional layers 410 are arranged as single convolutional layers, such as a final decoder convolutional layer 418. For instance, the first decoder convolutional block 412, the second decoder convolutional block 414, and/or the N^{th} decoder convolutional block 416 instead may be a single convolutional layer.

The first decoder convolutional block 412, the second decoder convolutional block 414, and the N^{th} decoder convolutional block 416 may process data sequentially, with each block performing specific transformations on the input. The first decoder convolutional block 412, the second decoder convolutional block 414, and the N^{th} decoder convolutional block 416 may include operations such as transposed convolutions (also known as deconvolutions), upsampling, and/or activation functions. The structure of these blocks may approximately mirror the encoder convolutional blocks in reverse, with the goal of reconstructing the original ECG input 204 from the ECG encoding 218.

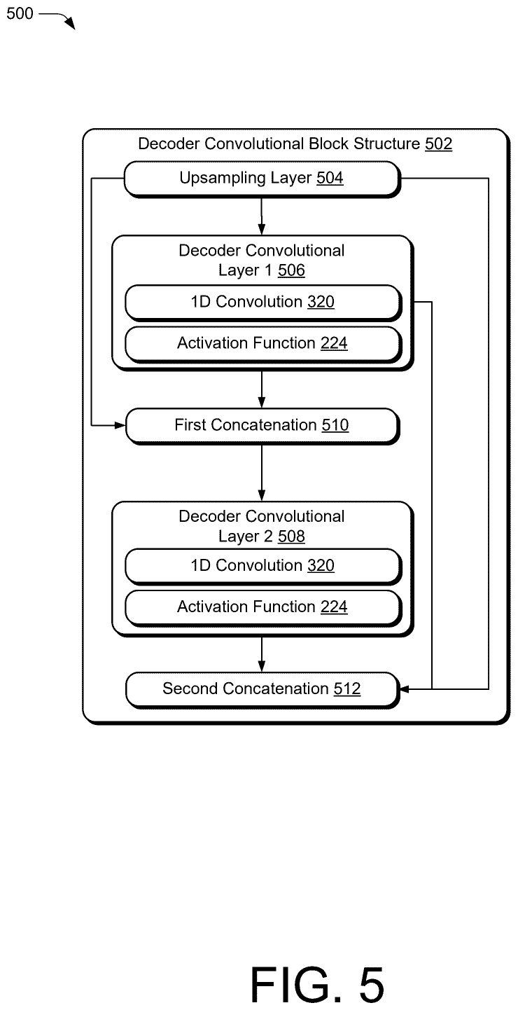

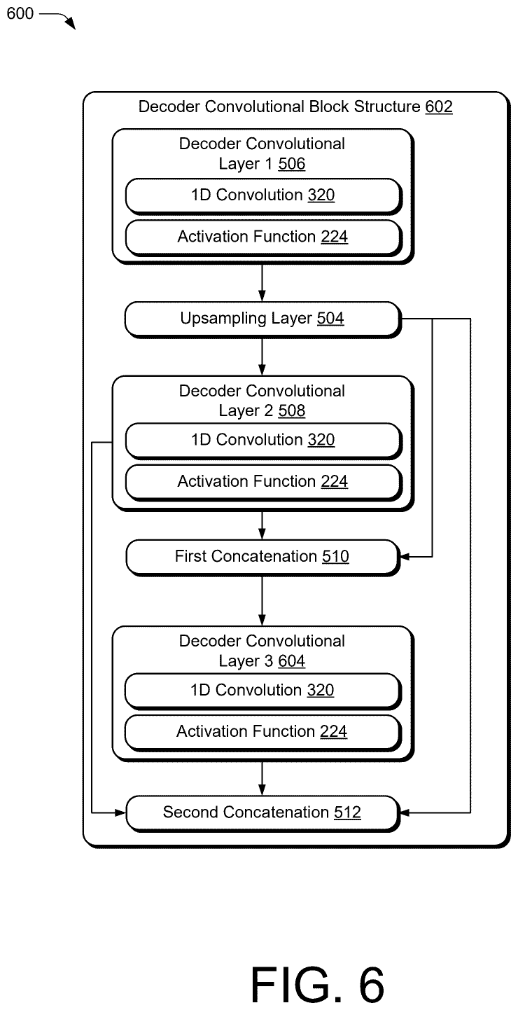

Similar to the encoder network 214, the decoder network 216 may incorporate skip connections and concatenation operations to preserve and utilize information from different stages of the reconstruction process, such as will be further illustrated with respect to the example convolutional block architectures of FIGS. 5 and 6.

The final decoder convolutional layer 418 produces the reconstructed ECG 206 as the output of the decoder network 216. This reconstructed ECG 206 aims to closely match the original ECG input 204, with the network learning to generate an accurate reconstruction through the training process 200.

FIG. 5 illustrates a block diagram of a first implementation 500 of a decoder convolutional block structure 502 that may be used in the decoder network 216. By way of example, the decoder convolutional block structure 502 may be used in the first decoder convolutional block 412, the second decoder convolutional block 414, and/or the N^{th} decoder convolutional block 416.

In the implementation 500, the decoder convolutional block structure 502 includes an upsampling layer 504, which increases the spatial dimensions of the input. The upsampling layer 504 is followed by a first decoder convolutional layer 506. Similar to the convolutional layers of the encoder network 214, the first decoder convolutional layer 506 includes the 1D convolution 320 and the activation function 224. The 1D convolution 320 applies learned filters to extract and combine features of the upsampled data. This operation may help refine the upsampled data, such as by recovering finer details lost during the encoding process performed by the encoder 210. The activation function 224 then introduces non-linearity, enabling the first decoder convolutional layer 506 to model complex relationships in the reconstructed data.

The output from the first decoder convolutional layer 506 may be input into a second decoder convolutional layer 508. The second decoder convolutional layer 508 may also include the 1D convolution 320 followed by the activation function 224. The second decoder convolutional layer 508 further processes the data, such as by refining the reconstructed features.

The outputs from the upsampling layer 504 and the first decoder convolutional layer 506 may be combined in a first concatenation 510. The first concatenation 510 merges the feature maps from these two layers, preserving both the upsampled data and the refined features, which may capture both low-level and higher-level features of the reconstructed ECG signal.

The concatenated output may be processed by the second decoder convolutional layer 508, which follows a similar structure to the first decoder convolutional layer 506, with the 1D convolution 320 and the activation function 224. The second decoder convolutional layer 508 operates on the combined feature map, enabling it to leverage both the original upsampled data and the refined features to produce a more accurate reconstruction.

The output from the second decoder convolutional layer 508 may be combined with the outputs from the upsampling layer 504 and the first decoder convolutional layer 506 in a second concatenation 512. The second concatenation 512 merges information from all previous layers within the decoder convolutional block structure 502. The second concatenation 512 may allow the decoder convolutional block structure 502 to preserve low-level information. This multi-scale approach to reconstruction may enable the decoder network 216 to produce more detailed and accurate outputs, for example.

FIG. 6 illustrates a block diagram of a second implementation 600 of a decoder convolutional block structure 602 that may be used in the decoder network 216. Similar to the first implementation 500 shown in FIG. 5, the decoder convolutional block structure 602 may be used in the first decoder convolutional block 412, the second decoder convolutional block 414, and/or the N^{th} decoder convolutional block 416.

Unlike the first implementation 500, where the decoder convolutional block structure 502 begins with the upsampling layer 504, the implementation 600 starts with the first decoder convolutional layer 506, which is followed by the upsampling layer 504. The upsampling layer 504 in both implementations may utilize methods such as nearest-neighbor interpolation or bilinear interpolation to expand the data.

Similar to the first implementation 500, the upsampled data passes through the second decoder convolutional layer 508. In the second implementation 600, the first concatenation 510 combines the outputs from the upsampling layer 504 and the second decoder convolutional layer 508. In contrast, in the first implementation 500 of FIG. 5, the first concatenation 510 merged the outputs from the upsampling layer 504 and the first decoder convolutional layer 506.

The implementation 600 includes a third decoder convolutional layer 604, which is not present in the first implementation 500. The third decoder convolutional layer 604, which also incorporates the 1D convolution 320 and the activation function 224, provides further processing of the concatenated feature maps.

In the second implementation 600, the second concatenation 512 combines outputs from the upsampling layer 504, the second decoder convolutional layer 508, and the third decoder convolutional layer 604. In contrast, in the first implementation 500 of FIG. 5, the outputs from the upsampling layer 504, the first decoder convolutional layer 506, and the second decoder convolutional layer 508 were combined in the second concatenation 512. Despite this differences, both implementations allow their respective decoder convolutional block structures to maintain connections to features from different processing stages, preserving multi-scale information throughout the reconstruction process.

Thus, the decoder convolutional block structure 602 differs from the decoder convolutional block structure 502 shown in FIG. 5 in its arrangement of layers and the placement of the upsampling layer 504. The specific configuration used for a given convolutional block may be chosen based on the characteristics of the ECG data and the desired reconstruction performance of the autoencoder. In at least one implementation, the first implementation 500 (FIG. 5) of the decoder convolutional block structure 502 may be employed in the initial stages of the decoder network 216. This configuration may be particularly suitable for the early expansion of the ECG encoding 218 from the latent space 138. By way of example, positioning the upsampling layer 504 at the beginning of the decoder convolutional block structure 502 facilitates the initial increase in spatial dimensions of the latent space representation.

In at least one implementation, the second implementation 600 (FIG. 6) of the decoder convolutional block structure 602 may be used in the later stages of the decoder network 216. The arrangement of the 1D convolution 320 preceding the upsampling layer 504 in the second implementation 600 allows for more intricate feature processing prior to increasing spatial dimensions, for instance. The decoder convolutional block structure 602 may be advantageous in reconstructing detailed aspects of the ECG waveforms, such as specific morphologies of P waves, QRS complexes, and T waves. As such, the decoder convolutional block structure 602 may be employed in layers closer to the output of the decoder network 216, where fine-grain details of the ECG signal are being reconstructed.

Both implementations incorporate skip connections through their respective concatenation operations (the first concatenation 510 and the second concatenation 512). These skip connections enable information to flow more directly across the decoder network 216, which may aid in preserving features from earlier stages of the decoding process. This approach may enhance the ability of the decoder network 216 to reconstruct complex ECG patterns throughout the decoding process.

Thus, the selection between these implementations at various stages and/or positions of the decoder network 216 may be based on the specific process of reconstructing ECG data at different levels of abstraction. For instance, earlier stages may focus on reconstructing broad waveform shapes, while later stages may prioritize fine temporal details and subtle variations in the ECG signal.

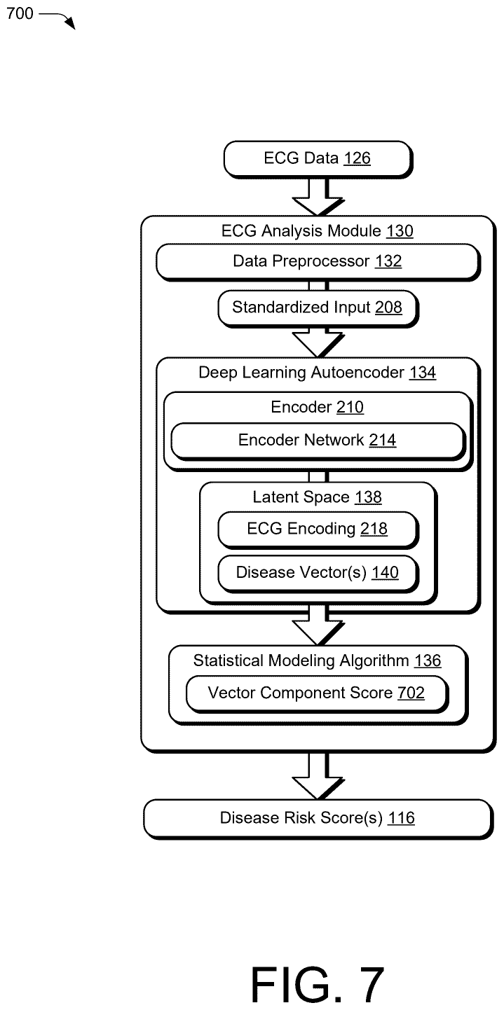

FIG. 7 depicts an example implementation 700 of using the deep learning autoencoder 134 to assess electrocardiogram data for disease risk prediction.

The implementation 700 includes processing newly acquired ECG data 126 from an individual via the ECG analysis module 130 to generate at least one disease risk score 116. The ECG data 126 are input into the ECG analysis module 130, where the data preprocessor 132 generates the standardized input 208. The standardized input 208 is processed by the deep learning autoencoder 134, which includes the encoder 210 having the encoder network 214. As described with respect to FIGS. 2 and 3, for instance, the encoder network 214 may include multiple blocks that progressively extract features from the ECG data 126, such as waveform morphologies, rhythm patterns, and temporal relationships.

The encoder network 214 outputs the ECG encoding 218 within the latent space 138, representing a lower-dimensional abstraction of the input ECG data 126. The ECG encoding 218 is then used to calculate a vector component score 702, e.g., by the statistical modeling algorithm 136. The vector component score 702 represents the projection of the ECG encoding 218 onto the at least one disease vector 140 in the latent space 138, quantifying the relationship between the individual's ECG and various disease states.

The statistical modeling algorithm 136 may further analyze the relationships of the vector component score 702 for a given disease and known disease outcomes to produce the at least one disease risk score 116. This approach allows the system to leverage the learned representations in the latent space 138 to generate risk predictions.

In the implementation 700, the at least one disease risk score 116 may include probability scores indicating the likelihood of having various diseases or risks of developing the various diseases within specified time frames. In at least one implementation, the at least one disease risk score 116 additionally or alternatively includes an indication of disease severity.

The at least one disease risk score 116 may be used to categorize patients into different risk levels, such as low, moderate, or high risk for specific diseases. This categorization may be based on comparing the calculated risk score for a given disease to one or more predetermined thresholds. For example, scores below a first threshold may indicate low risk, scores between the first threshold and a second threshold may indicate moderate risk, and scores above the second threshold may indicate high risk. These thresholds may be determined based on statistical analysis of population data, clinical guidelines, and/or other relevant factors. The specific thresholds and number of risk categories may vary depending on the particular disease being assessed and the clinical context in which the risk assessment is being used, for example.

In at least one implementation, population-level percentages may be used to stratify patients based on the at least one disease risk score 116. For example, low risk may refer to a first, lowest percentage range (e.g., bottom 30% of the population), intermediate risk may refer to a second percentage range (e.g., 30-75% of the population), and high risk may refer to a third, highest percentage range (e.g., top 25% of the population), although other divisions are possible. This approach may allow for a relative assessment of risk compared to the general population.

The at least one disease risk score 116 may be used for patient stratification, follow-up diagnostic assessment, and/or treatment monitoring across a wide range of medical conditions. For example, the implementation 700 may enable automated ECG analysis for identifying and/or monitoring disease risk for both cardiac and non-cardiac conditions. This approach may be particularly valuable for detecting diseases in which screening may be cumbersome, inaccurate, and/or expensive. Moreover, the ECG analysis module 130 may enable real-time assessments as the ECG data 126 is recorded, providing immediate disease risk assessments for multiple conditions, including conditions for which the patient is not currently being treated and/or evaluated by a clinician.

In at least one implementation, the decoder network 216 may produce the reconstructed ECG 206 so that it can be compared to the ECG data 126 for quality control purposes. For example, a poor reconstruction may indicate that the encoder network 214 has not accurately represented the ECG data 126 in the ECG encoding 218 and/or that the decoder network 216 has not accurately reconstructed the EGC from the ECG encoding 218.

Additional example details are described herein with respect to the Example Application.

FIG. 8 depicts an example 800 of disease-positive and disease-negative data points in a two-dimensional t-distributed stochastic neighbor embedding (t-SNE) space.

The example 800 includes a scatter plot 802 that displays a distribution of ECG encodings. The scatter plot 802 represents a two-dimensional projection of the latent space 138. By way of example, a t-SNE algorithm reduces the dimensionality of the latent space 138 while preserving local relationships between data points, allowing for visualization of complex high-dimensional data. The x-axis and y-axis of the scatter plot 802 represent the first and second t-SNE dimensions (e.g., t-SNE 1 and t-SNE 2), respectively. These dimensions do not correspond to specific ECG features but represent abstract dimensions that capture the variability and clustering in the latent space 138.

The scatter plot 802 shows disease-negative data points 804 represented by open (e.g., white-filled) circles and disease positive data points 806 represented by black-filled circles. A disease negative centroid 808 is positioned within the cluster of the disease-negative data points 804, while a disease positive centroid 810 is positioned within the cluster of the disease positive data points 806. The ECG encoding 218 data points are distributed across the t-SNE 1 and t-SNE 2 dimensions, forming distinct clusters based on disease status. The at least one disease vector 140 is represented by a line connecting the disease negative centroid 808 and the disease positive centroid 810, illustrating the direction in the latent space 138 that corresponds to the disease phenotype. The orientation and magnitude of the at least one disease vector 140 indicates how the disease manifests in the ECG latent space representation.

The clustering of the disease-positive data points 806 and the disease-negative data points 804 indicates that the deep learning autoencoder 134 has successfully identified distinguishing features in the ECG data that correlate with disease status. The at least one disease vector 140, connecting the disease negative centroid 808 and the disease positive centroid 810, represents the axis along which the model has learned to differentiate between healthy and diseased states most effectively.

FIG. 9 depicts an example 900 of a vector component score calculation showing the geometric relationship between an ECG encoding and a disease vector in the latent space 138. The example 900 includes the ECG encoding 218 projecting onto the at least one disease vector 140. The projection of the ECG encoding 218 onto the at least one disease vector 140 creates a vector component score 702. An angle θ is shown between the ECG encoding 218 and the at least one disease vector 140. The magnitude of the vector component score 702 is calculated using the angle θ, representing the position of the ECG encoding 218 along the at least one disease vector 140. This geometric representation in the example 900 illustrates how individual ECG measurements relate to disease phenotypes in the latent space 138 of the deep learning autoencoder 134, enabling quantitative assessment of disease risk based on the ECG encoding 218.

By way of example, the vector component score 702 may be calculated using the following formula:

$$VCS = \frac{{ECG}_{i} \cdot {V}_{p}}{‖ {V}_{p} ‖} = ‖ {ECG}_{i} ‖ cos ( θ )$$

where VCS is the vector component score 702, ECGᵢ is the ECG encoding 218, Vₚ is the at least one disease vector 140, and θ is the angle between them.

Having discussed example details of the techniques for artificial intelligence enabled disease profiling, consider now example procedures to illustrate additional aspects of the techniques.

### Example Procedures

This section describes example procedures for artificial intelligence enabled disease profiling in one or more implementations. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations, at least a portion of the procedure is performed by a suitably configured device, such as the computing device 108 of FIG. 1, by executing instructions stored in a non-transitory computer-readable storage medium.

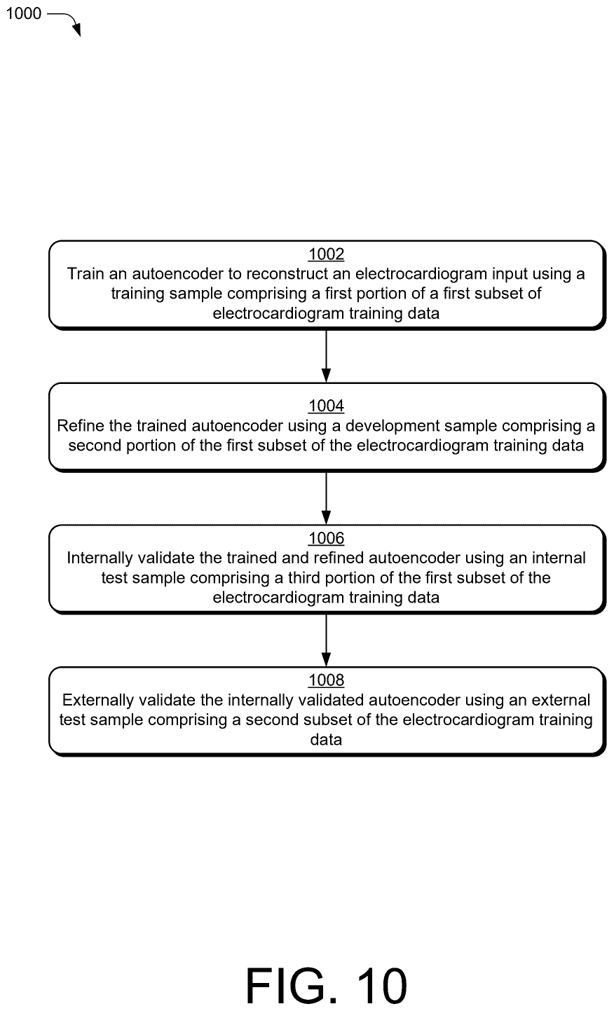

FIG. 10 depicts an example procedure 1000 for training and validating an autoencoder for electrocardiogram analysis according to one or more implementations. Where appropriate, reference will be made to components previously introduced in FIGS. 1-9.

An autoencoder is trained to reconstruct an electrocardiogram input using a training sample comprising a first portion of a first subset of electrocardiogram training data (block 1002). By way of example, the autoencoder may be included in the deep learning autoencoder 134 introduced with respect to FIG. 1. In one or more implementations, autoencoder includes encoder convolutional layers 302 for extracting features from standardized ECG inputs and decoder convolutional layers 410 for reconstructing the ECG data. The training module 142 may train the autoencoder using the training sample 154 of the model derivation subset 150 of the training data 146.

In at least one implementation, the training process includes exposing the autoencoder to a diverse set of ECG data from the model derivation subset 150. This allows the autoencoder to “learn” patterns in the ECG data associated with both encoding and decoding tasks. The encoder network 214 learns to compress the input data into the latent space 138, while the decoder network 216 learns to reconstruct the original input from this compressed representation.

The autoencoder “learns” via the adjustment of the weights 220 and biases 222 in both the encoder convolutional layers 302 and decoder convolutional layers 410. These adjustments are made through backpropagation 232 based on the calculated loss 230 between the reconstructed ECG 206 (output by the decoder network 216) and the ECG input 204 of the training instance 202. This iterative process continues until both the encoder and decoder achieve satisfactory performance on the model derivation subset 150 of the training data 146.

The trained autoencoder is refined using a development sample comprising a second portion of the first subset of the electrocardiogram training data (block 1004). By way of example, the model training manager 144 may use the development sample 156 to fine-tune the model hyperparameters 226 and adjust the weights 220 and biases 222 of both the encoder network 214 and decoder network 216. During refinement, the autoencoder is exposed to new ECG data from the model derivation subset 150, which is not seen by the autoencoder during the initial training process (e.g., as performed at block 1002). This process helps identify and correct any overfitting that may have occurred during the training process. The hyperparameters 226, such as the learning rate, batch size, and/or regularization strength, may be adjusted to achieve a desirable performance on the development sample 156 while maintaining good generalization to unseen data, for instance.

The trained and refined autoencoder is internally validated using an internal test sample comprising a third portion of the first subset of the electrocardiogram training data (block 1006). By way of example, the performance of the autoencoder may be evaluated on the internal test sample 158 to assess generalization. The internal validation process may provide an initial measure of an ability of the autoencoder to accurately reconstruct ECG data from a same data source as the training sample 154 and the development sample 156 (e.g., the model derivation subset 150) but that was not used during the training (block 1002) or refinement (block 1004) processes described above. During the internal validation process, for instance, the reconstructed ECG 206 may be compared against a corresponding ECG input 204 for the internal test sample 158. Various performance metrics may be calculated. The internal validation process may help identify any potential issues with the performance of the autoencoder and guide further refinement if the performance is not adequate.

The internally validated autoencoder is externally validated using an external test sample comprising a second subset of the electrocardiogram training data (block 1008). By way of example, in response to the autoencoder meeting an acceptable or desired performance criteria, an external validation process may be performed using the external test subset 152. During the external validation process, the external test subset 152 may be input to the internally validated autoencoder. The external test subset 152 may be used to verify that the performance of the deep learning autoencoder 134 is not specific to the data source of the model derivation subset 150. The external test subset 152, for instance, may comprise ECG data from one or more separate cohorts, often collected from a different institution and/or different patient population than the model derivation subset 150. The external validation process may provide an evaluation of the autoencoder with respect to a real-world scenario and may help identify biases or limitations that were not apparent in the internal validation. The external validation may provide confidence in the ability of the autoencoder to accurately reconstruct ECG data across diverse patient populations and clinical settings.

In this way, the procedure 1000 enables generation of an autoencoder that is able to reconstruct ECG inputs accurately. By leveraging the ECG waveform data, both the encoder network 214 and decoder network 216 “learn” to encode and reproduce latent information from ECGs that may not be readily interpretable by human observers with respect to a plurality of different diseases, as further described herein.

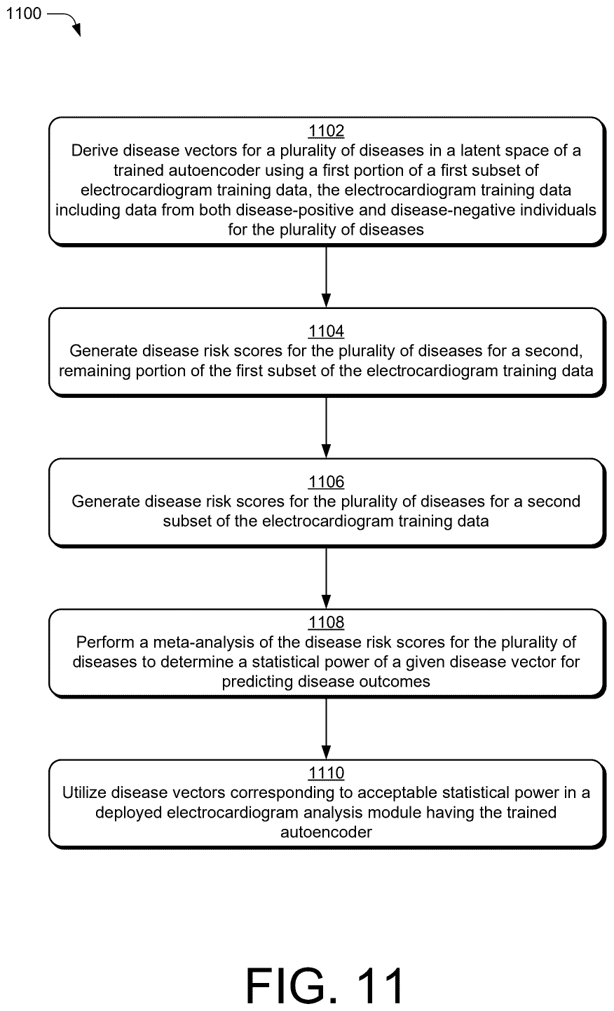

FIG. 11 depicts an example procedure 1100 for deriving and utilizing disease vectors from electrocardiogram data according to one or more implementations. Where appropriate, reference will be made to components previously introduced in FIGS. 1-9.

Disease vectors are derived for a plurality of diseases in a latent space of a trained autoencoder using a first portion of a first subset of electrocardiogram training data, the electrocardiogram training data including data from both disease-positive and disease-negative individuals for the plurality of diseases (block 1102). By way of example, the ECG analysis module 130 may derive the disease vectors by analyzing the ECG encodings 218 generated by the encoder network 214 in the latent space 138. The disease vectors may be derived by analyzing the distribution of ECG encodings 218 in the latent space 138, such as illustrated in FIG. 8. By way of example, the disease-negative data points 804 and the disease-positive data points 806 may cluster differently within the latent space 138. For each disease of the plurality of diseases, a disease vector may be derived between the disease negative centroid 808 and the disease positive centroid 810. This vector, represented as the at least one disease vector 140, illustrates the direction of separation between the disease-negative and disease-positive clusters in the latent space 138.

The at least one disease vector 140 captures the most salient differences between disease-positive and disease-negative ECG characteristics in a high-dimensional space. By deriving these vectors for multiple diseases, the system creates a framework for quantifying the relationship between any given ECG and various disease states or other phenotypes (e.g., age, sex, body mass index, and so forth). This approach leverages the ability of the autoencoder to learn complex patterns or relationships in the ECG data that may not be apparent through traditional ECG analysis methods.

Disease risk scores are generated for the plurality of diseases using a second, remaining portion of the first subset of the electrocardiogram training data (block 1104). By way of example, the statistical modeling algorithm 136 may process the ECG encodings 218 generated from the second portion of the first subset of the electrocardiogram training data in the latent space 138 to generate vector component scores 702 by projecting the ECG encodings 218 onto the derived disease vectors. These vector component scores 702 may be used to calculate disease risk scores for each of the plurality of diseases.

As illustrated in FIG. 9, for example, the vector component score 702 represents the projection of an individual ECG encoding 218 onto a specific disease vector (the at least one disease vector 140) in the latent space 138. The magnitude of this projection quantifies the relationship between a given ECG and the corresponding disease state. The vector component score 702 may be calculated using the angle θ between the ECG encoding 218 and the at least one disease vector 140, as shown in the geometric representation in FIG. 9.

The statistical modeling algorithm 136 may employ various techniques to derive disease risk scores from these vector component scores. These techniques may include logistic regression, Cox proportional hazards models, or other machine learning methods. The resulting disease risk scores (e.g., the at least one disease risk score 116) may include probability scores indicating the likelihood a corresponding patient has a corresponding disease and/or a risk of developing the corresponding disease (e.g., the disease or condition corresponding to the disease vector) within a specified time frame. In some implementations, the disease risk scores may also include estimated time frames for potential disease onset or indications of disease severity. As another example, the risk may be categorized as low, moderate, or high, or using another stratification technique. The disease risk scores enable a quantitative assessment of disease risk based on the ECG data, which may allow for early detection and intervention across a range of medical conditions.

Disease risk scores are generated for the plurality of diseases using a second subset of the electrocardiogram training data (block 1106). By way of example, the external test subset 152 may be evaluated using the same process as in block 1104. This allows for validation of the disease risk prediction on a separate dataset.

In some implementations, the external test subset 152 may comprise ECG data from one or more separate cohorts, often collected from a different institution and/or different patient population than the first subset of electrocardiogram training data. The use of this external dataset may help assess the generalizability of the disease risk prediction model across diverse patient populations and clinical settings.

A meta-analysis of the disease risk scores for the plurality of diseases is performed to determine a statistical power of a given disease vector for predicting disease outcomes (block 1108). By way of example, the statistical modeling algorithm 136 may analyze the disease risk scores generated from both the first and second subsets of the electrocardiogram training data to assess the predictive power of each disease vector across multiple independent cohorts from various healthcare institutions and population databases. The meta-analysis may evaluate the consistency and strength of associations between the ECG-derived disease risk scores and various disease outcomes across these different populations. By way of example, the statistical power of each disease vector may be assessed using metrics such as odds ratios, confidence intervals, and/or p-values. For instance, odds ratios for various conditions may be calculated and compared across datasets. The consistency of these associations across different cohorts may provide evidence for the robustness and generalizability of the disease vectors.

The meta-analysis may also include calculating improvements in discrimination when using ECG vector component scores compared to models without them. By way of example, this may be quantified using differences in area under the curve (AUC) values across different disease categories and datasets. Additionally, the meta-analysis may include perturbation tests to validate the robustness of the associations. This may include randomly reclassifying phenotype labels at different percentages and observing how the statistical significance of associations changes. This process helps demonstrate that the identified associations are not due to chance and are resistant to random noise in the data. The results of this meta-analysis may be used to identify which disease vectors demonstrate the strongest and most consistent predictive power across different populations and datasets.

Disease vectors corresponding to acceptable statistical power are utilized in a deployed electrocardiogram analysis module having the trained autoencoder (block 1110). By way of example, the ECG analysis module 130 may incorporate the disease vectors that demonstrate strong predictive power into the deployed model for real-world applications. The at least one disease vector 140 may be used to generate disease risk scores 116 for new ECG data 126 input into the system, for example.

In this way, the procedure 1100 enables the derivation and validation of disease-specific vectors in the latent space of the autoencoder. As a result, disease (or other phenotype) vectors having strong discriminatory power may be used in the deployed electrocardiogram analysis module for evaluating new, unseen ECG data, including ECG data for which a state is unknown with respect to the one or more of the diseases of the disease vectors.

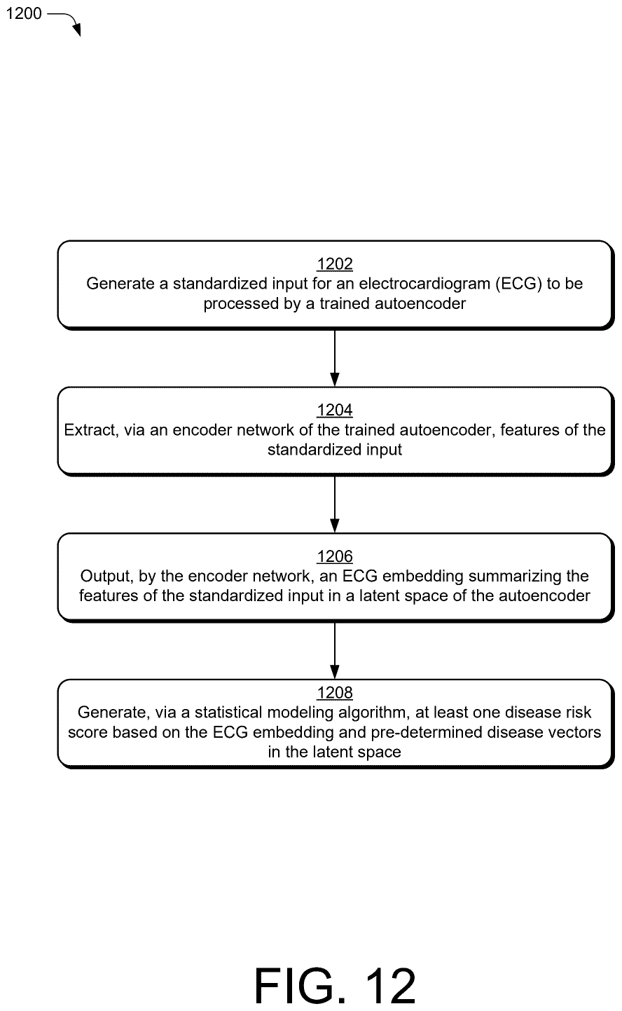

FIG. 12 depicts an example procedure 1200 for processing electrocardiogram data using a machine learning model according to one or more implementations. Where appropriate, reference will be made to components previously introduced in FIGS. 1-9.

A standardized input is generated for an electrocardiogram (ECG) to be processed by a trained autoencoder (block 1202). By way of example, the data preprocessor 132 may generate the standardized input 208 from the ECG input 204, which corresponds to the ECG data 126 recorded from an individual. The ECG input 204, for instance, may be a 12-lead ECG. The standardized input 208 may be processed to have consistent dimensions, sampling rate, and other characteristics to ensure uniform input to the autoencoder. Moreover, data overlays and non-ECG data (e.g., patient information, text) may be removed to generate the standardized input 208.

Features of the standardized input are extracted via an encoder network of the trained autoencoder (block 1204). By way of example, the trained autoencoder (e.g., the deep learning autoencoder 134) may utilize the encoder network 214 to extract features from the standardized input 208. These features may represent various aspects of cardiac electrical activity depicted in the ECG. The encoder network 214 learns to identify and extract relevant features that enable accurate reconstruction of the input ECG. During training, if a particular feature helps to minimize the loss 230, the training may reinforce this feature by adjusting the corresponding weights 220 and biases 222 of the encoder network 214. In contrast, a feature that does not contribute to reducing the loss 230 may not be emphasized in the encoding process.

The encoder network outputs an ECG embedding that summarizes the features of the standardized input in a latent space of the autoencoder (block 1206). By way of example, the encoder network 214 may output the ECG encoding 218, which represents the extracted features within the latent space 138. The ECG encoding 218, for instance, may capture both spatial and temporal information to distill the information of the standardized input 208 into a lower-dimensional, meaningful representation that can be used for downstream tasks like disease risk prediction.

At least one disease risk score is generated via a statistical modeling algorithm based on the ECG embedding and pre-determined disease vectors in the latent space (block 1208). By way of example, the statistical modeling algorithm 136 may process the ECG encoding 218 to generate a vector component score 702 by projecting the ECG encoding 218 onto individual vectors of the pre-determined disease vectors (at least one disease vector 140) in the latent space 138. The vector component score 702 may then be used to determine the at least one disease risk score 116 for the corresponding disease or condition.

The at least one disease risk score 116 may include probability scores indicating the likelihood of the individual having various diseases or risk of developing diseases within specified time frames. Additionally, the at least one disease risk score 116 may include estimated time frames for potential disease onset or indications of disease severity, such as by categorizing the risk as low, moderate, or high. The at least one disease risk score 116 may provide clinicians with a comprehensive assessment of the individual's disease risks, enabling more informed decision-making for patient care and follow-up.

In this way, the procedure 1200 enables use of a trained autoencoder to output accurate disease risk predictions based on ECG data. The ability of the autoencoder to analyze ECG data and provide disease risk predictions may assist clinicians in identifying patients at risk of developing various conditions, even when other clinical indicators appear normal. This approach may enable earlier interventions and more accurate risk stratification across a range of medical conditions.

Having described example procedures in accordance with one or more implementations, consider now an example system and device that can be utilized to implement the various techniques described herein.

### Example System and Device

FIG. 13 illustrates an example system generally at 1300 that includes an example computing device 1302 that is representative of one or more computing systems and/or devices that may implement the various techniques described herein. This is illustrated through inclusion of the computing device 108. The computing device 1302 may be, for example, a server of a service provider, a device associated with a client (e.g., a client device), an on-chip system, and/or any other suitable computing device or computing system.

The example computing device 1302 as illustrated includes a processing system 1304, one or more computer-readable media 1306, and one or more I/O interfaces 1308 that are communicatively coupled, one to another. Although not shown, the computing device 1302 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1304 is representative of functionality to perform one or more operations using hardware. Accordingly, the processing system 1304 is illustrated as including hardware elements 1310 that may be configured as processors, functional blocks, and so forth. This may include implementation in hardware as an application specific integrated circuit or other logic device formed using one or more semiconductors. The hardware elements 1310 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable media 1306 is illustrated as including memory/storage 1312. The memory/storage 1312 represents memory/storage capacity associated with one or more computer-readable media. The memory/storage 1312 may include volatile media (such as random-access memory (RAM)) and/or nonvolatile media (such as read only memory (ROM), Flash memory, optical disks, magnetic disks, and so forth). The memory/storage 1312 may include fixed media (e.g., RAM, ROM, a fixed hard drive, and so on) as well as removable media (e.g., flash memory, a removable hard drive, an optical disc, and so forth). The computer-readable media 1306 may be configured in a variety of other ways as further described below.

Input/output interface(s) 1308 are representative of functionality to allow a user to enter commands and information to computing device 1302, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1302 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1302. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and nonvolatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1302, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1310 and computer-readable media 1306 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1310. The computing device 1302 may be configured to implement particular instructions and/or functions corresponding to the software and/or hardware modules. Accordingly, implementation of a module that is executable by the computing device 1302 as software may be achieved at least partially in hardware, e.g., through use of computer-readable storage media and/or hardware elements 1310 of the processing system 1304. The instructions and/or functions may be executable/operable by one or more articles of manufacture (for example, one or more computing devices 1302 and/or processing systems 1304) to implement techniques, modules, and examples described herein.

The techniques described herein may be supported by various configurations of the computing device 1302 and are not limited to the specific examples of the techniques described herein. This functionality may also be implemented all or in part through use of a distributed system, such as over a “cloud” 1314 via a platform 1316 as described below.

The cloud 1314 includes and/or is representative of a platform 1316 for resources 1318, which are depicted including the computing device 108. The platform 1316 abstracts underlying functionality of hardware (e.g., servers) and software resources of the cloud 1314. The resources 1318 may include applications and/or data that can be utilized while computer processing is executed on servers that are remote from the computing device 1302. The resources 1318 can also include services provided over the Internet and/or through a subscriber network, such as a cellular or Wi-Fi network.

The platform 1316 may abstract resources and functions to connect the computing device 1302 with other computing devices. The platform 1316 may also serve to abstract scaling of resources to provide a corresponding level of scale to encountered demand for the resources 1318 that are implemented via the platform 1316. Accordingly, in an interconnected device example, implementation of functionality described herein may be distributed throughout the system 1300. For example, the functionality may be implemented in part on the computing device 1302 as well as via the platform 1316 that abstracts the functionality of the cloud 1314.

Having discussed example details of the techniques for artificial intelligence enabled disease profiling, consider now the following examples to illustrate additional aspects of the described techniques.

### ECG Recording Device

In at least one implementation, the ECG waveform data (e.g., the ECG signal 120) is recorded with an ECG recording device (e.g., the electrocardiogram system 106). ECG recording devices can comprise a standard ECG recording device, an ambulatory ECG recording device, or both. A standard ECG recording device records the electrical activity of a heart (i.e., ECG waveform data) for a short period of time (e.g., approximately 10 seconds). An ambulatory ECG recording device records the electrical activity of a heart for an amount of time (e.g., greater than 10 seconds).

In an example implementation, the ECG recording device comprises an ambulatory ECG recording device, wherein the electrical activity of the heart is measured for at least 30 seconds, at least one minute, at least five minutes, at least 30 minutes, at least one hour, at least six hours, at least 12 hours, at least one day, at least two days, at least 15 days, at least one month, at least six months, or at least one year. In an example implementation, the standard or ambulatory ECG device comprises a multi-lead surface monitor, a Holter monitor, an event monitor, a patch monitor, an implantable monitor, or a consumer device (e.g., a smartwatch).

A multi-lead surface monitor typically refers to a standard surface ECG monitor. A standard surface monitor comprises electrodes (e.g., the ECG sensor(s) 122) placed on the surface of a subject. A standard surface ECG monitor can include one to 12-leads placed at different locations across the subject. The leads capture the differences in electrical activity measured by the electrodes. Electrodes are typically placed at the right arm (RA), left arm (LA), left leg (LL), right leg (RL) and V1-V6 along the chest (precordial). The 12 leads comprise six limb (or extremity) leads and six chest leads. The six limb leads correspond to the RA, LA, LL, and RL electrodes. The six limb leads comprise three bipolar leads (i.e., I, II, and III) and six unipolar leads (i.e., aV_{R}, aV_{L}, and aV_{F}). Lead I corresponds to both electrodes LA and RA, lead II corresponds to both electrodes LL and RA and lead III corresponds to both LL and LA. Leads aV_{R}, aV_{L}, and aV_{F} individually correspond to any one of electrodes RA, LA, LL, and RL and are assigned depending on the need during monitoring. The six chest leads correspond to the six chest electrodes. Any two or more electrodes can establish a 1- to 12-lead system. For example, in a three-lead system, only three electrodes are placed, typically at the RA, LA, and LL locations on the surface of a subject. The three electrodes correspond to three bipolar leads: I, II, and III.

Other ECG recording devices use different combinations of electrodes to measure the heart's electrical activity. A Holter monitor is a portable device comprising two or more (e.g., five to seven) leads and continuously records every heartbeat. An event monitor, on the other hand, generally does not attempt to record every heartbeat. In some instances, the event monitor is worn continuously during the course of monitoring. In some instances, the event monitor is worn when symptoms occur. Event monitors worn continuously can comprise automatic symptom detection and only record if a symptom is detected, at least in some implementations. Event monitors comprising automatic symptom detection can include manual recording as well. Event monitors can be symptom monitors or memory looping monitors. One differentiating feature between symptom and memory looping monitors is memory looping monitors record the electrical activity of the heart for some time before the memory looping monitor is activated.

Patch monitors are self-contained monitors attached directly to a subject. All the components, including the leads, are contained within a single device. Typically, patch monitors are comprised of an adhesive that attaches directly to a subject. Patch monitors generally record substantially all electrical activity of the heart while monitoring. Some patch monitors can wirelessly transmit recorded electrical activity of the heart. Implantable monitors, or implantable loop monitors, are inserted under the skin of a subject. Typically, implantable monitors record the electrical activity of the heart continuously during monitoring. During operation, the recorded data is periodically transmitted wirelessly to an exterior device for further processing. In some instances, a patch monitor or implantable monitor can use leadless technology.

Consumer devices can comprise any monitor previously mentioned but are designed for personal use. Consumer devices can also comprise wearable mobile devices (e.g., smartwatches). Consumer device monitors can comprise a single device or multiple devices worn on or attached to the body to monitor and record the electrical activity of the heart.

In an implementation, “leadless” ECG recording devices are used. Leadless ECG recording devices use wireless technology and at least two points of measurement to record the heart's electrical activity. In an implementation, leadless ECG recording devices record measurements from two points of the body (e.g., right arm/left arm or right leg/left leg). The two points may be on the surface of a subject (e.g., body) opposite each other where the heart is situated between the two points.

### ECG Waveform

ECG waveform data (e.g., the ECG signal 120) can comprise any electrical activity of a subject's heart. This electrical activity can be picked up on the skin and transmitted throughout the body. The direction of the ECG waveform data (i.e., direction of a deflection, upward or downward), can vary depending on whether the electrical activity is going towards or away from a lead. This electrical activity's typical characteristic feature comprises three waves: the P wave, the QRS wave complex, and the T wave.

The P wave typically presents as a slight deflection and represents atrial depolarization. The QRS wave complex comprises multiple presentations. For example, if the wave immediately after the P wave deflects downwards, the first wave in the QRS wave complex is a Q wave. If the wave immediately after the P wave deflects upward, then the first wave in the QRS wave complex is an R wave. The Q wave can comprise various sizes. For example, a small Q wave can correspond to depolarization of the interventricular septum, while significant and wide Q waves can correspond to an old myocardial infarction. In general, Q waves correspond to breathing and are small and thin. R waves are typically the largest wave and correspond to the depolarization of the main mass of the ventricles. S waves represent the final depolarization of the ventricles. The T wave represents ventricular repolarization. In an example implementation, the ECG waveform data can further comprise U waves, which are small waves following T waves.

The typical characteristic features of a heart's electrical activity may further comprise characteristic features representing the relationship between the three waves. These features can comprise segments or intervals of the wave. A segment is a region between two waves, while an interval is a duration of time between waves and includes a segment. For example, the PR interval represents a measure of time between the P wave's first deflection and the QRS complex's first deflection and includes the PR segment. The ST interval represents a measure of time between the end of the QRS complex and the end of the T wave including the ST segment. It characterizes the period of zero potential between ventricular depolarization and repolarization. The QT interval is the time from the start of the Q wave to the end of the T wave and is inversely correlated with heart rate. The QTc-interval is a QT-interval corrected for heart rate. Various formulae to correct QT-intervals are well known in the art and will not be mentioned herein for brevity. Additional features can include the J point, which is the junction between the termination of the QRS complex and the beginning of the ST segment.

Every characteristic feature of ECG waveform data was not mentioned for brevity. One of ordinary skill in the art would immediately recognize features not mentioned herein can be included in ECG waveform data.

### Reconstructed Waveform

Reconstructed waveform data is transformed input data, output from a machine learning model. The waveform data can be transformed into a single- or multi-dimension data structure and can include vector components. In general, the machine learning model is trained to transform ECG waveform data into a new, lower-dimensional representation, examples of which are described herein. The new representation of the ECG waveform data encodes intrinsic patterns and characteristics learned from training a machine learning model against ECG waveforms of subjects with and without any one or more diseases. The transformation into the new representation can include any form of embedding or encoding. The machine learning model may be tuned such that an ECG waveform is reconstructed from the new representation. Moreover, the new representation may represent the health of a subject with respect to any one or more diseases (e.g., whether the subject has any of the one or more diseases). In an implementation, the ECG waveform data is high-dimensional in form and is transformed into a lower-dimensional form, e.g., within the latent space 138. This can reduce noise and remove uncorrelated features from the high-dimensional data and result in lower-dimensional data that can be enriched for correlated features.

By way of example, the new representation (e.g., the lower-dimensional data) is projected against one or more disease vectors, generating one or more disease risk scores (e.g., the at least one disease risk score 116). The disease risk score is user-comprehensible information, including a disease score output of one or more etiology diagnoses of disease.

### Disease Vectors

A disease vector (e.g., the at least one disease vector 140) is a single- or multi-dimension data structure and can include vector components. A disease vector, in general, is a representation of one or more types of measurements of multiple subjects with a disease and one or more types of the same measurements of multiple subjects without said disease as well as the space between them. In an implementation, a disease vector is data that includes a representation of subjects with a disease, a representation of those without the disease, and the information connecting those two groups/groupings. In an implementation, a disease vector includes a first generation of one or more of the same measurements, which are indicators of a disease from the health data of subjects with the disease. The disease vector can also include a second generation of one or more of the same measurements from the health data of subjects without the disease. The results from the first generation are grouped, and the results from the second generation are grouped, forming a first and second grouping. An indicator of the space and/or position between the first and second groups can then be generated, creating a disease vector, as further described herein, e.g., with respect to FIG. 8.

In an implementation, the disease vector is generated from a machine learning model. In an implementation, the disease vector generated from the machine learning model is trained on health data. In an implementation, the disease vector generated from the machine learning model is trained on ECG waveform data. For example, the ECG waveform data from more than one subject with a disease and without a disease are recorded. The recorded ECG waveform data is then used as input to train a machine learning model or is used as input data in a trained machine learning model. In the instance of training a machine learning model, the machine learning model is trained using data from two groups: those with and those without a disease. In at least some implementations, embeddings from the two groups form distinct groupings with respect to each other within the latent space 138. The corresponding groupings are then used to form the disease vector for the trained machine learning model.

In an implementation, the disease vector generated from the machine learning model connects two centroids: a disease-positive centroid and a disease-negative centroid. For example, the output from a machine learning model based on the input of waveform data from subjects with a disease can form a first set of points in the latent space 138, with the disease-positive centroid being the mean position of the first set of points. The output from a machine learning model based on the input of waveform data from subjects without the disease can form a second set of points in the latent space 138, with the disease-negative centroid being the mean position of the second set of points. A line can then be made between the disease-positive centroid and the disease-negative centroid, thus forming a disease vector for a given disease.

### Disease Risk Score

A disease risk score (also referred to as a vector component score) is a representation/indicator/biomarker of a subject's risk for having and/or developing a disease. The disease risk score is generated by projecting an embedding generated from waveform data onto a disease vector. The disease risk score, therefore, is a quantitative measure of a subject's risk for having or developing a disease and can be a biomarker for the disease. In an implementation, the disease risk score is a scalar value. In an implementation, the disease risk score is a vector.

A disease risk score can indicate the presence of a disease by having a value or values within, above, or below a certain threshold. For example, a value or values above or below a threshold can indicate ECG waveform data includes an irregularity caused by a disease. A threshold can be a single value or a range of values. A threshold range can include any width relative to the possible values for the disease risk score. For example, the threshold range can extend from any positive values to any negative values. The threshold range can vary by any whole number (i.e., integer value) or decimal number.

Alternatively, or in addition, a disease risk score can indicate whether a subject is developing or has a likelihood of developing a disease. For example, a value or values of a disease risk score can be near a defined threshold for having a disease. To be near a defined threshold can include a single value or range of values. For example, the value or values for developing or the likelihood of developing a disease can be a single value immediately following the threshold for having a disease or a range of values starting from the top and/or bottom of a threshold and extending above and/or below, respectively, from that threshold. In an implementation, the value or values for developing or the likelihood of developing a disease extend on either side of the value or threshold for having a disease.

In an example implementation, the disease risk score indicates whether a subject is developing or has a likelihood of developing a disease and is defined similarly to or the same as indicating whether a subject has a disease. In an implementation, there is more than one threshold for having a disease. The more than one threshold includes a first threshold, which indicates the subject is developing or has the highest likelihood of developing a disease. The more than one threshold can include a last threshold, which can be one or more than one threshold on either side of the first threshold, and indicates the subject is not developing or has the lowest likelihood of developing a disease. The more than one threshold can include any number of thresholds between the first threshold and a final threshold, the final threshold can be the last threshold. Any threshold that is not the first threshold can be a threshold for indicating a subject is slower to develop or is less likely to develop a disease than the first threshold, at least in some implementations.

### Disease Risk Decile

In an implementation, the disease risk score can be defined by deciles. A decile includes any division of a range of values by 1/10. For example, the range of disease risk scores can be from 100 (having, or representing a highest likelihood of having, a disease) to 1 (not having, or representing a lowest likelihood of having, a disease). The deciles between 1 and 100 can take any form. As an example, the decile can be evenly spaced between 100 and 1 and include every 10 values (e.g., 10+10+10+10+10+10+10+10+10+10=100). In another example, the decile can be unevenly spaced between 100 and 1, and the values of each decile are independently selected based on the risk of disease such that the total from each 10 deciles equals the total number of values (e.g., 10+20+7+12+16+5+4+6+11+9=100). The deciles can be selected such that the decile including 100 is the largest decile and the deciles get progressively smaller towards 1 (e.g., 20+16+12+11+10+9+7+6+5+4=100). The deciles can be selected such that the decile including 1 is the largest decile and the deciles get progressively smaller towards 100. The decile can be selected such that each decile is of varying sizes (e.g., 10+20+7+12+16+5+4+6+11+9=100). It is understood the construction of deciles can also take the form of percentages or ratios and that using values of 100 and 10 are provided by way of example.

In an implementation, a decile is developed using information about one or more subjects including health information from a subject. For example, the health information can include one or more of age, sex, race, or heart-related data (e.g., heart rate, blood pressure, ECG). In an implementation, a decile is developed using information about one or more subjects including the output from a machine learning model. In an implementation, a decile is developed using information about one or more subjects including reconstructed waveform data. In an implementation, a decile is developed using information about one or more subjects including a disease risk score.

In general, a decile can be developed by grouping one or more feature (e.g., health information, machine learning model output, reconstructed waveform data, disease risk score, etc.) from one or more subjects. The threshold based on the combined values from the one or more features from the one or more subjects, for example, can be combined using an average, a median, a mean, a mode, a mid-range, a centroid, or any combination thereof. In example implementations, a decile is dependent on the age of the one or more subjects. For example, when developing a decile, the one or more subjects have a singular age (e.g., 65) or a range of ages (e.g., 60-70).

In an implementation, a top or a highest decile can be developed by grouping one or more feature (e.g., health information, machine learning model output, reconstructed waveform data, disease risk score, etc.) from one or more subjects who have a disease and creating a threshold based on the combined values from the one or more features from the one or more subjects who have the disease. By way of another example, a bottom or a lowest decile can be created by grouping one or more feature (e.g., health information, machine learning model output, reconstructed waveform data, disease risk score, etc.) from one or more subjects who do not have the disease and/or has never developed the disease and creating the threshold based on the combined values from the one or more features from the one or more subjects who do not have the disease.

In an implementation, a decile between the top or the highest decile and the bottom or the lowest decile can be created by grouping one or more feature (e.g., health information, machine learning model output, reconstructed waveform data, disease risk score, etc.) from the one or more subjects before they detectably (i.e., measurably) developed a disease or after a subject detectably (i.e., measurably) no longer has the disease. The one or more features can come from an immediate time frame right before a subject developed a disease or a later time frame some time before a subject developed the disease. The one or more features can come from an immediate time frame right after a subject no longer has the disease (e.g., days, weeks, and/or months) or a later time frame some time after the subject no longer has the disease (e.g., years and/or decades).

In an implementation, the decile immediately following the top or highest decile or the bottom or lowest decile can use features from an immediate time frame. In an implementation, deciles not immediately following the top or highest decile or the bottom or lowest decile can use features from a later time frame. It is understood that the time frame used to develop a decile can vary by disease. For example, a disease that develops quickly can use more than one decile developed from an immediate time frame. A disease that develops slowly, for example, can use more than one decile developed from a later time frame. In general, a combination of one or more immediate time frames and one or more later time frames can be used to develop the deciles for a disease. In at least one variation, the deciles are not based on time frames but are based on a determined disease risk score and/or disease risk score range.

In an implementation, one or more disease risk scores comprise a risk decile for one or more corresponding diseases, wherein a top risk decile indicates the highest risk of having or developing a disease, and a bottom risk decile indicates the lowest risk of having or developing a disease. For example, if the disease risk score is from 100 to 1 and if the deciles are constructed as [20+16+12+11+10+9+7+6+5+4=100], then the top decile would be a value in the range of 20 and would include the numbers 100-81 and the bottom decile would be a value in the range of 4 and include the numbers 4-1.

In at least one variation, rather than defining the disease risk score in terms of deciles, other divisions are possible, such as tertiles, quartiles, quintiles, and the like. Binary outputs are also possible. Moreover, the disease risk score may indicate a probability in addition to or as an alternative to stratifying patients into risk categories.

### Disease and Disease Etiologies

The diseases and/or disease etiologies corresponding to the disease risk score can be independently selected from the group consisting of heart disease and non-cardiac disease. By way of example, the machine learning model produces a latent space embedding, and the latent space embedding is projected against a disease vector to generate a disease risk score. The disease risk score determines whether a subject is suffering from or can suffer from a disease from at least one of at least the following disease categories: circulatory system, respiratory, endocrine/metabolic, digestive, genitourinary, hematopoietic, infectious diseases, injuries and poisonings, mental disorders, musculoskeletal, neoplasms, neurological, pregnancy complications, congenital anomalies, dermatologic, sense organs, and symptoms. Examples of such diseases include hypertension, essential hypertension (e.g., primary hypertension that does not have a known secondary cause), cardiomyopathy, obesity, diabetes mellitus, disorders of fluid/electrolyte/acid-base balance, pulmonary congestion and hypostasis, cardiac defibrillator in situ, tobacco use disorder, fever of unknown origin, non-alcoholic liver disease, bundle branch block, left bundle branch block, right bundle branch block, congenital cardiac anomalies, decubitus ulcer, chronic ulcer of leg or foot, portal hypertension, acute and subacute necrosis of liver, cachexia, alkalosis, type 1 diabetes with peripheral circulatory disorders, nephritis and nephropathy, renal dialysis, acute renal failure, end stage renal disease, anemia in chronic kidney disease, gram positive septicemia, bacteremia, septicemia, Hodgkin's disease, coma, peripheral autonomic neuropathy, obstructive chronic bronchitis, respiratory failure, emphysema, and cardiogenic shock.

For example, common ECG parameters associated with cardiovascular mortality are the P wave (duration, interatrial block, and deep terminal negativity of the P wave in V1), prolonged QT and Tpeak-Tend intervals, QRS duration and fragmentation, bundle branch block, ST segment depression and elevation, T waves (inverted, T wave axes), spatial angles between QRS and T vectors, premature ventricular contractions, and ECG hypertrophy criteria. In analyzing the waveform data of a subject, the machine learning model can reconstruct the ECG waveform data and, after projecting it onto a disease vector, can result in a probable diagnosis of a disease or an etiology of a disease.

In an implementation, the disease is any of those selected from the groupings in Table 2. In an implementation, the disease is any of those selected from Table 3, or any combination thereof.

In an implementation, the one or more diseases are a rare disease. In an implementation, a rare disease can be a rare cardiac disease. In an implementation, rare cardiac disease includes Brugada syndrome, Cardiomyopathy, Ebstein anomaly, Tetralogy of Fallot, Cardiac amyloidosis, Chagas disease, Eisenmenger syndrome, Long QT syndrome, Atrioventricular canal defect, Prinzmetal Angina, or Torsades de Pointes.

### Monitoring

In an implementation, the methods, systems, devices, and products described herein are used to monitor the development or course of a disease. For example, a disease can be first detected, with the methods described herein or via another method, and the method is repeated over a course of time to monitor/observe the development or course of a disease. Using the methods described herein to monitor the development or course of a disease gives the advantage of identifying changes in the development or course of a disease that cannot be manually determined (e.g., by human review) of the ECG waveform data of a subject. Treatments can then be altered or modified according to the development or course of the disease monitored by the methods described herein.

In an implementation, the methods described herein are performed after the subject has received one or more treatments. For example, if a subject is first diagnosed with a disease, then the subject is provided a treatment for said disease, and then the subject is monitored with a method described herein. The output from the method can then indicate the efficacy of the treatment on the disease. In an implementation, the method is repeated at multiple time points over the course of treatment. For example, the method can be performed any time the treatment is administered. In an implementation, the method is performed periodically. For example, the method is performed every hour, every day, every month, every year, every five years, every ten years, or any combination thereof. The method can be performed every other hour, every other day, every other week, every other month, every other year, every other five years, every other ten years, or any combination thereof. The method can be performed in any combination previously described for monitoring a subject.

### Machine Learning

Machine learning is a field of study within artificial intelligence that allows computers to learn functional relationships between inputs and outputs without being explicitly programmed. Machine learning involves a module comprising algorithms that can learn from existing data by analyzing, categorizing, and/or identifying the data. Such machine learning algorithms may operate by first constructing a model from training data to make predictions, decisions, and/or reconstructions expressed as outputs. In an implementation, the training data includes data for one or more identified features and one or more outcomes, for example waveform data and reconstructed waveform data, respectively. Although example implementations are presented with respect to a few machine learning algorithms, the principles presented herein can be applied to other machine learning algorithms.

Data supplied to and/or transformed by a machine learning algorithm can be considered a feature, which can be described as an individual measurable property of a phenomenon being observed. The performance of a machine learning algorithm in pattern recognition, classification, regression, representation learning, and/or reconstruction is highly dependent on choosing informative, discriminating, and independent features. Features can comprise numerical data, categorical data, time-series data (e.g., time-series ECG waveform data), strings, graphs, or images. By way of example, time-series ECG waveform data can include PR, QRS voltages, QT, and QTc-intervals, wherein the characteristic features from the QRS voltage can comprise the Q, R, or S signal or any combination thereof. In one example implementation, reconstructing waveform data comprises reconstructing one or more of these characteristic features.

### Embedding

In one example, the machine learning module can use embedding to provide a lower dimensional representation, such as a vector, of features to organize them based off respective similarities. In some situations, these vectors can become massive. In the case of massive vectors, particular values can become very sparse among a large number of values (e.g., a single instance of a value among 50,000 values). Because such vectors are difficult to work with, reducing the size of the vectors, in some instances, is necessary. A machine learning module can learn the embeddings along with the model parameters. In an implementation, features such as age, sex, heart rate, PR, QRS voltages, QT, QTc-intervals, wherein the characteristic features from the QRS voltage can comprise the Q, R, or S signal or any combination thereof, can be mapped to a lower-dimensional latent space. In an implementation, embedded semantic meanings are utilized. Embedded semantic meanings are values of respective similarity. For example, the distance between two vectors, in vector space, can imply two values located elsewhere with the same distance are categorically similar or dissimilar. Embedded semantic meanings can be used with similarity analysis to rapidly return similar values. In an implementation, age, sex, heart rate, PR, QRS voltages, QT, QTc-intervals, wherein the characteristic features from the QRS voltage can comprise the Q, R, or S signal or any combination thereof is embedded. In an implementation, waveform data is embedded along with corresponding phenotypical data from one or more subject. Projections along the vector components of the waveform data and the corresponding phenotypical data can be used in reconstructing waveform data. In an implementation, the methods herein are developed to identify meaningful portions of the vector and extract semantic meanings between that space.

### Training Methods

In an implementation, the machine learning module can be trained using techniques such as unsupervised, supervised, semi-supervised, reinforcement learning, transfer learning, incremental learning, curriculum learning techniques, and/or learning to learn. Training typically occurs after selection and development of a machine learning module and before the machine learning module is operably in use. In one aspect, the training data used to teach the machine learning module can comprise input data such as waveform data and the respective target output data such as reconstructed waveform data.

In an example implementation, unsupervised learning is implemented. Unsupervised learning can involve providing all or a portion of unlabeled training data to a machine learning module. The machine learning module can then determine one or more outputs implicitly based on the provided unlabeled training data. In an example implementation, supervised learning is implemented. Supervised learning can involve providing all or a portion of labeled training data to a machine learning module, with the machine learning module determining one or more outputs based on the provided labeled training data, and the outputs are either accepted or corrected depending on the agreement to the actual outcome of the training data. In some examples, supervised learning of machine learning system(s) can be governed by a set of rules and/or a set of labels for the training input, and the set of rules and/or set of labels can be used to correct inferences of a machine learning module.

In one example implementation, semi-supervised learning is implemented. Semi-supervised learning can involve providing all or a portion of training data that is partially labeled to a machine learning module. During semi-supervised learning, supervised learning is used for a portion of labeled training data, and unsupervised learning is used for a portion of unlabeled training data. In one example implementation, reinforcement learning is implemented. Reinforcement learning can involve first providing all or a portion of the training data to a machine learning module and as the machine learning module produces an output, the machine learning module receives a “reward” signal in response to a correct output. Typically, the reward signal is a numerical value, and the machine learning module is developed to maximize the numerical value of the reward signal. In addition, reinforcement learning can adopt a value function that provides a numerical value representing an expected total of the numerical values provided by the reward signal over time.

In one example implementation, transfer learning is implemented. Transfer learning techniques can involve providing all or a portion of a first training data to a machine learning module, then, after training on the first training data, providing all or a portion of a second training data. In an implementation, a first machine learning module can be pre-trained on data from one or more computing devices. The first trained machine learning module is then provided to a computing device, where the computing device is intended to execute the first trained machine learning model to produce an output. Then, during the second training phase, the first trained machine learning model can be additionally trained using additional training data, where the training data can be derived from kernel and non-kernel data of one or more computing devices. This second training of the machine learning module and/or the first trained machine learning model using the training data can be performed using either supervised, unsupervised, or semi-supervised learning. In addition, it is understood that transfer learning techniques can involve one, two, three, or more training attempts. Once the machine learning module has been trained on at least the training data, the training phase can be completed. The resulting trained machine learning model can be utilized as the at least one of trained machine learning module.

In one example implementation, incremental learning is implemented. Incremental learning techniques can involve providing a trained machine learning module with input data that is used to continuously extend the knowledge of the trained machine learning module. Another machine learning training technique is curriculum learning, which can involve training the machine learning module with training data arranged in a particular order, such as providing relatively easy training examples first, then proceeding with progressively more difficult training examples. As the name suggests, difficulty of training data is analogous to a curriculum or course of study at a school.

In one example implementation, learning to learn is implemented. Learning to learn, or meta-learning, comprises, in general, two levels of learning: quick learning of a single task and slower learning across many tasks. For example, a machine learning module is first trained and comprises of a first set of parameters or weights. During or after operation of the first trained machine learning module, the parameters or weights are adjusted by the machine learning module. This process occurs iteratively on the success of the machine learning module. In another example, an optimizer, or another machine learning module, is used wherein the output of a first trained machine learning module is fed to an optimizer that constantly learns and returns the final results. Other techniques for training the machine learning module and/or trained machine learning module are possible as well.

In some examples, after the training phase has been completed but before producing predictions expressed as outputs, a trained machine learning module can be provided to a computing device where a trained machine learning module is not already resident, in other words, after training phase has been completed, the trained machine learning module can be downloaded to a computing device. For example, a first computing device storing a trained machine learning module can provide the trained machine learning module to a second computing device. Providing a trained machine learning module to the second computing device can comprise one or more of communicating a copy of trained machine learning module to the second computing device, making a copy of trained machine learning module for the second computing device, providing access to trained machine learning module to the second computing device, and/or otherwise providing the trained machine learning system to the second computing device. In an implementation, a trained machine learning module can be used by the second computing device immediately after being provided by the first computing device. In some examples, after a trained machine learning module is provided to the second computing device, the trained machine learning module can be installed and/or otherwise prepared for use before the trained machine learning module can be used by the second computing device.

After a machine learning model has been trained, it can be used to output, estimate, infer, predict, generate, or determine; for simplicity these terms will collectively be referred to as results. A trained machine learning module can receive input data and operably generate results. As such, the input data can be used as an input to the trained machine learning module for providing corresponding results to kernel components and non-kernel components. For example, a trained machine learning module can generate results in response to requests. In an implementation, a trained machine learning module can be executed by a portion of other software. For example, a trained machine learning module can be executed by a result daemon to be readily available to provide results upon request.

In an implementation, a machine learning module and/or trained machine learning module can be executed and/or accelerated using one or more computer processors and/or on-device co-processors. Such on-device co-processors can speed up training of a machine learning module and/or generation of results. In some examples, a trained machine learning module can be trained, reside, and execute to provide results on a particular computing device, and/or otherwise can make results for the particular computing device.

Input data can include data from a computing device executing a trained machine learning module and/or input data from one or more computing devices. In an implementation, a trained machine learning module can use results as input feedback. A trained machine learning module can also rely on past results as inputs for generating new results. In an implementation, input data can comprise waveform data and, when provided to a trained machine learning module, results in output data such as reconstructed waveform data.

### Algorithms

Different machine learning algorithms have been contemplated to carry out the implementations discussed herein. For example, linear regression (LiR), logistic regression (LoR), Bayesian networks (for example, naive-bayes), random forest (RF) (including decision trees), neural networks (NN) (also known as artificial neural networks), matrix factorization, a hidden Markov model (HMM), support vector machines (SVM), K-means clustering (KMC), K-nearest neighbor (KNN), a suitable statistical machine learning algorithm, and/or a heuristic machine learning system for classifying or evaluating reconstructed waveform data.

### Linear Regression (LiR)

In one example implementation, linear regression machine learning is implemented. LiR is typically used in machine learning to predict a result through the mathematical relationship between an independent and dependent variable, such as waveform data and reconstructed waveform data, respectively. A simple linear regression model would have one independent variable (x) and one dependent variable (y). A representation of an example mathematical relationship of a simple linear regression model would be y=mx+b. In this example, the machine learning algorithm tries variations of the tuning variables m and b to optimize a line that includes all the given training data.

The tuning variables can be optimized, for example, with a cost function. A cost function takes advantage of the minimization problem to identify the optimal tuning variables. The minimization problem assumes the optimal tuning variable will minimize the error between the predicted outcome and the actual outcome. An example cost function can comprise summing all the square differences between the predicted and actual output values and dividing them by the total number of input values, and results in the average square error.

To select new tuning variables to reduce the cost function, the machine learning module can use, for example, gradient descent methods. An example gradient descent method comprises evaluating the partial derivative of the cost function with respect to the tuning variables. The sign and magnitude of the partial derivatives indicate whether the choice of a new tuning variable value will reduce the cost function, thereby optimizing the linear regression algorithm. A new tuning variable value is selected depending on a set threshold. Depending on the machine learning module, a steep or gradual negative slope is selected. Both the cost function and gradient descent can be used with other algorithms and modules mentioned throughout. For the sake of brevity, both the cost function and gradient descent are well known in the art and are applicable to other machine learning algorithms and may not be mentioned with the same detail.

LiR models can have many levels of complexity comprising one or more independent variables. Furthermore, in an LiR function with more than one independent variable, each independent variable can have the same one or more tuning variables or each, separately, can have their own one or more tuning variables. The number of independent variables and tuning variables will be understood to one skilled in the art for the problem being solved. In an implementation, waveform data are used as the independent variables to train a LiR machine learning module, which, after training, is used to estimate, for example, reconstructed waveform data.

### Logistic Regression (LoR)

In one example implementation, logistic regression machine learning is implemented. Logistic Regression, often considered a LiR type model, is typically used in machine learning to classify information, such as waveform data into categories such as reconstructed waveform data. LoR takes advantage of probability to predict an outcome from input data. However, what makes LoR different from a LiR is that LoR uses a more complex logistic function, for example a sigmoid function. In addition, the cost function can be a sigmoid function limited to a result between 0 and 1. For example, the sigmoid function can be of the form f(x)=1/(1+e^{-x}), where x represents some linear representation of input features and tuning variables. Similar to LiR, the tuning variable(s) of the cost function are optimized (typically by taking the log of some variation of the cost function) such that the result of the cost function, given variable representations of the input features, is a number between 0 and 1, preferably falling on either side of 0.5. As described in LiR, gradient descent can also be used in LoR cost function optimization and is an example of the process. In an implementation, waveform data are used as the independent variables to train a LoR machine learning module, which, after training, is used to estimate, for example, reconstructed waveform data.

### Bayesian Network (BN)

In one example implementation, a Bayesian Network is implemented. BNs are used in machine learning to make predictions through Bayesian inference from probabilistic graphical models. In BNs, input features are mapped onto a directed acyclic graph forming the nodes of the graph. The edges connecting the nodes contain the conditional dependencies between nodes to form a predictive model. For each connected node the probability of the input features resulting in the connected node is learned and forms the predictive mechanism. The nodes can comprise the same, similar or different probability functions to determine movement from one node to another. The nodes of a Bayesian network are conditionally independent of its non-descendants given its parents, thus satisfying a local Markov property. This property affords reduced computations in larger networks by simplifying the joint distribution.

There are multiple methods to evaluate the inference, or predictability, in a BN but only two are mentioned for demonstrative purposes. The first method involves computing the joint probability of a particular assignment of values for each variable. The joint probability can be considered the product of each conditional probability and, in some instances, comprises the logarithm of that product. The second method is Markov chain Monte Carlo (MCMC), which can be implemented when the sample size is large. MCMC is a well-known class of sample distribution algorithms and will not be discussed in detail herein.

The assumption of conditional independence of variables forms the basis for Naïve Bayes classifiers. This assumption implies there is no correlation between different input features. As a result, the number of computed probabilities is significantly reduced as well as the computation of the probability normalization. While independence between features is rarely true, this assumption exchanges reduced computations for less accurate predictions, however the predictions are reasonably accurate. In an implementation, waveform data are mapped to the BN graph to train the BN machine learning module, which, after training, is used to estimate reconstructed waveform data.

### Random Forest (RF)

In one example implementation, random forest is implemented. RF consists of an ensemble of decision trees producing individual class predictions. The prevailing prediction from the ensemble of decision trees becomes the RF prediction. Decision trees are branching flowchart-like graphs comprising of the root, nodes, edges/branches, and leaves. The root is the first decision node from which feature information is assessed and from it extends the first set of edges/branches. The edges/branches contain the information of the outcome of a node and pass the information to the next node. The leaf nodes are the terminal nodes that output the prediction. Decision trees can be used for both classification as well as regression and is typically trained using supervised learning methods. Training of a decision tree is sensitive to the training data set. An individual decision tree can become over or under-fit to the training data and result in a poor predictive model. Random forest compensates by using multiple decision trees trained on different data sets. In an implementation, waveform data are used to train the nodes of the decision trees of a RF machine learning module, which, after training, is used to estimate reconstructed waveform data.

### Gradient Boosting

In an example implementation, gradient boosting is implemented. Gradient boosting is a method of strengthening the evaluation capability of a decision tree node. In general, a tree is fit on a modified version of an original data set. For example, a decision tree is first trained with equal weights across its nodes. The decision tree is allowed to evaluate data to identify nodes that are less accurate. Another tree is added to the model and the weights of the corresponding underperforming nodes are then modified in the new tree to improve their accuracy. This process is performed iteratively until the accuracy of the model has reached a defined threshold or a defined limit of trees has been reached. Less accurate nodes are identified by the gradient of a loss function. Loss functions must be differentiable such as a linear or logarithmic functions. The modified node weights in the new tree are selected to minimize the gradient of the loss function. In an example implementation, a decision tree is implemented to determine reconstructed waveform data and gradient boosting is applied to the tree to improve its ability to accurately determine the reconstructed waveform data.

### Neural Networks (NN)

In one example implementation, Neural Networks are implemented. NNs are a family of statistical learning models influenced by biological neural networks of the brain. NNs can be trained on a relatively large dataset (e.g., 50,000 or more) and used to estimate, approximate, or predict an output that depends on a large number of inputs/features. NNs can be envisioned as so-called “neuromorphic” systems of interconnected processor elements, or “neurons”, and exchange electronic signals, or “messages”. Similar to the so-called “plasticity” of synaptic neurotransmitter connections that carry messages between biological neurons, the connections in NNs that carry electronic “messages” between “neurons” are provided with numeric weights that correspond to the strength or weakness of a given connection. The weights can be tuned based on experience, making NNs adaptive to inputs and capable of learning. For example, an NN for diagnosing reconstructed waveform data is defined by a set of input neurons that can be given input data such as waveform data. The input neuron weighs and transforms the input data and passes the result to other neurons, often referred to as “hidden” neurons. This is repeated until an output neuron is activated. The activated output neuron produces a result. In an implementation, waveform data are used to train the neurons in a NN machine learning module, which, after training, is used to estimate reconstructed waveform data.

### Convolutional Autoencoder (CAE)

In an implementation, a convolutional autoencoder (CAE) is implemented. A CAE is a type of neural network and comprises, in general, two main components. First, the convolutional operator that filters an input signal to extract features of the signal. Second, an autoencoder that learns a set of signals from an input and reconstructs the signal into an output. By combining these two components, the CAE learns the optimal filters that minimize reconstruction error resulting in an improved output. CAEs are trained to learn filters capable of feature extraction that can be used to reconstruct the input. Generally, convolutional autoencoders implement unsupervised learning. In an implementation, the convolutional autoencoder is a variational convolutional autoencoder. In an implementation, features from waveform data are used as an input signal into a CAE, which reconstructs that signal into an output, such as reconstructed waveform data.

### Deep Learning

In an implementation, deep learning is implemented. Deep learning expands the neural network by including more layers of neurons. A deep learning module is characterized as having three “macro” layers: (1) an input layer which takes in the input features, and fetches embeddings for the input, (2) one or more intermediate (or hidden) layers which introduces nonlinear neural net transformations to the inputs, and (3) a response layer which transforms the final results of the intermediate layers to the prediction. In an implementation, waveform data are used to train the neurons of a deep learning module, which, after training, is used to estimate reconstructed waveform data.

### Recurrent Neural Network (RNN)

In an example implementation, a recurrent neural network is implemented. RNNs are a class of NNs further attempting to replicate the biological neural networks of the brain. RNNs comprise of delay differential equations on sequential data or time series data to replicate the processes and interactions of the human brain. RNNs have “memory” wherein the RNN can take information from prior inputs to influence the current output. RNNs can process variable length sequences of inputs by using their “memory” or internal state information. Where NNs can assume inputs are independent from the outputs, the outputs of RNNs can be dependent on prior elements with the input sequence.

### Long Short-Term Memory (LSTM)

In an example implementation, a Long Short-term Memory is implemented. LSTM are a class of RNNs designed to overcome vanishing and exploding gradients. In RNNs, long term dependencies become more difficult to capture because the parameters or weights either do not change with training or fluctuate rapidly. This occurs when the RNN gradient exponentially decreases to zero, resulting in no change to the weights or parameters, or exponentially increases to infinity, resulting in large changes in the weights or parameters. This exponential effect is dependent on the number of layers and multiplicative gradient. LSTM overcomes the vanishing/exploding gradients by implementing “cells” within the hidden layers of the NN. The “cells” comprise three gates: an input gate, an output gate, and a forget gate. The input gate reduces error by controlling relevant inputs to update the current cell state. The output gate reduces error by controlling relevant memory content in the present hidden state. The forget gate reduces error by controlling whether prior cell states are put in “memory” or forgotten. The gates use activation functions to determine whether the data can pass through the gates. While one skilled in the art would recognize the use of any relevant activation function, example activation functions are sigmoid, tanh, and RELU.

### Convolutional Neural Network (CNN)

In an example implementation, a convolutional neural network is implemented. CNNs is a class of NNs further attempting to replicate the biological neural networks, but of the animal visual cortex. CNNs process data with a grid pattern to learn spatial hierarchies of features. A typical CNN comprises of three layers: convolution, pooling, and fully connected. The convolution and pooling layers extract features, such as those described herein. The convolutional layer comprises of multiple mathematical operations such as of linear operations, a specialized type being a convolution. The fully connected layer combines the extracted features into an output. The input data, such as waveform data, can be represented in a grid, i.e., an array of numbers. A grid of parameters, called a kernel, operates as an optimizable feature extractor and is applied to each position in the grid. Extracted features can become hierarchically more complex as one layer feeds its output into the next layer.

### Matrix Factorization

In an embodiment, Matrix Factorization is implemented. Matrix factorization machine learning exploits inherent relationships between two entities drawn out when multiplied together. Generally, the input features are mapped to a matrix F which is multiplied with a matrix R containing the relationship between the features and a predicted outcome. The resulting dot product provides the prediction. The matrix R is constructed by assigning random values throughout the matrix. In this example, two training matrices are assembled. The first matrix X contains training input features, and the second matrix Z contains the known output of the training input features. First the dot product of R and X are computed and the square mean error, as one example method, of the result is estimated. The values in R are modulated and the process is repeated in a gradient descent style approach until the error is appropriately minimized. The trained matrix R is then used in the machine learning model. In an embodiment, waveform data are used to train the relationship matrix R in a matrix factorization machine learning module. After training, the relationship matrix R and input matrix F, which comprises vector representations of waveform data, results in the prediction matrix P comprising reconstructed waveform data.

### Hidden Markov Model (HMM)

In an embodiment, a hidden Markov model is implemented. An HMM takes advantage of the statistical Markov model to predict an outcome. A Markov model assumes a Markov process, wherein the probability of an outcome is solely dependent on the previous event. In the case of HMM, it is assumed an unknown or “hidden” state is dependent on some observable event. An HMM comprises a network of connected nodes. Traversing the network is dependent on three model parameters: start probability; state transition probabilities; and observation probability. The start probability is a variable that governs, from the input node, the most plausible consecutive state. From there each node i has a state transition probability to node j. Typically the state transition probabilities are stored in a matrix Mᵢⱼ wherein the sum of the rows, representing the probability of state i transitioning to state j, equals 1. The observation probability is a variable containing the probability of output o occurring. These too are typically stored in a matrix Nₒⱼ wherein the probability of output o is dependent on state j. To build the model parameters and train the HMM, the state and output probabilities are computed. This can be accomplished with, for example, an inductive algorithm. Next, the state sequences are ranked on probability, which can be accomplished, for example, with the Viterbi algorithm. Finally, the model parameters are modulated to maximize the probability of a certain sequence of observations. This is typically accomplished with an iterative process wherein the neighborhood of states is explored, the probabilities of the state sequences are measured, and model parameters updated to increase the probabilities of the state sequences. In an embodiment, waveform data are used to train the nodes/states of the HMM machine learning module, which, after training, is used to estimate reconstructed waveform data.

### Support Vector Machine (SVM)

In an embodiment, support vector machines are implemented. SVMs separate data into classes defined by n-dimensional hyperplanes (n-hyperplane) and are used in both regression and classification problems. Hyperplanes are decision boundaries developed during the training process of an SVM. The dimensionality of a hyperplane depends on the number of input features. For example, a SVM with two input features will have a linear (1-dimensional) hyperplane while a SVM with three input features will have a planar (2-dimensional) hyperplane. A hyperplane is optimized to have the largest margin or spatial distance from the nearest data point for each data type. In the case of simple linear regression and classification, a linear equation is used to develop the hyperplane. However, when the features are more complex, a kernel is used to describe the hyperplane. A kernel is a function that transforms the input features into higher dimensional space. Kernel functions can be linear, polynomial, a radial distribution function (or gaussian radial distribution function), or sigmoidal. In an embodiment, waveform data are used to train the linear equation or kernel function of the SVM machine learning module, which, after training, is used to estimate reconstructed waveform data.

### K-Means Clustering (KMC)

In one example embodiment, K-means clustering is implemented. KMC assumes data points have implicit shared characteristics and “clusters” data within a centroid or “mean” of the clustered data points. During training, KMC adds a number of k centroids and optimizes its position around clusters. This process is iterative, where each centroid, initially positioned at random, is re-positioned towards the average point of a cluster. This process concludes when the centroids have reached an optimal position within a cluster. Training of a KMC module is typically unsupervised. In an embodiment, waveform data are used to train the centroids of a KMC machine learning module, which, after training, is used to estimate reconstructed waveform data.

### K-Nearest Neighbor (KNN)

In one example embodiment, K-nearest neighbor is implemented. On a general level, KNN shares similar characteristics to KMC. For example, KNN assumes data points near each other share similar characteristics and computes the distance between data points to identify those similar characteristics but instead of k centroids, KNN uses k number of neighbors. The k in KNN represents how many neighbors will assign a data point to a class, for classification, or object property value, for regression. Selection of an appropriate number of k is integral to the accuracy of KNN. For example, a large k can reduce random error associated with variance in the data but increase error by ignoring small but significant differences in the data. Therefore, a careful choice of k is selected to balance overfitting and underfitting. Concluding whether some data point belongs to some class or property value k, the distance between neighbors is computed. Common methods to compute this distance are Euclidean, Manhattan or Hamming to name a few. In some embodiments, neighbors are given weights depending on the neighbor distance to scale the similarity between neighbors to reduce the error of edge neighbors of one class “out-voting” near neighbors of another class. In one example embodiment, k is 1 and a Markov model approach is utilized. In an embodiment, waveform data are used to train a KNN machine learning module, which, after training, is used to estimate reconstructed waveform data.

To perform one or more of its functionalities, the machine learning module can communicate with one or more other systems. For example, an integration system can integrate the machine learning module with one or more email servers, web servers, one or more databases, or other servers, systems, or repositories. In addition, one or more functionalities can utilize communication between a user and the machine learning module.

Any one or more of the modules described herein can be implemented using hardware (e.g., one or more processors of a computer/machine) or a combination of hardware and software. For example, any module described herein can configure a hardware processor (e.g., among one or more hardware processors of a machine) to perform the operations described herein for that module. In some example implementations, any one or more of the modules described herein can comprise one or more hardware processors and can be configured to perform the operations described herein. In an implementation, one or more hardware processors are configured to include any one or more of the modules described herein.

Moreover, any two or more of these modules can be combined into a single module, and the functions described herein for a single module can be subdivided among multiple modules. Furthermore, according to various example implementations, modules described herein as being implemented within a single machine, database, or device can be distributed across multiple machines, databases, or devices. The multiple machines, databases, or devices are communicatively coupled to enable communications between the multiple machines, databases, or devices. The modules themselves are communicatively coupled (e.g., via appropriate interfaces) to each other and to various data sources, to allow information to be passed between the applications so as to allow the applications to share and access common data.

Further implementations are illustrated in the following Examples which are given for illustrative purposes only and are not intended to limit the scope of the described techniques.

### Example Application

### I—Unsupervised Deep Learning of Electrocardiograms Enables Scalable Human Disease Profiling

The 12-lead electrocardiogram (ECG) is inexpensive and widely available. A deep learning denoising autoencoder was developed and associations between ECG encodings and approximately 1,600 Phecode-based diseases were systematically evaluated in three datasets separate from model development, and the results were meta-analyzed. The latent space ECG model identified associations with 645 prevalent and 606 incident Phecodes. Associations were most enriched in the circulatory (n=140, 82% of category-specific Phecodes), respiratory (n=53, 62%) and endocrine/metabolic (n=73, 45%) categories, with additional associations across the phenome. The strongest ECG association was with hypertension (p<2.2×10⁻³⁰⁸). The ECG latent space model demonstrated more associations than models using standard ECG intervals, and offered favorable discrimination of prevalent disease compared to models comprising age, sex, and race. The latent space models were further used to generate disease-specific ECG waveforms and facilitate individual disease profiling.

The modern resting electrocardiogram (ECG) utilizes waveform data generated from surface electrodes to represent cardiac activation and impulse conduction. Introduced in the early 1900s, the original ECG was primarily used for arrhythmia detection, but its diagnostic utility expanded rapidly to include the identification of coronary artery disease and other cardiac structural abnormalities. It has now become clear that non-cardiac diseases, from electrolyte derangements to central nervous system pathology, also cause characteristic changes in the ECG waveform.

Recent advances in machine learning have revealed that the ECG contains diagnostic and prognostic information that extends beyond traditional clinical interpretation. Low-dimensional representations of ECGs constructed from deep learning models can detect cardiac diseases such as left ventricular dysfunction and paroxysmal atrial fibrillation for patients in sinus rhythm. Other models have demonstrated predictive power beyond the cardiovascular system, estimating factors such as age, sex, serum potassium, and one-year mortality with high accuracy.

Modern electronic health record (EHR)-based technology has made available large, detailed datasets with rich phenotypic information, enabling large-scale disease-based association testing. Phenome-wide association studies (PheWAS) facilitate high-throughput association testing between predictor variables and multiple disease states using electronically ascertained diagnostic codes. Diseases are commonly represented by Phecodes, or standardized, aggregated groupings of International Classification of Disease (ICD) codes.

The present Example Application harnessed the inferential capabilities of deep learning models and the analytic power of PheWAS to comprehensively assess the array of disease states that manifest in the ECG waveform. Given the growing number of consumer-based wearable devices capable of recording single-lead ECGs, parallel analyses were performed using both traditional 12-lead ECGs and data derived only from lead I, a common vector used for consumer-based ECG recording. Specifically, deep learning models known as denoising autoencoders were trained to encode 12- and single-lead ECGs within a latent space using a large primary care sample. The autoencoder model was selected because it is unsupervised and optimized to learn ECG waveform features alone, without additional information regarding patient demographics or clinical outcomes. The position of ECG representations in the latent space was then used to perform high-throughput association testing with roughly 1600 prevalent and incident diseases across three independent datasets spanning over 150,000 individuals. The latent space modeling was further used to display characteristic ECG features for detectable conditions and to clinically profile individual patients.

Accordingly, the Example Application combines the representational richness afforded by deep learning with systematic association testing across multiple large datasets to comprehensively characterize the full spectrum of disease states manifest in the ECG waveform. The Example Application demonstrates that an AI-enabled encoding of the ECG associates with a wide array of cardiac and non-cardiac disease, and thereby demonstrates utility as an efficient screening tool for an array of conditions in the ambulatory care setting.

### Results

### Study Sample and Autoencoder Development

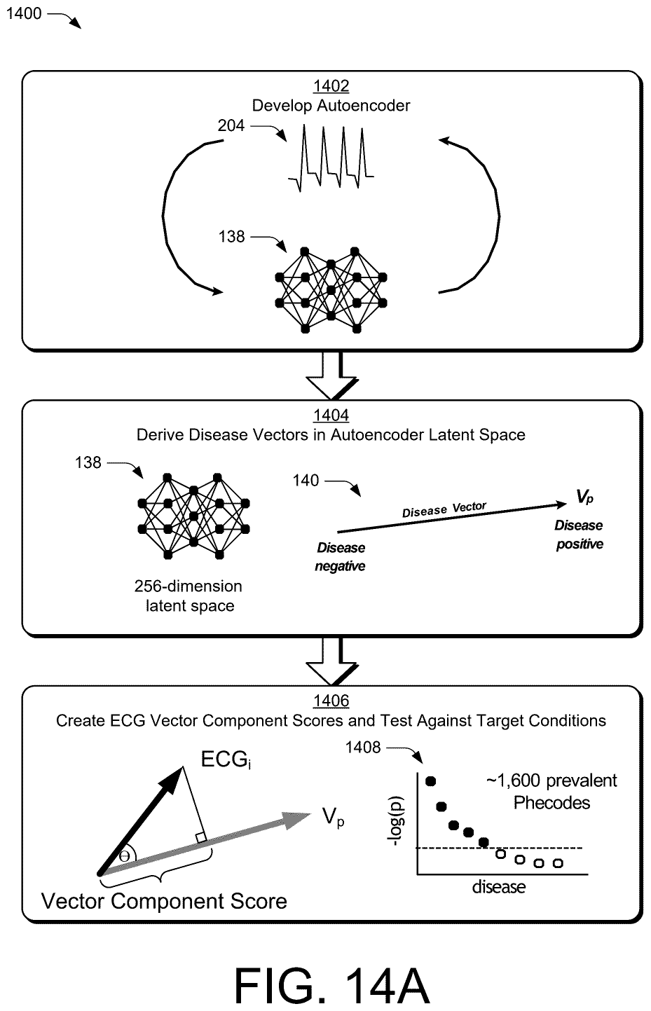

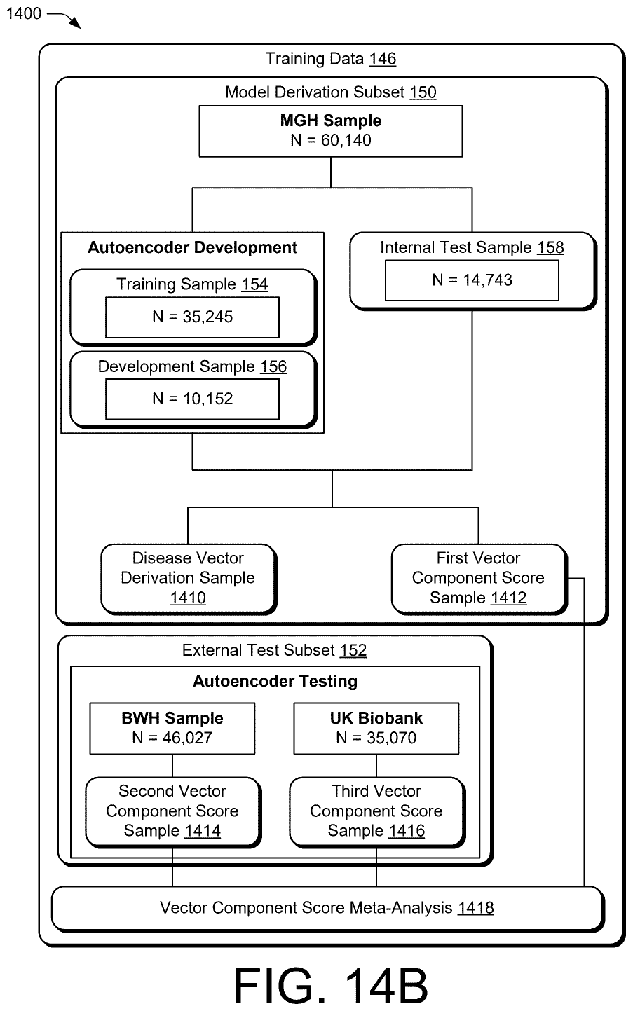

The study utilized three nonoverlapping, independent datasets spanning over 150,000 individuals, each of which contained individual-level demographic and clinical information, including 12-lead ECGs (FIGS. 14A and 14B). Two datasets were taken from the Community Care Cohort Project (C3PO), a previously established cohort comprising adults aged ≥18 years who received longitudinal primary care within the Mass General Brigham network between 2000-2018, which is linked by a common EHR data warehouse. The Massachusetts General Hospital (MGH) C3PO dataset included 60,140 primary care patients and the Brigham and Women's Hospital (BWH) C3PO dataset included 46,027 primary care patients. The third dataset comprised 35,070 participants from the UK Biobank, a prospective national biorepository that enrolled individuals aged 40-60 years between 2006-2010, with deep phenotyping data, baseline questionnaires, and linkage to electronic health record data. Characteristics of participants by dataset are provided in Table 1.

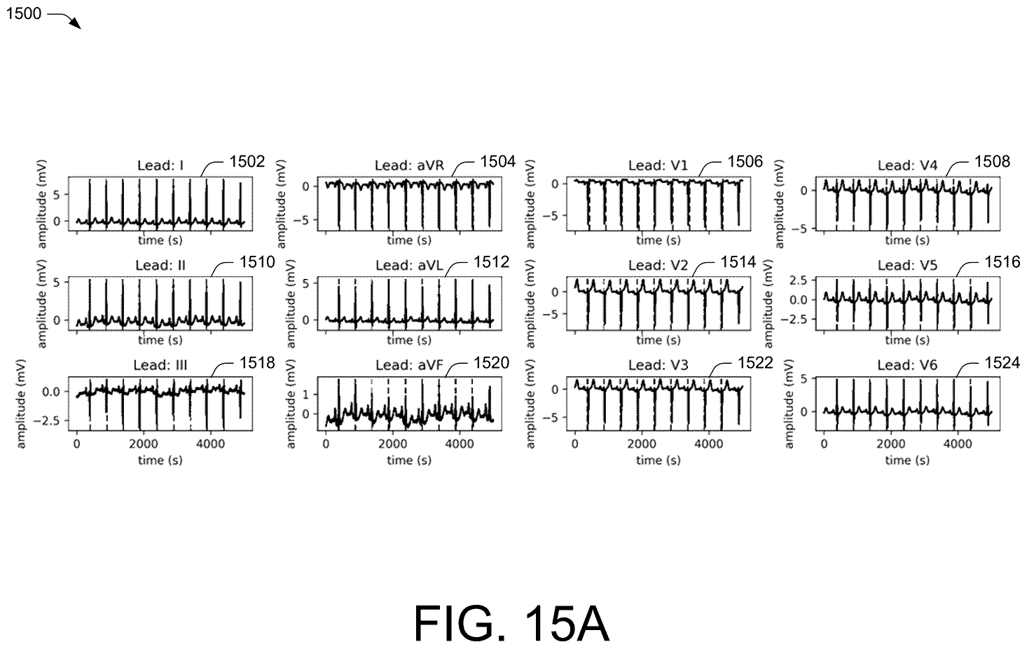

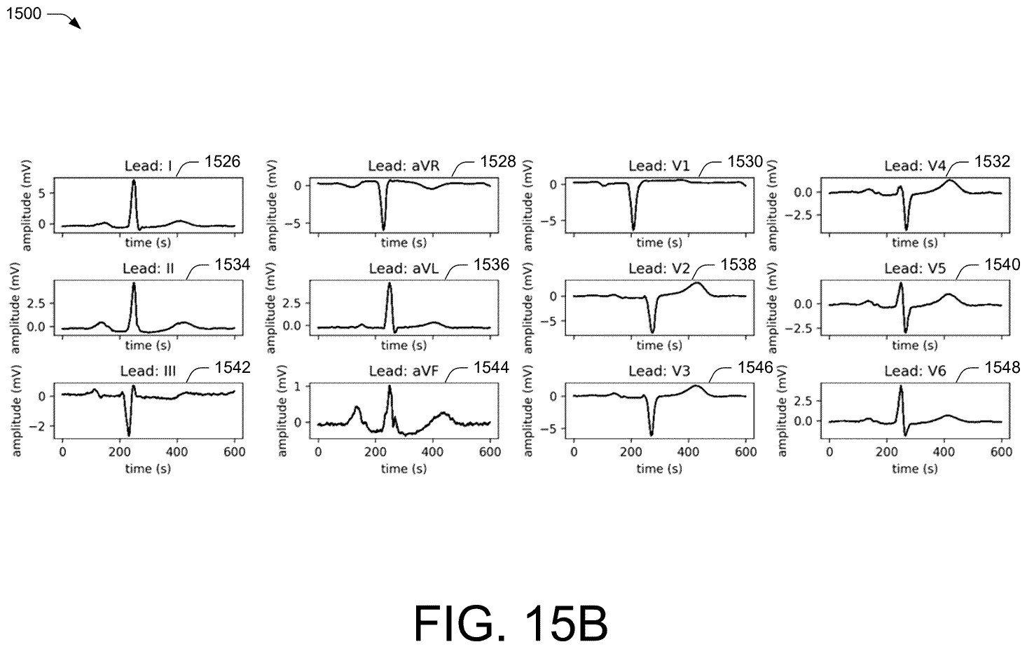

For both 12- and single-lead models, autoencoders were developed using ECGs from the MGH-C3PO dataset, including 35,245 for training, 10,152 for validation, and 6,674 for testing. The best-performing architecture contained over 11 million neurons and used mish activations, 2 dense convolutional blocks (each with 5 layers of convolutions per block), a 71 timestep convolutional kernel, and layer normalization, with 256 neurons in the fully connected layer. Example architectures are described herein with respect to FIGS. 2-6. The models were trained to reconstruct a median waveform corresponding to a single PQRST cycle (FIG. 15B). To assess the accuracy of reconstruction in each of the three datasets, voltages were pooled across test set ECGs and compared to the voltages generated from reconstructions. For each dataset, the average per-voltage Pearson correlation coefficient and the 95% confidence interval based on 1000 bootstrap resamplings were assessed. This method was selected, as opposed to comparing per-person pooled voltages across test set and reconstructed ECGs, to assess the model's global performance in ECG reconstruction rather than its performance reproducing ECGs from specific individuals within the datasets. High-fidelity reconstruction of novel ECGs was observed with Pearson correlation coefficients of 0.9956 (95% CI 0.9931-0.9972) in the MGH-C3PO testing set; 0.9916 (95% CI 0.9853-0.9950) in BWH-C3PO; and 0.9526 (95% CI 0.9427-0.9617) in the UK Biobank.

FIGS. 14A and 14B show an example 1400 of autoencoder and phenotype vector derivation for latent space phenome-wide association studies (PheWAS). FIG. 14A shows a flow diagram of steps performed in developing the autoencoder and the phenotype vector derivation for the PheWAS studies, and FIG. 14B provides a breakdown of datasets used.

In the Example Application, the flow diagram was conducted in parallel for both 12-lead and single-lead electrocardiogram (ECG) models. FIG. 14A shows a block 1402 where the autoencoder was developed, a block 1404 where disease vectors in autoencoder latent space were derived, and a block 1406 where ECG vector component scores were created and tested against target conditions.

The block 1402 illustrates how the autoencoder (e.g., the deep learning autoencoder 134) was developed using the ECG input 204. The autoencoder processed the ECG input 204 to generate encodings within the latent space 138. The block 1404 illustrates how the at least one disease vector 140 was derived within the latent space 138 (e.g., a 256-dimension latent space). By way of example, the example 1400 generated the at least one disease vector 140 by analyzing the distribution of ECG encodings between disease-positive and disease-negative cases in the latent space 138. The block 1406 illustrates how the vector component scores were calculated by projecting individual ECG encodings onto the disease vectors. A plot 1408 shows the distribution of test statistics for approximately 1,600 prevalent disease conditions, with filled circles representing significant associations and open circles representing non-significant associations.

As outlined in FIG. 14B, the autoencoder was trained to encode and reconstruct 12- and single-lead ECGs using the Massachusetts General Hospital (MGH) subset of the Community Care Cohort Project (C3PO) dataset (MGH-C3PO) (e.g., the training sample 154). The autoencoder was tested in three test sets without modification: a) an MGH-C3PO holdout set (e.g., the internal test sample 158), b) the independent Brigham and Women's Hospital (BWH) subset of C3PO (BWH-C3PO) (e.g., a first portion of the external test subset 152), and c) the UK Biobank prospective cohort study (e.g., a second portion of the external test subset 152). To assess for associations with disease, phenotype vectors were derived using labeled ECGs from 50% of the MGH-C3PO dataset (e.g., a disease vector derivation sample 1410), and those vectors were projected onto each test set without modification. For every individual in each test set, the projected component, or the position along each phenotype vector (hereafter termed “vector component score”), was calculated, and associations between vector component scores and corresponding Phecodes were tested. For example, a first vector component score sample 1412 was derived from the internal test sample 158, a second vector component score sample 1414 was derived from the BWH sample, and a third vector component score sample 1416 was derived from the UK Biobank sample. Sample-level PheWAS was performed in each of the three datasets and then the results were meta-analyzed, represented as vector component score meta-analysis 1418.

FIGS. 15A and 15B illustrate an example 1500 of ECG waveforms in a 12-lead format. FIG. 15A shows original ECG tracings, while FIG. 15B presents corresponding reconstructed median waveforms. Both figures display waveforms for leads I, II, III, aVR, aVL, aVF, V1, V2, V3, V4, V5, and V6 arranged in a grid. FIG. 15A includes a lead I waveform 1502, a lead aVR waveform 1504, a lead V1 waveform 1506, a lead V4 waveform 1508, a lead II waveform 1510, a lead aVL waveform 1512, a lead V2 waveform 1514, a lead V5 waveform 1516, a lead III waveform 1518, a lead aVF waveform 1520, a lead V3 waveform 1522, and a lead V6 waveform 1524. FIG. 15B includes a lead I waveform 1526, a lead aVR waveform 1528, a lead V1 waveform 1530, a lead V4 waveform 1532, a lead II waveform 1534, a lead aVL waveform 1536, a lead V2 waveform 1538, a lead V5 waveform 1540, a lead III waveform 1542, a lead aVF waveform 1544, a lead V3 waveform 1546, and a lead V6 waveform 1548.

The reconstructed waveforms in FIG. 15B represent the output of the autoencoder model, demonstrating its ability to capture and reproduce features of the original ECG signals shown in FIG. 15A. That is, the lead I waveform 1526 corresponds to the lead I waveform 1502, the lead a VR waveform 1528 corresponds to the lead a VR waveform 1504, the lead V1 waveform 1530 corresponds to the lead V1 waveform 1506, the lead V4 waveform 1532 corresponds to the lead V4 waveform 1508, the lead II waveform 1534 corresponds to the lead II waveform 1510, the lead aVL waveform 1536 corresponds to the lead aVL waveform 1512, the lead V2 waveform 1538 corresponds to the lead V2 waveform 1514, the lead V5 waveform 1540 corresponds to the lead V5 waveform 1516, the lead III waveform 1542 corresponds to the lead III waveform 1518, the lead aVF waveform 1544 corresponds to the lead aVF waveform 1520, the lead V3 waveform 1546 corresponds to the lead V3 waveform 1522, and the lead V6 waveform 1548 corresponds to the lead V6 waveform 1524.

### Phenotype Vector Derivation and ECG Projection

For phenotype vector derivation, 50% of ECGs from the MGH-C3PO dataset were mapped to a library of 1,866 Phecodes, grouped across 17 disease categories (e.g., circulatory system, endocrine/metabolic, genitourinary).

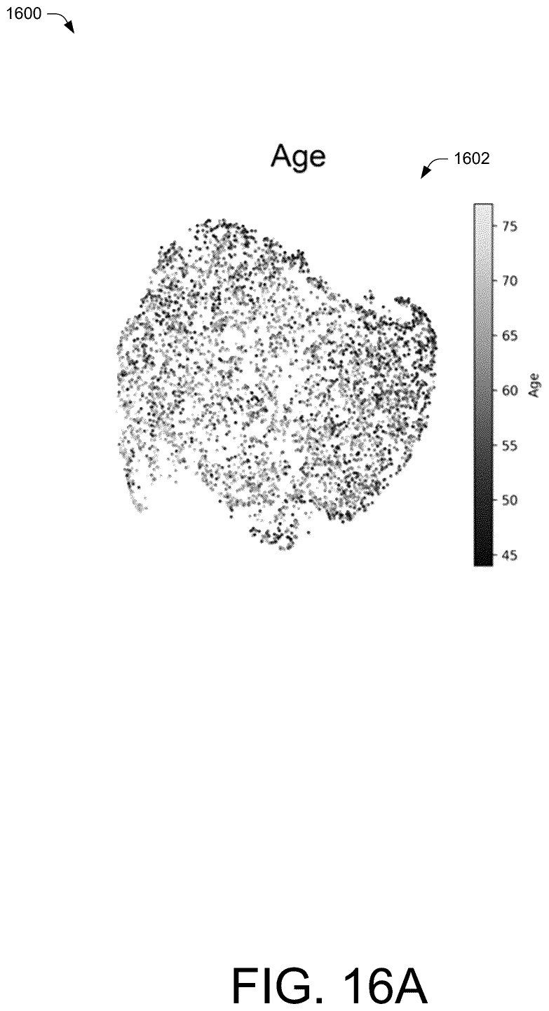

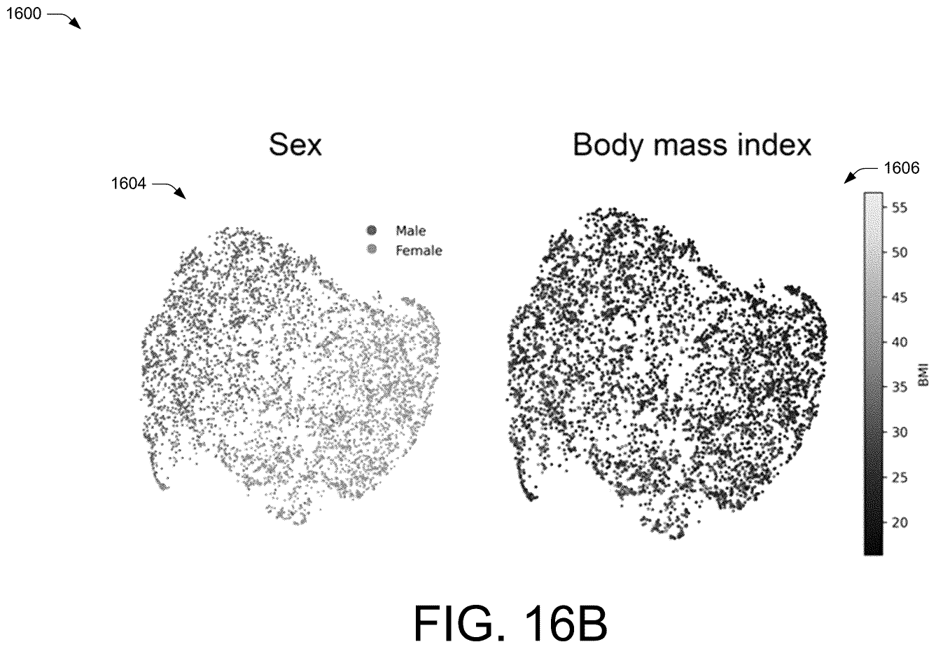

Labeled ECG encodings were used to derive phenotype vectors within the latent space. The remaining unlabeled ECGs across the three datasets (n=30,070 for MGH-C3PO; n=46,027 for BWH-C3PO; and n=35,070 for UK Biobank) were then encoded and the position of each ECG along each phenotype vector was determined to generate disease-specific vector component scores. Scores were scalar values ranging [−10.1, 8.5] in MGH, [−10.3, 8.3] in BWH, and [−7.8, 5.6] in the UK Biobank. Representations of age, sex, and body mass index in the latent space are shown in FIGS. 16A and 16B, and a schematic summarizing the phenotype vector concept is displayed in FIG. 9.

FIGS. 16A and 16B illustrate an example 1600 of uniform manifold approximation and projections (UMAPs) depicting the ECG latent space. FIG. 16A shows a first UMAP 1602 stratified by age. FIG. 16B presents a second UMAP 1604 stratified by sex and a third UMAP 1606 stratified by body mass index. The UMAPs were derived using ECG encodings in latent space and visually represent the distribution of different patient characteristics within the high-dimensional ECG latent space, demonstrating how the autoencoder model captures and organizes these demographic and physical attributes from the ECG waveform data alone.

### Latent Space PheWAS

A PheWAS was then performed in the remaining MGH-C3PO samples. To assess the validity of results prior to downstream analysis, empiric perturbation testing was performed by randomly reclassifying samples based on Phecode presence or absence. Quantile-quantile plots were generated for MGH-C3PO 12-lead results as well as random Phecode reclassification levels of 10%, 20%, and 100%. As expected, the number of significant associations decreases as the degree of random reclassification increases.

The PheWAS was then performed in each of the independent validation sets. After filtering out Phecodes with less than 100 combined cases, those present in only one dataset, and/or those for which model convergence failed, the study-specific results for 1595 Phecodes for the 12-lead model and 1,600 Phecodes for the single-lead model were meta-analyzed. Using a Bonferroni-corrected two-sided p-value of 3.1×10⁻⁵ (0.05/1595 and 0.05/1600), significant associations between latent space position and disease status were observed for 643 Phecodes in the 12-lead model (40% of overall Phecodes) and 565 Phecodes in the single-lead model (35%), respectively. The circulatory system category comprised the greatest enrichment of significant associations (n=140, or 82% of Phecodes in this category for the 12-lead model, and n=139, or 81% for the single-lead model). Enrichment was also observed within the respiratory (n=53, 62% of category-specific Phecodes for the 12-lead model; n=46, 54% for the single-lead model) and endocrine/metabolic categories (n=73, 45% of category-specific Phecodes for the 12-lead model; n=72, 44% for the single-lead model).

For the 12-lead model, the latent space positions for the Phecodes for hypertension, including “hypertension” (odds ratio [OR] per 1-point increase in vector component score 1.24, 95% CI 1.23-1.26, p<2.2×10⁻³⁰⁸) and “essential hypertension” (OR 1.24, 95% CI 1.23-1.26, p<2.2×10⁻³⁰⁸) showed the strongest associations (i.e., smallest p-values), followed by “cardiomyopathy” (OR 1.75, 95% CI 1.71-1.79, p<2.2×10⁻³⁰⁸). The strongest associations with non-cardiac Phecodes included “obesity” (OR 1.30, 95% CI 1.29-1.32, p<2.2×10⁻³⁰⁸), “diabetes mellitus” (OR 1.26, 95% CI 1.24-1.27, p=5.9×10⁻³⁰⁴), “disorders of fluid, electrolyte, and acid-base balance” (OR 1.28, 95% CI 1.26-1.30, p=5.5×10⁻²⁵⁸), and “pulmonary congestion and hypostasis” (OR 1.59, 95% CI 1.55-1.63, p=1.2×10⁻²⁴⁵). For the single-lead model, the latent space positions for the Phecodes for cardiomyopathy, including “cardiomyopathy” (OR 1.22, 95% CI 1.21-1.23, p<2.2×10⁻³⁰⁸) and “primary/intrinsic cardiomyopathies” (OR 1.24, 95% CI 1.23-1.26, p<2.2×10⁻³⁰⁸) showed the strongest associations, followed by “congestive heart failure NOS” (OR 1.22, 95% CI 1.21-1.23, p<2.2×10⁻³⁰⁸).

When ranked by effect estimate, associations with substantial effect sizes were observed across all disease categories, but in particular for cardiac Phecodes (e.g., “Cardiac defibrillator in situ” OR 2.18, 95% CI 2.04-2.32, p=6.83×10⁻¹²⁴) (Table 2). Similar patterns were observed using the single-lead model. Additionally, unexpected and highly robust relationships were identified, including tobacco use disorder (12-lead: OR 1.19, 95% CI 1.18-1.21, p=1.0×10⁻¹⁴⁹; single-lead: OR 1.08, 95% CI 1.08-1.09, p=1.9×10⁻¹³¹), fever of unknown origin (OR 1.18, 95% CI 1.16-1.19, p=5.5×10⁻¹³⁶; OR 1.07, 95% CI 1.07-1.08, p=1.2×10⁻¹⁰⁴), and non-alcoholic liver disease (OR 1.17, 95% CI 1.15-1.20, p=8.0×10⁻⁵¹; OR 1.05, 95% CI 1.05-1.06, p=1.9×10⁻³⁹). Effects were generally consistent across datasets, including in the UK Biobank, which had an overall lower prevalence of disease. Top associations by effect size across disease category for incident disease with respect to hazard ratios are shown in Table 3.

### ECG Intervals PheWAS

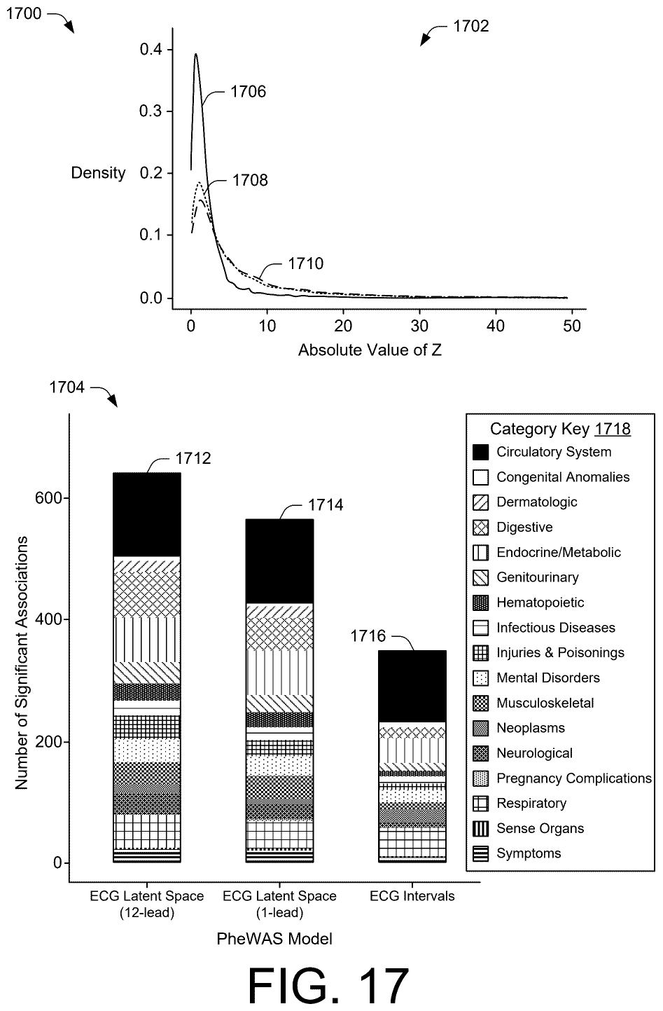

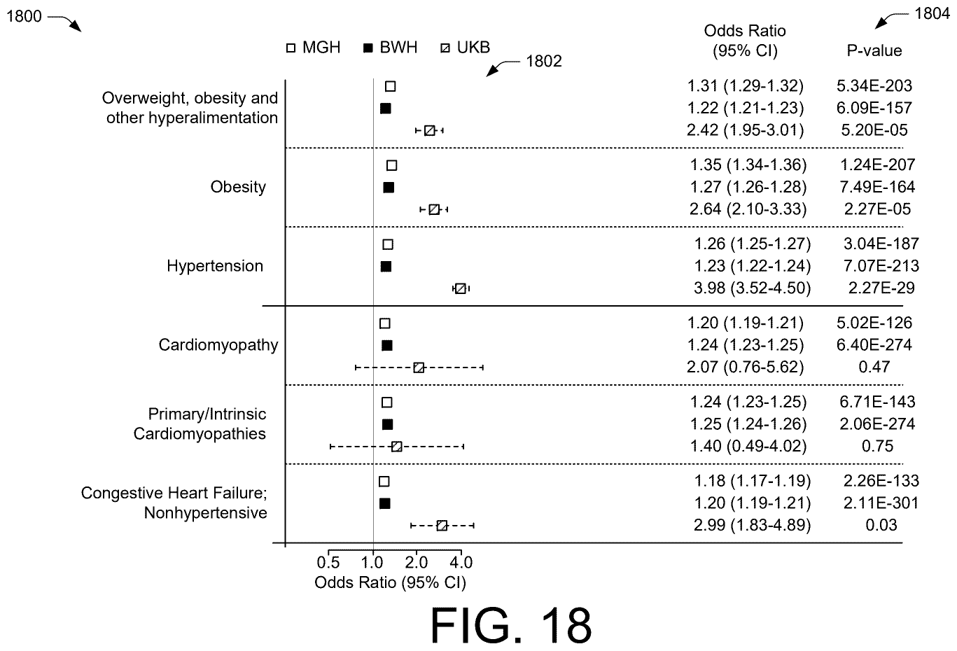

Meta-analyses of the ECG intervals PheWAS included 1607 Phecodes for the PR interval; 1607 Phecodes for the QRS duration; and 1605 Phecodes for the QT interval. In comparing the ECG intervals and latent space models, the analysis was restricted to Phecodes that were present in all meta-analyses (n=1584). Using the smallest meta-analyzed p-value across any ECG interval for each Phecode, fewer significant associations were observed for the intervals models relative to both the 12-lead and single-lead latent space models, both overall and within disease categories (FIG. 17). Forest plots summarizing the associations for the top Phecodes are displayed in FIG. 18.

FIG. 17 illustrates an example 1700 comparing test statistic distributions and significant associations across different electrocardiogram analysis models. The example 1700 includes a test statistic distribution plot 1702 and a significant associations graph 1704. The test statistic distribution plot 1702 displays the absolute value of Z scores on the x-axis and density on the y-axis for three different models and includes an ECG interval model plot 1706 (solid line), a 12-lead ECG latent space model plot 1708 (smaller-dashed line), and a 1-lead ECG latent space model plot 1710 (larger-dashed line). The significant associations graph 1704 shows the number of significant associations across different disease categories for three models: a 12-lead ECG latent space model 1712, a 1-lead ECG latent space model 1714, and an ECG intervals model 1716. Significant associations are defined as those exceeding a Bonferroni-corrected two-sided p-value of 3.1×10⁻⁵. For the ECG intervals model 1716, a result is considered significant if the meta-analyzed p-value for any of the tested ECG intervals (PR, QRS, QT) exceeds the significance threshold. A category key 1718 identifies different disease categories. The significant associations graph 1704 demonstrates that the latent space models (e.g., the 12-lead ECG latent space model 1712 and the 1-lead ECG latent space model 1714) yield a greater number of significant associations compared to the ECG intervals model 1716, both overall and across various disease categories.

FIG. 18 illustrates an analysis 1800 summarizing the top Phecode associations using 12-lead ECG and single-lead ECG models in the meta-analysis. The analysis 1800 includes a forest plot 1802 and statistics 1804. The forest plot 1802 displays odds ratios and confidence intervals for different Phecodes, with separate sections for 12-lead ECG (top three Phecodes) and single-lead ECG (bottom three Phecodes) results. Each row in the forest plot 1802 represents a specific Phecode association, with points depicting study-specific odds ratios per 1-point increase in vector component score. The forest plot 1802 uses different markers to distinguish between datasets: white- or open-filled squares represent data from the MGH dataset, black-filled squares represent data from the BWH dataset, and diagonally-filled squares represent data from the UK Biobank (UKB) dataset. Horizontal lines extending from these datapoints represent 95% confidence intervals for each odds ratio. The statistics 1804 section provides numerical odds ratio (OR) values with 95% confidence intervals (CI) and p-values for each Phecode association shown in the forest plot 1802.

### Discrimination of Phecode Diseases

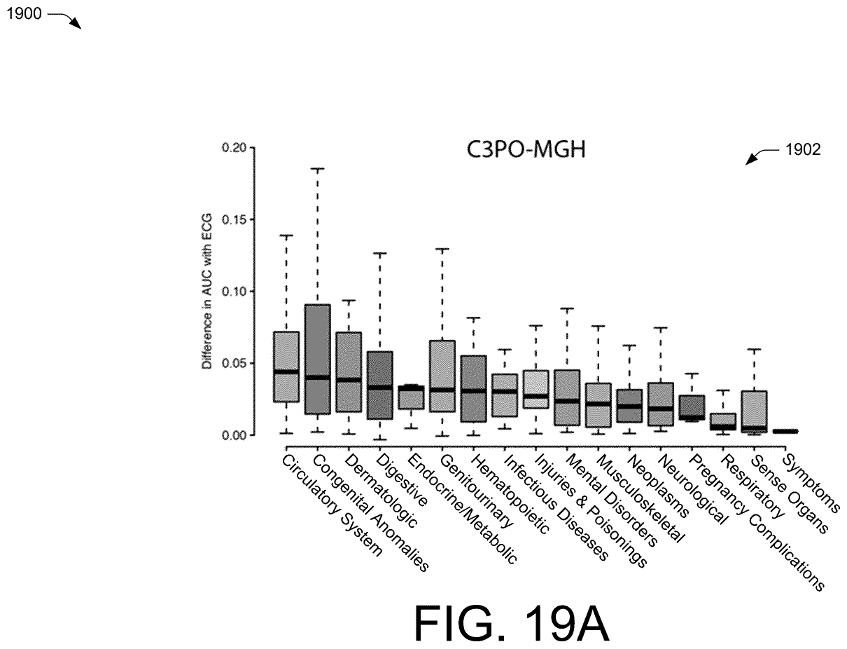

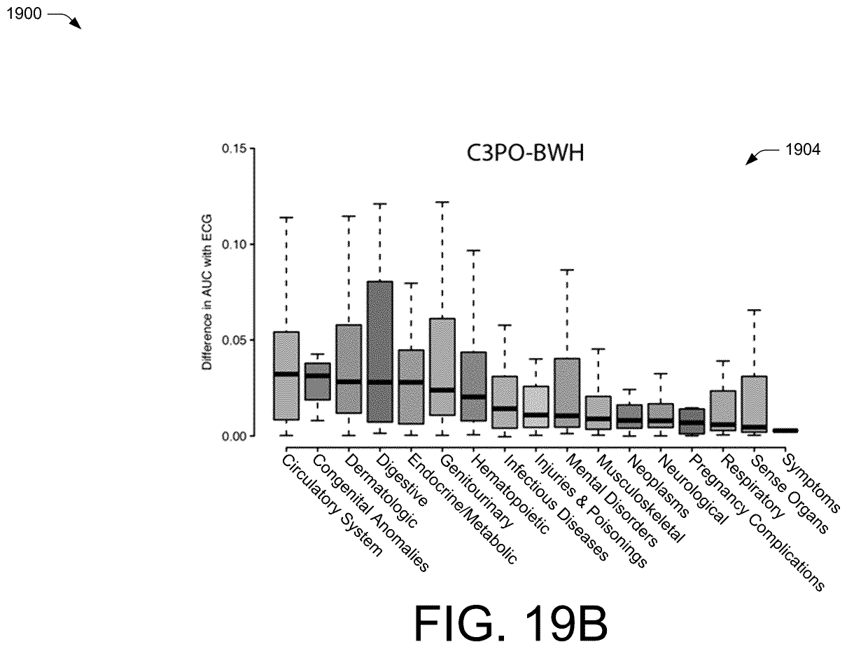

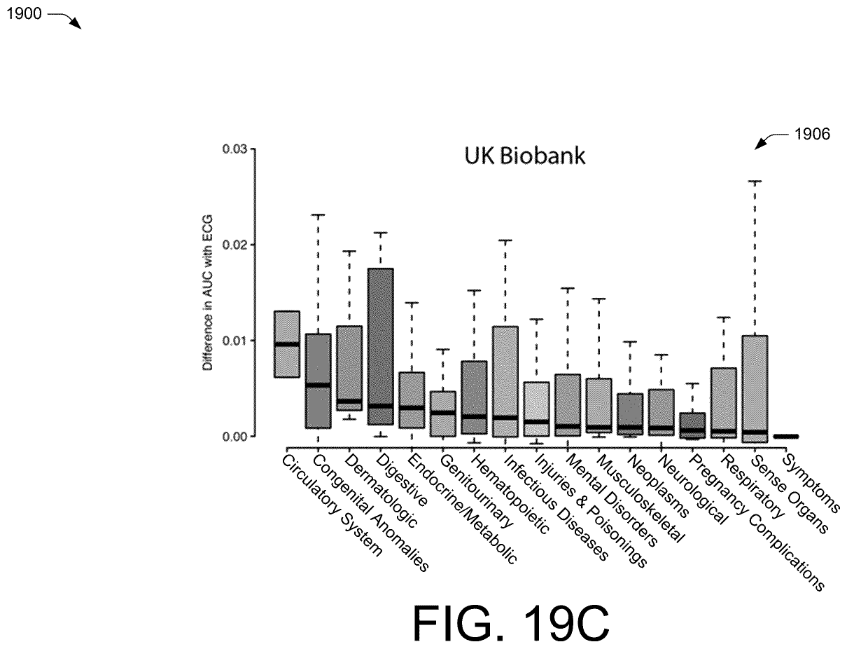

Discrimination was then compared using the area under the receiver operator characteristic curve (AUC) across logistic regression models in which Phecodes having significant associations with the ECG in the primary meta-analysis were regressed on age, sex, and race, with or without an additional term for ECG vector component scores. Substantial increases in discrimination were generally observed for models that included the ECG vector component score term as compared to models that did not, particularly for circulatory system (median difference in AUC, interquartile range for MGH-C3PO 0.031, 0.016-0.065; BWH-C3PO 0.024, 0.010-0.06; UKB 0.0053, 0.00086-0.011) and respiratory (MGH-C3PO 0.044, 0.023-0.072; BWH-C3PO 0.028, 0.011-0.058; UKB 0.0015, 0.0019-0.0056) Phecode categories, though additional improvements in discrimination were observed for other Phecode categories as well (FIGS. 19A-19C). Substantial AUC improvement persisted when the ECG latent space was compared to models including standard ECG intervals.

FIGS. 19A-19C illustrate an analysis 1900 summarizing the difference in the area under the receiver operator characteristic curve (AUC) between models with and without ECG vector component scores, stratified by Phecode category and dataset. FIG. 19A shows a C3PO-MGH plot 1902, FIG. 19B shows a C3PO-BWH plot 1904, and FIG. 19C shows a UK Biobank plot 1906. Only conditions meeting Bonferroni-corrected phenome-wide significance in the primary meta-analysis (p<3.1×10⁻⁵) are included. Multivariable logistic regression models were fit with adjustment for age, sex, race, and vector component score. In the MGH-C3PO plot 1902 and the BWH-C3PO plot 1904, models were further adjusted for ECG acquisition date and zero padding.

### Latent Space Incident Disease PheWAS

In an exploratory analysis using the 12-lead ECG model, significant associations were observed between the vector component scores and incident disease for 457 out of 1370 tested Phecodes (33.4%). Similar to the analyses focused on existing conditions, the greatest enrichment for associations was observed among circulatory (n=107, 74% of Phecodes in this category), endocrine/metabolic (n=60, 52% of category-specific Phecodes) and digestive (n=53, 38% of category-specific Phecodes) conditions. Significant associations included considerable effect sizes for a variety of conditions including incident paroxysmal ventricular tachycardia (hazard ratio [HR] per 1-point increase in vector component score 1.61, 95% CI 1.53-1.70, p=2.58×10⁻⁶⁵), end stage renal disease (HR 1.31, 95% CI 1.23-1.40, p=8.49×10⁻¹⁸), and respiratory failure (HR 1.35, 95% CI 1.30-1.40, p=2.16×10⁻⁵²).

### Model-Based, Disease-Specific Median Waveforms

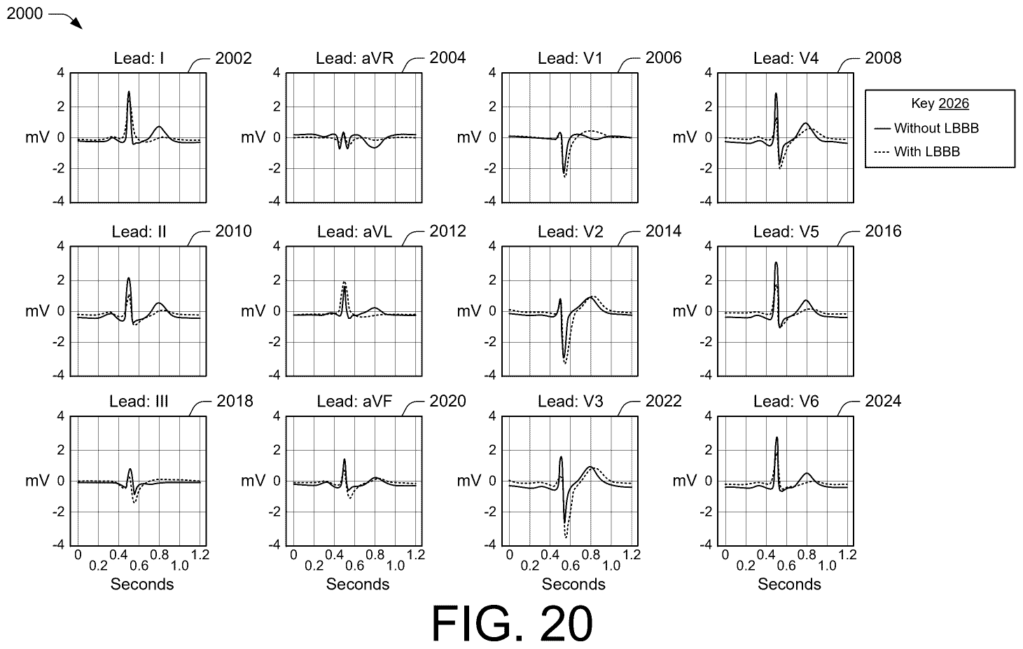

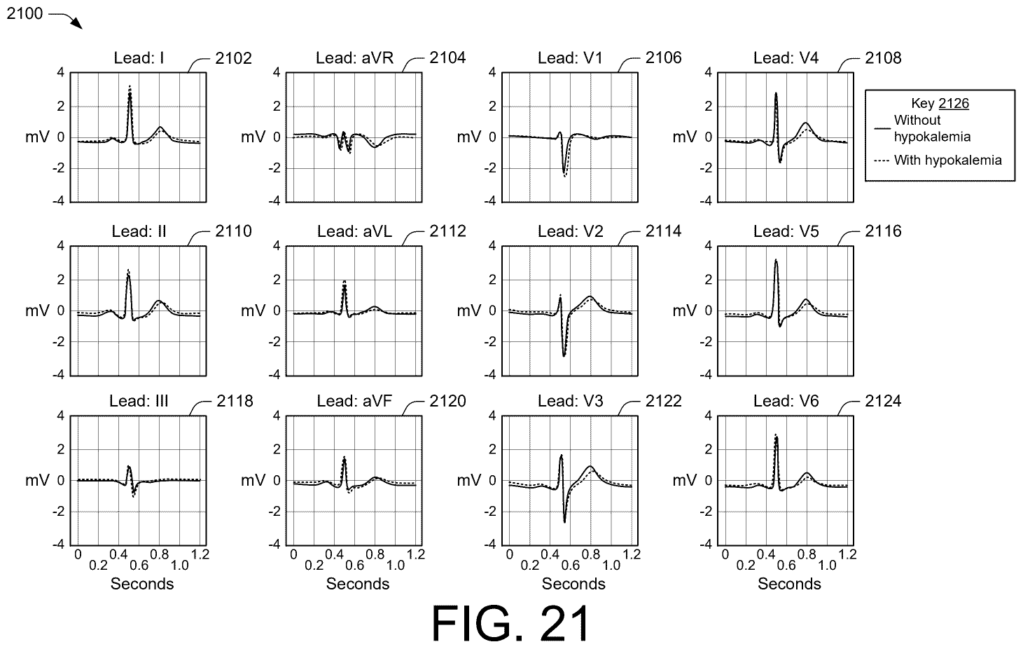

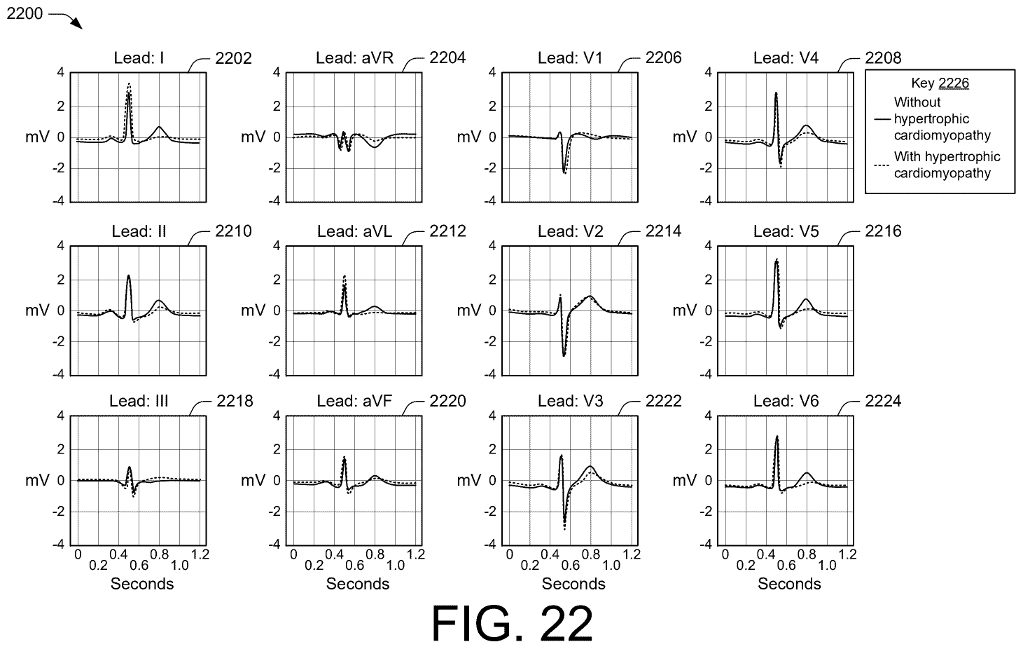

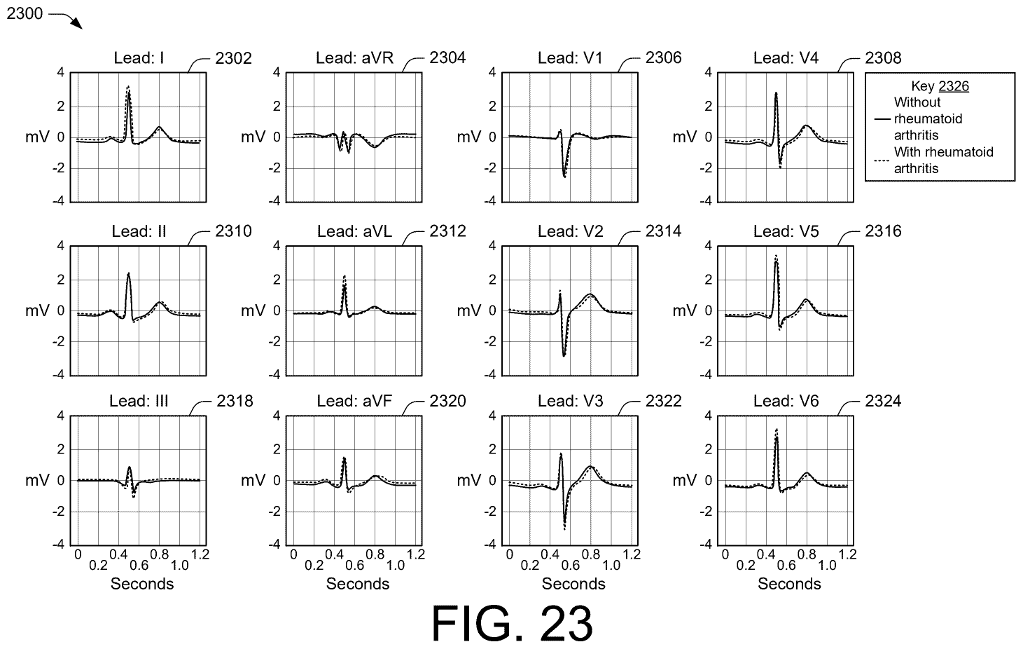

For certain diseases with well-characterized ECG manifestations, model-derived features were consistent with expectations. For example, median waveforms generated from the left bundle branch block disease-positive centroid demonstrated QRS widening, smaller initial r waves in the right-sided precordial leads (V1-V3), and R wave slurring in the left-sided leads (I, aVL, V5, V6) relative to median waveforms generated from the disease-negative centroid (FIG. 20). For hypokalemia, model-derived features included decreased T wave amplitude and relative QT prolongation (FIG. 21). In other instances, however, reconstructed disease case and control ECGs appeared morphologically similar visually despite highly significant differences in projected components from the latent space models. For example, model-derived ECG reconstructions of hypertrophic cardiomyopathy were notable for broad, flattened T-waves, particularly in the left-sided leads, which are more subtle than the classical well-defined ECG manifestations of hypertrophic cardiomyopathy (e.g., prominent precordial voltages, repolarization abnormalities/T-wave inversions, pathologic Q waves) (FIG. 22). Likewise, rheumatoid arthritis, an inflammatory condition associated with higher risk of cardiovascular disease which had a strong ECG vector component score association but has no clinically characteristic ECG signature, was notable for subtle differences in T wave morphology and QT interval (FIG. 23). Overall findings suggest that the latent space is sensitive to subtle manifestations of disease, while the model-derived ECG reconstructions are conservative and may not visually replicate all hallmarks of disease.

FIG. 20 illustrates an example 2000 of median waveform reconstructions for centroids reflecting individuals without and with left bundle branch block (LBBB). A 12-lead electrocardiogram is shown, including a lead I waveform plot 2002, a lead aVR waveform plot 2004, a lead V1 waveform plot 2006, a lead V4 waveform plot 2008, a lead II waveform plot 2010, a lead aVL waveform plot 2012, a lead V2 waveform plot 2014, a lead V5 waveform plot 2016, a lead III waveform plot 2018, a lead aVF waveform plot 2020, a lead V3 waveform plot 2022, and a lead V6 waveform plot 2024. Each lead shows two waveforms: a solid line representing individuals without LBBB and a dashed line representing individuals with LBBB. A key 2026 indicates the line styles used to differentiate between individuals without LBBB (solid line) and those with LBBB (dashed line). Each waveform plot displays voltage (in millivolts, mV) on the vertical axis and time on the horizontal axis (in seconds). The waveforms demonstrate characteristic differences in ECG patterns between individuals with and without LBBB across all leads.

FIG. 21 illustrates an example 2100 of median waveform reconstructions for centroids reflecting individuals without and with hypokalemia. A 12-lead electrocardiogram is shown, including a lead I waveform plot 2102, a lead aVR waveform plot 2104, a lead V1 waveform plot 2106, a lead V4 waveform plot 2108, a lead II waveform plot 2110, a lead aVL waveform plot 2112, a lead V2 waveform plot 2114, a lead V5 waveform plot 2116, a lead III waveform plot 2118, a lead aVF waveform plot 2120, a lead V3 waveform plot 2122, and a lead V6 waveform plot 2124. Each lead shows two waveforms: a solid line representing individuals without hypokalemia and a dashed line representing individuals with hypokalemia. A key 2126 indicates the line styles used to differentiate between individuals without hypokalemia (solid line) and those with hypokalemia (dashed line). Each waveform plot displays voltage (in millivolts, mV) on the vertical axis and time on the horizontal axis (in seconds). The waveforms demonstrate characteristic differences in ECG patterns between individuals with and without hypokalemia across all leads.

FIG. 22 illustrates an example 2200 of median waveform reconstructions for centroids reflecting individuals without and with hypertrophic cardiomyopathy. A 12-lead electrocardiogram is shown, including a lead I waveform plot 2202, a lead aVR waveform plot 2204, a lead V1 waveform plot 2206, a lead V4 waveform plot 2208, a lead II waveform plot 2210, a lead aVL waveform plot 2212, a lead V2 waveform plot 2214, a lead V5 waveform plot 2216, a lead III waveform plot 2218, a lead aVF waveform plot 2220, a lead V3 waveform plot 2222, and a lead V6 waveform plot 2224. Each lead shows two waveforms: a solid line representing individuals without hypertrophic cardiomyopathy and a dashed line representing individuals with hypertrophic cardiomyopathy. A key 2226 indicates the line styles used to differentiate between individuals without hypertrophic cardiomyopathy (solid line) and those with hypertrophic cardiomyopathy (dashed line). Each waveform plot displays voltage (in millivolts, mV) on the vertical axis and time on the horizontal axis (in seconds). The waveforms demonstrate characteristic differences in ECG patterns between individuals with and without hypertrophic cardiomyopathy across all leads.

FIG. 23 illustrates an example 2300 of median waveform reconstructions for centroids reflecting individuals without and with rheumatoid arthritis. A 12-lead electrocardiogram is shown, including a lead I waveform plot 2302, a lead aVR waveform plot 2304, a lead V1 waveform plot 2306, a lead V4 waveform plot 2308, a lead II waveform plot 2310, a lead aVL waveform plot 2312, a lead V2 waveform plot 2314, a lead V5 waveform plot 2316, a lead III waveform plot 2318, a lead aVF waveform plot 2320, a lead V3 waveform plot 2322, and a lead V6 waveform plot 2324. Each lead shows two waveforms: a solid line representing individuals without rheumatoid arthritis and a dashed line representing individuals with rheumatoid arthritis. A key 2326 indicates the line styles used to differentiate between individuals without rheumatoid arthritis (solid line) and those with rheumatoid arthritis (dashed line). Each waveform plot displays voltage (in millivolts, mV) on the vertical axis and time on the horizontal axis (in seconds). The waveforms demonstrate characteristic differences in ECG patterns between individuals with and without rheumatoid arthritis across all leads.

### Patient Report Card Prototype

To demonstrate the potential for the ECG to serve as a digital biomarker for disease status, a prototype of an ECG-based patient report for select Phecodes was generated (Table 4). In this illustrative example, an ECG from a 65-year-old female is projected onto the phenotype vectors for select circulatory system diseases, and the positions relative to the whole cohort along vectors from the disease-negative to disease-positive centroids are reported. These diseases were selected based on clinical relevance and the potential to cause substantial morbidity if undetected, including myocardial infarction, ventricular tachycardia, and heart failure.

### Discussion

The use of autoencoder deep learning models to encode and reconstruct 12- and single-lead ECGs in order to generate a multidimensional latent space encoding ECG waveform features with demonstrable relevance to risk of disease across the spectrum of human conditions is highlighted. Indeed, the results demonstrate robust associations between ECG waveform patterns and conditions spanning the full spectrum of human disease. When compared to standard ECG intervals, the autoencoder-based latent space models reveal a substantially greater number of associations, suggesting that the rich representations afforded by the architectural complexity of the deep learning model provide more information about disease status than routinely ascertained ECG intervals.

Autoencoder models have been used previously in fields such as linguistics and image processing to better understand input information. Prior studies on image representation have demonstrated that attribute labeling (e.g., headshot photograph categorized as “wearing glasses” versus “no glasses”) can aid interpretation of the machine-learned latent space, as images with similar attributes form interpretable semantic clusters. Cluster centroids are spatially defined using attribute vectors, and any unlabeled image projection can be relationally defined by its position along the vector, based solely on the image characteristics interpreted by the model. These studies further demonstrate that the reconstruction of an input image (e.g., person without glasses) can be modified by movement along an attribute vector, generating a novel image with features that are more aligned with the corresponding centroid pair (e.g., the same photograph reconstructed with glasses). Although autoencoders have recently been applied to the ECG primarily in efforts to create more interpretable deep learning pipelines, this work extends prior models by leveraging attribute, or in this case phenotype, labeling to explore disease-state information across the full spectrum of human conditions, to define a range of both cardiac and noncardiac conditions detectable by the ECG.

By applying autoencoder-based technology to clinical ECG data, the results yield several implications. First, it is demonstrated how latent space modeling can be used for the discovery of novel information contained within the ECG. Specifically, phenotype labeling was applied to explore disease-state information contained in 12- and single-lead ECGs. By reconstructing median waveforms from latent space centroids, disease-based patterns identified by deep learning models are visually represented, which in some cases confirm expectations and in other cases reflect subtle waveform manifestations potentially below the level of human detection. Furthermore, although the primary aim was to investigate the detection of existing disease, secondary analyses suggest that autoencoder representations additionally possess utility for the prediction of incident disease. Thus, samples acquired prior to and after a given diagnosis may facilitate the derivation of “disease progression vectors,” allowing visualization of waveform evolution over time.

Second, while ECG-based deep learning models have been developed previously, studies have predominantly focused on disease-specific risk prediction within the cardiovascular system. In taking a more global analytic approach, ECG-based classification of non-cardiac disease has been identified. Specific conditions for which further study may be particularly high yield include diseases not classically associated with ECG findings but each independently supported by prior studies (e.g., type 2 diabetes, sleep apnea, chronic liver disease/cirrhosis, and renal failure), as well as diseases with previously undescribed associations (e.g., fever of unknown origin, tobacco use disorder). Improvements in disease discrimination were particularly enriched for conditions commonly encountered during critical illness with clear ECG manifestations (e.g., acid-base disorders, sepsis, shock, arrhythmias), consistent with recent work demonstrating the particular value of ECG-based deep learning in critical care populations.

Third, the potential for personalized and scalable disease detection with ECG-based latent space modeling was demonstrated. Using data derived from large samples, a complex architectural environment informed by disease status was constructed, in which each ECG encoding represents a single individual and occupies a unique position in the latent space. Projection of new ECGs from independent individuals can therefore be used to generate likelihoods of disease at scale. As latent space modeling approaches are refined with data from larger and more diverse samples, the utility for disease-based classification will grow. The approach outlined in the Example Application will have particular value for detecting diseases in which screening may be cumbersome, inaccurate, or expensive, and for which early disease manifestations may be highly morbid (e.g., aortic aneurysm and valvular heart disease). Although this approach possesses several potential advantages when compared to training a large number of individual disease-specific classifiers (e.g., the requirement to train and implement only a single model) or a single large multi-task disease classifier (e.g., simpler architecture, lower requirements on model capacity, no dependence on a varying frequency of disease labels), alternative modeling strategies may be used. This unsupervised modeling strategy may be better suited to broad risk profiling at scale. Indeed, the robust performance of the single-lead model for prevalent disease detection highlights the potential utility for screening large populations, particularly given the widespread emergence of consumer-based wearable and handheld devices with ECG recording capabilities.

The Example Application presents one implementation of the techniques described herein. Since the primary aim was to develop and apply an autoencoder-based approach to systematically identify conditions whose presence may be detectable on the ECG, phenotype vectors were derived among individuals with known disease, and of varying durations. Although training among individuals with known disease may enrich for more severe cases, such an approach is customary in the development of disease classification models. Prospective validation would be required to confirm performance among individuals in whom disease status is unknown at the time of ECG acquisition. Single linear probes were used to define phenotype vectors. Improvements in discrimination with the incorporation of autoencoder information were substantial in MGH and BWH but more modest in the UK Biobank, which is likely due to differences in sample composition (i.e., healthier) and lower event rates, although lower autoencoder reconstruction accuracy or varying informativeness of ECG features across datasets are additional potential contributing factors. To standardize the phase of the cardiac cycle and minimize the effects of artifacts, ECGs were encoded as median waveforms. Such an approach may result in the loss of some information related to R-R regularity and subtle beat-to-beat changes in morphology, including ectopic beats. However, it is demonstrated that the median samples encode heart rate information. The approach was not compared to the analysis of the raw median ECG beat. However, the autoencoder approach retains specific advantages over use of the raw ECG beat (e.g., flexibility, computational efficiency), and possesses the potential to extend to modalities where use of the raw signal may be computationally infeasible (e.g., multi-modality imaging). The patient report card prototype is offered as a demonstration of the concept of how an ECG autoencoder model could be applied to broadly classify risk of present but potentially undiagnosed disease. Although the models were adjusted for basic factors likely to confound all potential disease associations (e.g., age, sex, race), given the number of associations tested, residual confounding cannot be excluded or the degree to which associations may be driven by clinical factors encoded by the ECG quantified, and therefore the findings should not be used to infer causal relations. However, median samples are plotted that demonstrate changes consistent with clinical expectations. The findings are subject to selection bias due to the requirement for a 12-lead ECG obtained for clinical purposes in MGH and BWH. However, the continued value of the autoencoder latent space in the UK Biobank is noted, where ECGs were obtained prospectively as part of a research protocol.

It is demonstrated how latent space modeling can be used to organize and better understand disease-related information contained within currently available diagnostics. The corresponding analysis demonstrates that the ECG waveform contains a wealth of disease-state information beyond the circulatory system, with the potential to detect hundreds of prevalent conditions and even stratify the risk of incident disease.

### Methods

### Study Subjects

The study utilized three nonoverlapping, independent datasets spanning over 150,000 individuals, each containing individual-level demographic and clinical information, including 12-lead ECGs. Two datasets were derived from the Community Care Cohort Project (C3PO), a previously established cohort comprising over 500,000 adults aged ≥18 years who received longitudinal primary care at one of eleven hospitals within the Mass General Brigham network, linked by a common EHR data warehouse. The C3PO datasets included a cohort from the Massachusetts General Hospital (MGH-C3PO dataset) and a cohort from the Brigham and Women's Hospital (BWH-C3PO dataset). The third, external dataset was derived from the UK Biobank, a prospective community-based cohort study comprising adults aged 40-60 years at enrollment between 2006-2010 from the United Kingdom. The analysis included subsets of individuals in each dataset with at least one 12-lead ECG performed within three years prior to the start of follow-up (C3PO) or who had a 12-lead ECG performed during at least one study visit (UK Biobank). See, for example, FIG. 14B.

### ECG Autoencoder Model and Latent Space Derivation

Densely connected convolutional autoencoders were trained to encode and reconstruct 12- and single-lead ECGs. Autoencoders consist of an encoder, which maps a high-dimensional input into a lower-dimensional latent space, and a decoder, which reconstructs the original data from the latent space representation (see, for example, FIG. 2). Autoencoders are trained to encode variance present within the original data into the latent space, which encourages the model to minimize differences between the original data and its reconstruction. Both the 12-lead and single-lead autoencoders were trained and validated using subsets of ECGs from the MGH-C3PO cohort. The models were then tested in an MGH-C3PO holdout set as well as two true holdout datasets, including BWH-C3PO and the UK Biobank.

To standardize the phase of the cardiac cycle across all individuals while minimizing the effects of signal artifact (e.g., baseline drift, transient noise), ECGs were encoded as median waveforms by segmenting 10-second ECG recordings into 1200 millisecond windows, sampling 600 voltage timepoints per window, and performing piecewise linear interpolation to generate R-R adjusted medians. Median waveforms therefore represent the aggregate morphology of at least one cardiac cycle from each lead (FIGS. 15A and 15B). When assessed in 1,000 randomly sampled individuals from the UK Biobank, heart rate was easily recovered from the median waveform (r=0.94, 95% CI 0.93-0.95), demonstrating little loss in heart rate information. The 12-lead model utilized median waveforms generated from all available leads (i.e., 12 waveforms per ECG), while the single-lead model utilized only the median waveform generated from lead I. In the following analysis, the term ECG generally refers to the median waveform.

Models were trained using one-dimensional convolutions over voltage-time series, corresponding to 7,200 voltage timepoints for 12-lead ECGs and 600 voltage timepoints for single leads. For ECGs with incomplete voltage data (i.e., less than 10 seconds recorded from each lead), zero padding was used, converting non-available data into zeros. The mean squared error per voltage timepoint across the full ECG was minimized, as demonstrated in Eq. (1):

$${Lv}_{ECG} , {v}_{AE} = {v}_{ECG} - {v}_{AE}^{2}$$

In C3PO, ECGs were excluded if the acquisition date was greater than three years prior to the start of clinical follow up, defined for each individual as the time of the second primary care visit of the earliest qualifying pair. Only one ECG per individual was represented; for patients with multiple ECGs, the most recent was used.

The neural net architecture was a variant of Densenet, featuring several densely connected convolutional blocks operating at different time resolutions. Architecture hyperparameters, including width, depth, activation, normalization, and regularization were chosen via Bayesian hyperparameter optimization.

To assess the accuracy of reconstruction in each of the three datasets, voltages were pooled across test set ECGs and compared to the voltages generated from reconstructions. For each dataset, the average per-voltage Pearson correlation coefficient and the 95% confidence interval based on 1000 bootstrap resamplings were assessed. This method was selected, as opposed to comparing per-person pooled voltages across test set and reconstructed ECGs, to assess the model's global performance in ECG reconstruction rather than its performance reproducing ECGs from specific individuals within the datasets (FIGS. 15A and 15B).

### Phenotype Definitions

ICD codes in each of the three datasets were mapped to a publicly available Phecode library. Phecodes distinguish cases from controls using hierarchical groupings of ICD 9 and 10 codes to better define clinically meaningful disease phenotypes. Only prevalent Phecodes were used, i.e., all corresponding ICD codes had been entered into the patient's chart prior to the ECG acquisition date. For certain Phecodes, participants without that Phecode but with very similar ICD codes were excluded from serving as controls to avoid biasing results, as described previously (e.g., in association testing for the myocardial infarction case group, patients were removed from serving as controls if they had ICD codes corresponding to a list of disease exclusions, including angina or other evidence of ischemic heart disease).

### Phenotype Vector Derivation

If a given disease, represented by a Phecode, has a significant impact on the ECG, ECG encodings from individuals with the disease are expected to distribute to a different location in the autoencoder-derived latent space relative to ECG encodings from individuals without the disease. In contrast, if the disease has little impact on the ECG, or if the ECG encoding does not adequately capture disease-relevant features, then no significant relationship is expected between the position of the ECG encoding in latent space and the presence or absence of disease.

To quantify this expectation, the highest density of ECG encodings labeled as having the disease (“disease-positive centroid” for cases) and the highest density of ECG encodings labeled as not having the disease (“disease-negative centroid” for controls) were defined. Each Phecode is therefore spatially represented by its centroid pair, and the line that connects them is referred to as the phenotype vector (see, for example, FIG. 8). Uniform manifold approximation and projections (UMAPs) were also generated to visually assess ECG encodings for age, sex, and body mass index (FIGS. 16A and 16B).

During autoencoder training/latent space derivation, a subset of ECGs from the MGH-C3PO dataset was labeled as disease cases or controls based on the presence or absence of the corresponding Phecode in the patient's EHR. ECG encodings from the derivation set were then used to define centroid pairs and phenotype vectors for each Phecode within the latent space.

### ECG Projections

Any ECG encoded within the latent space, including encodings from unlabeled samples, can be projected onto any phenotype vector, and the relative position along the phenotype vector can be used to assess how closely related the unlabeled ECG encoding is to encodings that were used to define the disease positive centroid for that Phecode.

To externally validate the approach to classify the presence of disease in samples independent of autoencoder and phenotype vector derivation, after deriving phenotype vectors in the MGH-C3PO training set, unlabeled ECG encodings from three test sets independent of autoencoder and phenotype vector derivation were projected onto the phenotype vectors: a) the 50% holdout component of the MGH-C3PO cohort, b) BWH-C3PO, and c) the UK Biobank. There was no modification of the autoencoder or the phenotype vectors in the application of the model to the test sets.

The entire space of ECG encodings was scaled to have norm and standard deviation of one. Each phenotype vector was normalized to have length one. The high-dimensional spatial relatedness of each ECG and Phecode was quantified by each sample's component in the direction of a given phenotype vector. As illustrated in FIG. 9, for example, each ECG encoding (“ECG embedding,” ECGᵢ) projects onto each phenotype vector, Vₚ. The projected component, (“componentᵢₚ”) is calculated from the angle between the ECG encoding and the phenotype vector, scaled by the length of the ECG, as displayed in Eq. (2). Thus, the projected component signifies the latent space position of a single individual along a single phenotype vector and therefore represents a disease-specific “vector component score”.

$${Component}_{ip} = \frac{{ECG}_{i} \cdot {V}_{p}}{‖ {V}_{p} ‖} = ‖ {ECG}_{i} ‖ cos ( {θ}_{ip} )$$

Analogous methods were used for both 12- and single-lead models. In the single-lead model, the autoencoder-derived latent space, phenotype vector derivation, and ECG projections were based only on median waveform data derived from lead I.

### Association Testing by Latent Space PheWAS

For both 12- and single-lead models, a PheWAS was performed in each dataset using a logistic regression model to assess the strength of the relationship between a given disease-specific vector component score and the presence of the target disease state (using Phecode presence or absence as the outcome variable). In this way, the odds ratio represents the adjusted odds for the presence of disease for every 1-point increase in the vector component score. For the UK Biobank dataset, the model was adjusted for age, sex, and race. For the MGH-C3PO holdout set and the BWH-C3PO dataset, the model was additionally adjusted for ECG acquisition date and the amount of zero padding (as the degree of missing voltage data may represent other confounders such as ECG quality, ECG machine used, hospital location, etc.).

For both latent space models, a fixed-effects inverse variance weighted meta-analysis was performed, filtering for Phecodes that were present in at least two datasets with at least 100 combined cases. Coefficients corresponding to each phenotype vector were pooled across datasets. For comparison, a separate model based on ECG intervals, including the PR interval, QRS duration, and QT interval, was generated. An intervals-based model was chosen as the comparator because it utilizes routinely ascertained, standardized, and automated measurements known to have disease-based prognostic implications. An intervals PheWAS was performed in each dataset and results were meta-analyzed. The number of significant associations between the latent space models and the ECG intervals model was then compared, using a Bonferroni-corrected two-sided p-value of 0.05 divided by the number of common Phecodes across all meta-analyses. For the intervals model, a result was considered significant if the p-value for any interval met the significance threshold. Metanalyses were performed using the R package meta. Python was used to create a visual summary of meta-analyzed data, grouped according to disease category. To estimate the potential added value for disease detection, the difference in discrimination of individual Phecodes was assessed by calculating the difference in the area under the curve (AUC) for the logistic regression models described above with versus without ECG vector component scores, among Phecodes with Bonferroni-corrected significant associations with the component model in the primary meta-analysis. In a secondary analysis, AUC estimation was repeated using models comparing the ECG component vectors to standard ECG intervals (PR interval, RR interval, QRS duration, and QT interval).

Although the primary goal was to leverage latent space models to assess the degree to which existing diseases may be detectable using ECG waveform, the potential for the ECG latent space to predict the risk of incident disease was explored using an analogous approach to that outlined above, except using Cox proportional hazards models rather than logistic regression models. Individuals with a Phecode diagnosis present at baseline (i.e., start of follow-up) were excluded from incident analyses of that Phecode, and a Phecode event was defined as the first instance of any component of the given Phecode definition. In the C3PO datasets, person-time began at the start of follow-up and ended at the earliest of an outcome event, death, last encounter in the EHR, or Aug. 31, 2019. In the UK Biobank, person-time began at the ECG study visit and ended at the earliest of outcome event, death, or last follow-up. The date of the last follow-up in the UK Biobank was Mar. 31, 2021 for individuals enrolled in England and Scotland, and Feb. 28, 2018 for individuals enrolled in Wales.

### Phenotype-Based ECG Reconstructions

To better understand which ECG features may have contributed to Phecode segregation in latent space, disease-specific median waveforms were generated. Specifically, ECGs were decoded from disease-positive centroids and the resultant median waveforms were overlayed on ECGs decoded from the corresponding disease-negative centroids. See, for example, FIGS. 20-23.

### Patient Report Card

One advantage of latent space modeling is the ability to incorporate an enormous amount of ECG- and EHR-based data in a multidimensional environment. The multidimensional nature of the model allows for simultaneous assessment of an ECG encoding's proximity to all Phecode centroids. To demonstrate the associated potential for personalized and scalable disease reporting, the position of an ECG encoding for a single individual was converted into an estimated probability of disease. The probability of disease is estimated as the disease prevalence among all individuals in the MGH-C3PO test set with a vector component value greater than or equal to the individual's value. The exact method was used to estimate 95% confidence intervals.

TABLE 1
Analysis sample characteristics

| | Mean (± SD) or n (%) | | |
|---|---|---|---|
| | MGH (n = 60,140) | BWH | UK Biobank |
| | Internal | (n = 46,027) | (n = 35,070) |
| | Development | External | External |
| Covariate | and Test sample* | Test Sample | Test Sample |
| Age at ECG | 51.6 (17.2) | 54.8 (15.9) | 63.6 (7.6) |
| Female sex | 31,366 | 27,345 | 18,144 |
| | (52.2%) | (59.4%) | (51.7%) |
| Race | | | |
| White | 46,127 (76.7%) | 30,973 (67.3%) | 33,924 (96.7%) |
| Black | 3,876 (6.4%) | 6,066 (13.2%) | 234 (0.7%) |
| Asian | 2,759 (4.6%) | 1,177 (2.6%) | 472 (1.4%) |
| Hispanic | 3,201 (5.3%) | 3,724 (8.1%) | |
| Other | 2,542 (4.2%) | 1,785 (3.9%) | 339 (1.0%) |
| Not reported | 1,635 (2.7%) | 2,302 (5.0%) | 101 (0.3%) |
| Year ECG performed | 2005 (2001, | 2008 (2003, | 2018 (2016, |
| (median, IQR) | 2012) | 2013) | 2019) |

*Autoencoder model split samples in MGH (train = 35,245, validate = 10,152, test = 14,743). No individuals in the test set were used for model training.
ECG = electrocardiogram;
SD = standard deviation

TABLE 2
Top associations by effect size across disease category

| | | | |
|---|---|---|---|
| | N | Odds ratio | |
| Disease grouping* | events | (95% CI)^{†} | p |

Circulatory system

| | | | |
|---|---|---|---|
| Cardiac defibrillator in situ | 514 | 2.18 (2.04-2.32) | 6.83 × 10⁻¹²⁴ |
| Bundle branch block | 1679 | 2.16 (2.07-2.25) | 1.11 × 10⁻³⁰³ |
| Other hypertrophic | 127 | 2.13 (1.78-2.54) | 4.21 × 10⁻¹⁷ |
| cardiomyopathy | | | |
| Left bundle branch block | 927 | 2.12 (2.03-2.22) | 1.13 × 10⁻²⁴⁴ |
| Right bundle branch block | 868 | 2.08 (1.98-2.18) | 5.80 × 10⁻¹⁹⁷ |

Congenital anomalies

| | | | |
|---|---|---|---|
| Cardiac shunt/heart | 1303 | 1.32 (1.25-1.39) | 3.16 × 10⁻²⁶ |
| septal defect | | | |
| Cardiac congenital anomalies | 2876 | 1.22 (1.18-1.26) | 7.76 × 10⁻⁴⁰ |
| Cardiac and circulatory | 3632 | 1.21 (1.18-1.24) | 2.38 × 10⁻⁴⁰ |
| congenital anomalies | | | |
| Spondylolisthesis, congenital | 478 | 1.17 (1.09-1.25) | 1.28 × 10⁻⁰⁵ |

Dermatologic

| | | | |
|---|---|---|---|
| Decubitus ulcer | 479 | 1.44 (1.33-1.55) | 2.05 × 10⁻²⁰ |
| Chronic ulcer of leg or foot | 1410 | 1.36 (1.30-1.42) | 1.29 × 10⁻⁴⁷ |
| Chronic ulcer of skin | 2155 | 1.30 (1.25-1.34) | 8.35 × 10⁻⁵³ |
| Cellulitis and abscess | 1204 | 1.29 (1.22-1.36) | 7.20 × 10⁻²² |
| of foot, toe | | | |
| Cellulitis and abscess of trunk | 1513 | 1.28 (1.22-1.33) | 3.81 × 10⁻³¹ |

Digestive

| | | | |
|---|---|---|---|
| Portal hypertension | 426 | 1.51 (1.40-1.63) | 4.01 × 10⁻²⁸ |
| Acute and subacute | 251 | 1.40 (1.25-1.56) | 2.04 × 10⁻⁰⁹ |
| necrosis of liver | | | |
| Complications of | 181 | 1.36 (1.19-1.54) | 3.60 × 10⁻⁰⁶ |
| gastrostomy, colostomy | | | |
| and enterostomy | | | |
| Liver replaced by transplant | 162 | 1.35 (1.18-1.53) | 9.43 × 10⁻⁰⁶ |
| Cirrhosis of liver without | 1078 | 1.33 (1.28-1.39) | 7.05 × 10⁻³⁸ |
| mention of alcohol | | | |

Endocrine/Metabolic

| | | | |
|---|---|---|---|
| Cachexia | 176 | 1.85 (1.64-2.09) | 4.30 × 10⁻²³ |
| Alkalosis | 224 | 1.76 (1.58-1.95) | 1.27 × 10⁻²⁶ |
| Diabetes type 1 with | 158 | 1.75 (1.54-1.98) | 1.91 × 10⁻¹⁸ |
| peripheral circulatory | | | |
| disorders | | | |
| Type 1 diabetes with | 297 | 1.60 (1.47-1.75) | 1.54 × 10⁻²⁵ |
| ophthalmic manifestations | | | |
| Acidosis | 1239 | 1.52 (1.45-1.58) | 1.08 × 10⁻⁸⁵ |

Genitourinary

| | | | |
|---|---|---|---|
| Nephritis and nephropathy | 481 | 1.46 (1.37-1.56) | 5.80 × 10⁻²⁹ |
| in diseases classified | | | |
| elsewhere | | | |
| Renal dialysis | 1001 | 1.38 (1.32-1.44) | 1.92 × 10⁻⁴⁷ |
| Acute renal failure | 3700 | 1.37 (1.34-1.40) | 1.43 × 10⁻¹⁵³ |
| End stage renal disease | 709 | 1.36 (1.29-1.44) | 4.39 × 10⁻²⁹ |
| Kidney replaced by transplant | 734 | 1.35 (1.28-1.43) | 3.39 × 10⁻²⁶ |

Hematopoietic

| | | | |
|---|---|---|---|
| Anemia in chronic | 447 | 1.51 (1.40-1.63) | 5.40 × 10⁻²⁷ |
| kidney disease | | | |
| Secondary thrombocytopenia | 610 | 1.51 (1.41-1.61) | 2.32 × 10⁻³⁵ |
| Deficiency anemias | 367 | 1.33 (1.22-1.46) | 4.84 × 10⁻¹⁰ |
| Aplastic anemia | 712 | 1.31 (1.24-1.39) | 4.52 × 10⁻²¹ |
| Acquired hemolytic anemias | 249 | 1.30 (1.18-1.44) | 6.93 × 10⁻⁰⁸ |

Infectious diseases

| | | | |
|---|---|---|---|
| Gram positive septicemia | 388 | 1.57 (1.45-1.71) | 8.88 × 10⁻²⁸ |
| Infection with drug- | 468 | 1.42 (1.32-1.52) | 1.28 × 10⁻²¹ |
| resistant microorganisms | | | |
| Bacteremia | 1373 | 1.41 (1.35-1.46) | 6.06 × 10⁻⁶⁸ |
| Septicemia | 2585 | 1.40 (1.36-1.44) | 1.84 × 10⁻¹¹⁴ |
| Methicillin sensitive | 892 | 1.39 (1.32-1.46) | 6.48 × 10⁻³⁶ |
| Staphylococcus aureus | | | |

Injuries and poisonings

| | | | |
|---|---|---|---|
| Septic shock | 241 | 1.48 (1.35-1.63) | 1.78 × 10⁻¹⁶ |
| Systemic inflammatory | 151 | 1.45 (1.25-1.69) | 1.33 × 10⁻⁰⁶ |
| response syndrome (SIRS) | | | |
| Subarachnoid | 162 | 1.44 (1.23-1.68) | 3.21 × 10⁻⁰⁶ |
| hemorrhage (injury) | | | |
| Sepsis | 926 | 1.43 (1.36-1.50) | 8.79 × 10⁻⁴³ |
| Sepsis and SIRS | 1028 | 1.42 (1.35-1.49) | 7.02 × 10⁻⁴⁵ |

Mental disorders

| | | | |
|---|---|---|---|
| Alcoholic liver damage | 683 | 1.48 (1.40-1.56) | 2.80 × 10⁻⁴⁶ |
| Delirium due to | 1375 | 1.39 (1.34-1.45) | 1.13 × 10⁻⁵⁷ |
| conditions classified | | | |
| elsewhere | | | |
| Altered mental status | 1783 | 1.32 (1.28-1.37) | 8.45 × 10⁻⁵² |
| Alcoholism | 4669 | 1.31 (1.28-1.34) | 2.71 × 10⁻¹²⁵ |
| Alcohol-related disorders | 5982 | 1.28 (1.26-1.31) | 1.71 × 10⁻¹³⁰ |

Musculoskeletal

| | | | |
|---|---|---|---|
| Panniculitis | 109 | 1.47 (1.25-1.74) | 5.12 × 10⁻⁰⁶ |
| Osteitis deformans and | 273 | 1.33 (1.22-1.45) | 9.86 × 10⁻¹¹ |
| osteopathies associated | | | |
| with other disorders | | | |
| classified elsewhere | | | |
| Infective connective | 159 | 1.32 (1.18-1.48) | 1.96 × 10⁻⁰⁶ |
| tissue disorders | | | |
| Acute osteomyelitis | 383 | 1.32 (1.22-1.42) | 1.32 × 10⁻¹² |
| Unspecified osteomyelitis | 812 | 1.26 (1.20-1.33) | 4.84 × 10⁻²⁰ |

Neoplasms

| | | | |
|---|---|---|---|
| Hodgkin's disease | 450 | 1.38 (1.28-1.49) | 6.23 × 10⁻¹⁶ |
| Bone marrow or stem | 327 | 1.30 (1.19-1.42) | 4.18 × 10⁻⁰⁹ |
| cell transplant | | | |
| Secondary malignancy | 546 | 1.16 (1.09-1.23) | 1.87 × 10⁻⁰⁶ |
| of bone | | | |
| Secondary malignancy | 686 | 1.15 (1.09-1.22) | 2.49 × 10⁻⁰⁶ |
| of respiratory organs | | | |
| Cancer of other lymphoid, | 1882 | 1.14 (1.11-1.18) | 4.63 × 10⁻¹⁷ |
| histiocytic tissue | | | |

Neurological

| | | | |
|---|---|---|---|
| Coma | 280 | 1.42 (1.27-1.58) | 1.14 × 10⁻¹⁰ |
| Peripheral autonomic | 285 | 1.32 (1.21-1.43) | 7.19 × 10⁻¹¹ |
| neuropathy | | | |
| Cerebral degeneration, | 373 | 1.30 (1.19-1.42) | 6.35 × 10⁻⁰⁹ |
| unspecified | | | |
| Encephalitis, non-infectious | 256 | 1.29 (1.17-1.43) | 7.74 × 10⁻⁰⁷ |
| Other paralytic syndromes | 556 | 1.27 (1.18-1.37) | 1.07 × 10⁻⁰⁹ |

Pregnancy complications

| | | | |
|---|---|---|---|
| Hypertension complicating | 1282 | 1.15 (1.09-1.22) | 1.55 × 10⁻⁰⁷ |
| pregnancy, childbirth, | | | |
| and the puerperium | | | |

Respiratory

| | | | |
|---|---|---|---|
| Obstructive chronic bronchitis | 1182 | 1.71 (1.62-1.80) | 8.56 × 10⁻⁹¹ |
| Respiratory failure | 1881 | 1.65 (1.60-1.71) | 9.38 × 10⁻¹⁷⁴ |
| Emphysema | 2471 | 1.64 (1.58-1.71) | 1.05 × 10⁻¹³⁸ |
| Pneumonitis due to | 919 | 1.60 (1.52-1.68) | 9.01 × 10⁻⁷⁶ |
| inhalation of food or vomitus | | | |
| Pulmonary congestion | 3653 | 1.59 (1.55-1.63) | 1.24 × 10⁻²⁴⁵ |
| and hypostasis | | | |

Sense organs

| | | | |
|---|---|---|---|
| Other nondiabetic retinopathy | 237 | 1.24 (1.13-1.36) | 8.18 × 10⁻⁰⁶ |
| Blindness and low vision | 976 | 1.15 (1.08-1.23) | 2.16 × 10⁻⁰⁵ |
| Dizziness and giddiness | 14603 | 1.05 (1.04-1.07) | 3.94 × 10⁻¹³ |
| (Light-headedness | | | |
| and vertigo) | | | |

Symptoms

| | | | |
|---|---|---|---|
| Cardiogenic shock | 287 | 1.71 (1.57-1.87) | 5.34 × 10⁻³³ |
| Shock | 656 | 1.60 (1.51-1.70) | 9.88 × 10⁻⁵⁴ |
| Gangrene | 302 | 1.49 (1.36-1.63) | 1.47 × 10⁻¹⁷ |
| Rhabdomyolysis | 185 | 1.28 (1.15-1.42) | 7.23 × 10⁻⁰⁶ |
| Fever of unknown origin | 13114 | 1.18 (1.16-1.19) | 5.46 × 10⁻¹³⁶ |

*Displayed are significant associations with the top 5 largest effect sizes within each disease category. In cases where there are fewer than 5 significant associations, all significant associations are shown.
^{†}Odds ratios are presented per 1-point increase in vector component score (see text)

TABLE 3
Top associations by effect size across disease category
for incident disease.

| | | | |
|---|---|---|---|
| | N | Hazard ratio | |
| Disease grouping* | events | (95% CI)^{†} | p |

Circulatory system

| | | | |
|---|---|---|---|
| Cardiac defibrillator in situ | 322 | 1.80 (1.65-1.95) | 2.18 × 10⁻⁶⁵ |
| Right bundle branch block | 924 | 1.77 (1.70-1.84) | 1.39 × 10⁻²³3 |
| Hypertrophic obstructive | 222 | 1.69 (1.63-1.75) | 2.92 × 10⁻¹⁹ |
| cardiomyopathy | | | |
| Left bundle branch block | 788 | 1.64 (1.57-1.72) | 2.84 × 10⁻¹³² |
| Bundle branch block | 1674 | 1.61 (1.53-1.70) | 3.15 × 10⁻¹⁷⁸ |

Congenital anomalies

| | | | |
|---|---|---|---|
| Valvular heart disease/ | 525 | 1.25 (1.17-1.33) | 5.94 × 10⁻¹² |
| heart chambers | | | |
| Cardiac congenital anomalies | 2158 | 1.24 (1.20-1.28) | 1.09 × 10⁻⁴⁷ |
| Cardiac and circulatory | 2659 | 1.22 (1.19-1.25) | 3.04 × 10⁻⁴⁸ |
| congenital anomalies | | | |
| Cardiac shunt/heart | 1052 | 1.10 (1.06-1.15) | 1.40 × 10⁻⁰⁶ |
| septal defect | | | |

Dermatologic

| | | | |
|---|---|---|---|
| Decubitus ulcer | 1204 | 1.26 (1.22-1.31) | 1.88 × 10⁻³⁴ |
| Chronic ulcer of leg or foot | 2218 | 1.24 (1.21-1.28) | 1.15 × 10⁻⁶³ |
| Chronic ulcer of skin | 3522 | 1.22 (1.19-1.24) | 7.60 × 10⁻⁸¹ |
| Chronic ulcer of | 1062 | 1.19 (1.15-1.24) | 1.21 × 10⁻²⁰ |
| unspecified site | | | |
| Cellulitis and | 712 | 1.18 (1.13-1.23) | 3.64 × 10⁻¹⁵ |
| abscess of foot, toe | | | |

Digestive

| | | | |
|---|---|---|---|
| Chronic passive | 187 | 1.35 (1.20-1.51) | 5.24 × 10⁻⁰⁷ |
| congestion of liver | | | |
| Liver abscess and | 546 | 1.28 (1.22-1.35) | 1.09 × 10⁻²² |
| sequelae of chronic | | | |
| liver disease | | | |
| Acute and subacute | 340 | 1.28 (1.19-1.38) | 1.34 × 10⁻¹⁰ |
| necrosis of liver | | | |
| Portal hypertension | 235 | 1.27 (1.18-1.36) | 9.71 × 10⁻¹¹ |
| Lesions of stomach | 248 | 1.23 (1.13-1.34) | 1.07 × 10⁻⁰⁶ |
| and duodenum | | | |

Endocrine/Metabolic

| | | | |
|---|---|---|---|
| Alkalosis | 366 | 1.37 (1.27-1.47) | 1.82 × 10⁻¹⁶ |
| Cachexia | 494 | 1.35 (1.27-1.44) | 5.58 × 10⁻²² |
| Fluid overload | 1916 | 1.32 (1.28-1.35) | 4.09 × 10⁻⁸⁵ |
| Type 2 diabetes with | 1101 | 1.31 (1.27-1.36) | 1.69 × 10⁻⁵⁴ |
| renal manifestations | | | |
| Disorders of | 1503 | 1.31 (1.27-1.35) | 2.42 × 10⁻⁶² |
| magnesium metabolism | | | |

Genitourinary

| | | | |
|---|---|---|---|
| Acute renal failure | 5893 | 1.27 (1.25-1.29) | 1.92 × 10⁻²¹⁴ |
| Kidney replaced by transplant | 189 | 1.26 (1.15-1.38) | 3.99 × 10⁻⁰⁷ |
| Nephritis and nephropathy | 350 | 1.26 (1.19-1.33) | 8.25 × 10⁻¹⁵ |
| in diseases classified elsewhere | | | |
| Renal dialysis | 1203 | 1.26 (1.22-1.30) | 2.55 × 10⁻⁴² |
| Disorders resulting | 570 | 1.26 (1.20-1.32) | 7.97 × 10⁻²² |
| from impaired renal function | | | |

Hematopoietic

| | | | |
|---|---|---|---|
| Defibrination syndrome | 153 | 1.43 (1.24-1.65) | 9.00 × 10⁻⁰⁷ |
| Secondary thrombocytopenia | 993 | 1.32 (1.27-1.38) | 1.11 × 10⁻⁴³ |
| Anemia in chronic | 858 | 1.24 (1.19-1.29) | 3.29 × 10⁻²⁶ |
| kidney disease | | | |
| Hemorrhagic disorder | 333 | 1.22 (1.14-1.29) | 2.67 × 10⁻¹⁰ |
| due to intrinsic | | | |
| circulating anticoagulants | | | |
| Acute posthemorrhagic anemia | 2501 | 1.21 (1.18-1.24) | 7.64 × 10⁻⁵¹ |

Infectious diseases

| | | | |
|---|---|---|---|
| Methicillin resistant | 670 | 1.27 (1.20-1.35) | 7.94 × 10⁻¹⁷ |
| Staphylococcus aureus | | | |
| Septicemia | 3553 | 1.27 (1.24-1.29) | 2.51 × 10⁻¹²⁴ |
| Bacteremia | 1802 | 1.27 (1.23-1.30) | 4.50 × 10⁻⁶² |
| Gram positive septicemia | 328 | 1.25 (1.17-1.35) | 8.96 × 10⁻¹⁰ |
| Intestinal infection | 1132 | 1.25 (1.20-1.30) | 3.24 × 10⁻²⁹ |
| due to C. difficile | | | |

Injuries and poisonings

| | | | |
|---|---|---|---|
| Septic shock | 1088 | 1.34 (1.29-1.39) | 1.63 × 10⁻⁵⁶ |
| Complication of | 146 | 1.32 (1.18-1.47) | 1.31 × 10⁻⁰⁶ |
| amputation stump | | | |
| Anticoagulants causing | 255 | 1.30 (1.20-1.41) | 3.07 × 10⁻¹¹ |
| adverse effects | | | |
| Sepsis | 2765 | 1.30 (1.27-1.33) | 2.84 × 10⁻¹¹⁸ |
| Sepsis and SIRS | 2888 | 1.30 (1.27-1.33) | 2.25 × 10⁻¹²³ |

Mental disorders

| | | | |
|---|---|---|---|
| Alcoholic liver damage | 336 | 1.27 (1.20-1.35) | 2.42 × 10⁻¹⁶ |
| Delirium due to conditions | 2338 | 1.23 (1.20-1.26) | 3.81 × 10⁻⁶² |
| classified elsewhere | | | |
| Altered mental status | 4775 | 1.23 (1.21-1.25) | 4.39 × 10⁻¹¹⁷ |
| Alteration of consciousness | 2699 | 1.21 (1.18-1.24) | 1.99 × 10⁻⁵⁶ |
| Aphasia | 843 | 1.17 (1.11-1.23) | 3.13 × 10⁻¹⁰ |

Musculoskeletal

| | | | |
|---|---|---|---|
| Arthropathy associated | 147 | 1.33 (1.19-1.48) | 4.62 × 10⁻⁰⁷ |
| with other disorders | | | |
| classified elsewhere | | | |
| Osteitis deformans | 202 | 1.32 (1.19-1.46) | 6.89 × 10⁻⁰⁸ |
| and osteopathies | | | |
| associated with | | | |
| other disorders | | | |
| classified elsewhere | | | |
| Acute osteomyelitis | 429 | 1.28 (1.21-1.35) | 5.31 × 10⁻¹⁸ |
| Chronic osteomyelitis | 355 | 1.26 (1.19-1.34) | 1.53 × 10⁻¹³ |
| Unspecified osteomyelitis | 662 | 1.26 (1.21-1.31) | 6.71 × 10⁻²⁸ |

Neoplasms

| | | | |
|---|---|---|---|
| Malignant neoplasm | 320 | 1.20 (1.13-1.28) | 2.60 × 10⁻⁰⁸ |
| of liver, primary | | | |
| Bone marrow or | 288 | 1.19 (1.11-1.27) | 5.03 × 10⁻⁰⁷ |
| stem cell transplant | | | |
| Cancer of liver and | 483 | 1.15 (1.10-1.21) | 1.40 × 10⁻⁰⁸ |
| intrahepatic bile duct | | | |
| Myeloproliferative disease | 902 | 1.14 (1.09-1.19) | 2.51 × 10⁻⁰⁸ |
| Cancer of bronchus; lung | 1940 | 1.12 (1.09-1.15) | 1.72 × 10⁻¹⁶ |

Neurological

| | | | |
|---|---|---|---|
| Peripheral autonomic | 343 | 1.21 (1.15-1.29) | 4.73 × 10⁻¹¹ |
| neuropathy | | | |
| Myoclonus | 255 | 1.20 (1.10-1.30) | 1.31 × 10⁻⁰⁵ |
| Disorders of the | 544 | 1.20 (1.14-1.26) | 2.98 × 10⁻¹² |
| autonomic nervous system | | | |
| Coma | 830 | 1.19 (1.12-1.26) | 2.67 × 10⁻⁰⁹ |
| Convulsions | 2105 | 1.17 (1.14-1.20) | 2.47 × 10⁻²⁸ |

Pregnancy complications

| | | | |
|---|---|---|---|
| Hypertension complicating | 531 | 1.18 (1.11-1.25) | 3.01 × 10⁻⁰⁷ |
| pregnancy, childbirth, | | | |
| and the puerperium | | | |
| Malposition and | 424 | 1.15 (1.08-1.23) | 2.15 × 10⁻⁰⁵ |
| malpresentation | | | |
| of fetus or obstruction | | | |

Respiratory

| | | | |
|---|---|---|---|
| Pseudomonal pneumonia | 183 | 1.45 (1.32-1.59) | 1.39 × 10⁻¹⁵ |
| Respiratory arrest | 144 | 1.38 (1.24-1.54) | 1.09 × 10⁻⁰⁸ |
| Obstructive chronic bronchitis | 760 | 1.35 (1.29-1.41) | 6.69 × 10⁻³⁷ |
| Pulmonary congestion | 3879 | 1.35 (1.32-1.38) | 1.94 × 10⁻²¹² |
| and hypostasis | | | |
| Viral pneumonia | 304 | 1.32 (1.23-1.42) | 1.29 × 10⁻¹³ |

Sense organs

| | | | |
|---|---|---|---|
| Other nondiabetic retinopathy | 544 | 1.15 (1.10-1.20) | 2.94 × 10⁻⁰⁹ |
| Blindness and low vision | 1558 | 1.12 (1.08-1.15) | 7.85 × 10⁻¹² |
| Astigmatism | 1941 | 1.11 (1.07-1.16) | 6.66 × 10⁻⁰⁸ |
| Dry eyes | 3087 | 1.08 (1.06-1.10) | 3.35 × 10⁻¹³ |
| Disorders of refraction | 5029 | 1.07 (1.05-1.09) | 1.15 × 10⁻⁰⁹ |
| and accommodation; | | | |
| blindness and low vision | | | |

Symptoms

| | | | |
|---|---|---|---|
| Cardiogenic shock | 582 | 1.55 (1.48-1.62) | 2.96 × 10⁻⁷³ |
| Gangrene | 374 | 1.47 (1.38-1.56) | 1.69 × 10⁻³¹ |
| Shock | 1436 | 1.45 (1.40-1.49) | 1.02 × 10⁻¹¹⁸ |
| Debility unspecified | 475 | 1.21 (1.14-1.29) | 2.01 × 10⁻⁰⁹ |
| Rhabdomyolysis | 366 | 1.19 (1.12-1.27) | 2.31 × 10⁻⁰⁷ |

Injuries and poisonings
*Displayed are significant associations with the top 5 largest effect sizes within each disease category. In cases where there are fewer than 5 significant associations, all significant associations are shown.
^{†}Hazard ratios are presented per 1-point increase in vector component score (see main text)

TABLE 4
Patient report prototype for a 65-year-old female.

| | Probability of | Background |
|---|---|---|
| Phecode | disease (95% CI)* | prevalence (95% CI) |
| Hypertension | 62.1%(60.7-63.4) | 46.8%(45.9-47.6) |
| Diabetes | 22.0%(20.9-23.1) | 15.4%(14.8-16.0) |
| Hyperlipidemia | 51.1%(50.0-52.2) | 42.9%(42.0-43.7) |
| Cardiomyopathy | 16.3%(14.4-18.3) | 5.6%(5.2-6.0) |
| Heart failure with preserved | 2.6%(1.9-3.6) | 0.9%(0.8-1.1) |
| ejection fraction | | |
| Myocardial infarction | 23.5%(20.8-26.3) | 8.0%(7.6-8.5) |
| Paroxysmal ventricular | 6.9%(5.3-9.0) | 2.0%(1.7-2.3) |
| tachycardia | | |
| Mitral and aortic valve | 2.1%(1.5-2.8) | 0.8%(0.6-0.9) |
| stenosis | | |

Various modifications and variations of the described methods of the invention will be apparent to those skilled in the art without departing from the scope and spirit of the invention. Although the invention has been described in connection with specific implementations, it will be understood that it is capable of further modifications and that the invention as claimed should not be unduly limited to such specific implementations. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A system for electrocardiogram-based disease profiling, comprising:
an electrocardiogram analysis module implemented in a non-transitory computer-readable storage medium, the electrocardiogram analysis module configured to perform operations comprising:
deriving disease vectors for a plurality of diseases using electrocardiogram training data from both disease-negative individuals and disease-positive individuals for the plurality of diseases;
generating, via a data preprocessor of the electrocardiogram analysis module, a standardized input from an electrocardiogram recorded from an individual;
encoding, by a deep learning autoencoder of the electrocardiogram analysis module, the standardized input into an embedding, the embedding being a lower-dimensional latent space representation of features extracted from the standardized input; and
generating, by a statistical modeling algorithm of the electrocardiogram analysis module, at least one disease risk score for the individual for the plurality of diseases based on the embedding and the disease vectors.

2. The system of claim 1, wherein the operations further comprise:
reconstructing, via a decoder network of the deep learning autoencoder, the electrocardiogram from the embedding.

3. The system of claim 1, wherein the deep learning autoencoder comprises an encoder network and a decoder network, the encoder network and the decoder network each including multiple convolutional blocks with skip connections.

4. The system of claim 1, wherein deriving the disease vectors for the plurality of diseases comprises, for each disease of the plurality of diseases:
generating, via the deep learning autoencoder, disease-positive embeddings and disease-negative embeddings from the electrocardiogram training data;
plotting the disease-positive embeddings and the disease-negative embeddings in a two-dimensional space;
determining a disease-positive centroid and a disease-negative centroid in the two-dimensional space based on the plotted disease-positive embeddings and the plotted disease-negative embeddings, respectively; and
defining a given disease vector by connecting the disease-negative centroid to the disease-positive centroid in the two-dimensional space.

5. The system of claim 1, wherein generating the standardized input comprises at least one of normalizing, upsampling, or zero-padding the electrocardiogram recorded from the individual.

6. The system of claim 1, wherein generating the at least one disease risk score comprises projecting the embedding onto the disease vectors in the lower-dimensional latent space.

7. The system of claim 1, wherein the electrocardiogram recorded from the individual comprises a 12-lead electrocardiogram.

8. The system of claim 1, wherein the electrocardiogram recorded from the individual comprises a single-lead electrocardiogram.

9. The system of claim 1, wherein the at least one disease risk score indicates a likelihood of the individual having or developing at least one disease of the plurality of diseases within a specified time frame.

10. The system of claim 1, wherein the operations further comprise:
training the deep learning autoencoder using a training sample portion of a model derivation subset of the electrocardiogram training data;
refining the trained deep learning autoencoder using a development sample portion of the model derivation subset of the electrocardiogram training data; and
validating the deep learning autoencoder using both an internal sample portion of the model derivation subset of the electrocardiogram training data and an external test subset of the electrocardiogram training data.

11. The system of claim 10, wherein training the deep learning autoencoder comprises:
generating, by the deep learning autoencoder, a reconstructed electrocardiogram based on an input electrocardiogram from the training sample portion; and
updating weights and biases of the deep learning autoencoder based on a loss between the reconstructed electrocardiogram and the input electrocardiogram from the training sample portion.

12. A method for electrocardiogram-based disease profiling, comprising:
deriving, using a trained deep learning autoencoder, at least one disease vector using electrocardiogram training data from both disease-negative individuals and disease-positive individuals for at least one disease;
generating a standardized input for an electrocardiogram to be processed by the trained deep learning autoencoder;
encoding, via an encoder network of the trained deep learning autoencoder, the standardized input into an embedding, the embedding being a lower-dimensional latent space representation of features extracted from the standardized input;
generating at least one disease risk score based on the embedding and the at least one disease vector, the at least one disease risk score indicating a likelihood of the at least one disease being present; and
reconstructing, via a decoder network of the trained deep learning autoencoder, the electrocardiogram from the embedding.

13. The method of claim 12, wherein generating the at least one disease risk score comprises:
generating a vector component score by projecting the embedding onto a given disease vector of the at least one disease vector in the lower-dimensional latent space; and
generating the at least one disease risk score for the given disease vector based on the vector component score.

14. The method of claim 12, wherein deriving the at least one disease vector comprises:
generating disease-positive embeddings and disease-negative embeddings from the electrocardiogram training data;
plotting the disease-positive embeddings and the disease-negative embeddings in a two-dimensional space;
determining a disease-positive centroid and a disease-negative centroid in the two-dimensional space based on the plotted disease-positive embeddings and the plotted disease-negative embeddings; and
defining the at least one disease vector as a vector connecting the disease-negative centroid to the disease-positive centroid in the two-dimensional space.

15. The method of claim 12, wherein the trained deep learning autoencoder comprises multiple convolutional blocks with skip connections in both of the encoder network and the decoder network.

16. The method of claim 12, wherein generating the standardized input comprises at least one of normalizing, upsampling, or zero-padding the electrocardiogram, and wherein the standardized input comprises a time-series of voltage measurements for each lead of the electrocardiogram that are sampled at a predetermined frequency over a predetermined time period.

17. A method for electrocardiogram-based disease profiling, comprising:
deriving disease vectors for a plurality of diseases using a trained deep learning autoencoder by, for a given disease of the plurality of diseases:
generating, by the trained deep learning autoencoder, disease-positive embeddings from electrocardiograms corresponding to disease-positive individuals for the given disease;
generating, by the trained deep learning autoencoder, disease-negative embeddings from electrocardiograms corresponding to disease-negative individuals for the given disease;
plotting the disease-positive embeddings and the disease-negative embeddings in a two-dimensional space;
determining a disease-positive centroid and a disease-negative centroid in the two-dimensional space based on the plotted disease-positive embeddings and the plotted disease-negative embeddings; and
defining a given disease vector by connecting the disease-negative centroid to the disease-positive centroid in the two-dimensional space; and
generating, using the trained deep learning autoencoder, disease risk scores for the plurality of diseases for an individual based on an electrocardiogram recorded from the individual and the disease vectors.

18. The method of claim 17, wherein generating, using the trained deep learning autoencoder, the disease risk scores for the plurality of diseases for the individual based on the electrocardiogram recorded from the individual and the disease vectors comprises:
generating a standardized input from the electrocardiogram recorded from the individual by at least one of normalizing, upsampling, or zero-padding the electrocardiogram;
encoding, via an encoder network of the trained deep learning autoencoder, the standardized input into an embedding; and
for each disease of the plurality of diseases:
generating a vector component score by projecting the embedding onto the given disease vector; and
generating a corresponding disease risk score for the given disease based on the vector component score.

19. The method of claim 17, wherein the trained deep learning autoencoder comprises an encoder network and a decoder network, and the method further comprises:
reconstructing, via the decoder network of the trained deep learning autoencoder, the electrocardiogram from an embedding generated by the encoder network.

20. The method of claim 17, wherein the trained deep learning autoencoder is trained using unsupervised learning.
